(19) United States
(12) Patent Application Publication
(10) Pub. No.: US 2014/0030234 A1
(43) Pub. Date: Jan. 30, 2014

(54) METHODS AND COMPOSITIONS FOR MODULATING ISLET BETA CELL DEVELOPMENT

(71) Applicant: The Board of Trustees of the Leland Stanford Junior University, Palo Alto, CA (US)

(72) Inventors: Seung K. Kim, Stanford, CA (US); William Goodyer, Stanford, CA (US); Gerald R. Crabtree, Woodside, CA (US); Pei Wang, Palo Alto, CA (US)

(21) Appl. No.: 13/940,135

(22) Filed: Jul. 11, 2013

Related U.S. Application Data
(60) Provisional application No. 61/670,813, filed on Jul. 12, 2012.

(51) Int. Cl. C12N 5/071; A61K 35/39

(52) U.S. Cl. CPC C12N 5/0676; A61K 35/39
USPC 424/93.7; 435/34; 435/377; 435/6.12; 435/7.92; 435/7.21; 514/291; 514/6.9

(57) ABSTRACT

Methods and compositions are provided for modulating pancreatic islet β-cell development. Aspects of the methods include promoting β-cell development by providing agents that promote calcineurin/N FAT signaling, and inhibiting β-cell development by providing agents that inhibit calcineurin/NFAT signaling. These methods find a number of uses, including, for example, in the treatment of diabetes and human islet diseases. In addition, reagents, devices and kits thereof that find use in practicing the subject methods are provided.

### CROSS REFERENCE TO RELATED APPLICATIONS

Pursuant to 35 U.S.C. §119 (e), this application claims priority to the filing date of the U.S. Provisional Patent Application Ser. No. 61/670,813 filed Jul. 12, 2012, the full disclosure of which is herein incorporated by reference.

### GOVERNMENT RIGHTS

This invention was made with Government support under contract DK075919 awarded by the National Institutes of Health. The Government has certain rights in this invention.

### FIELD OF THE INVENTION

This invention pertains to modulating pancreatic islet β cell development.

### BACKGROUND OF THE INVENTION

Defects in β cell function and number underlie many human diseases. Emerging strategies to achieve replacement or regeneration of pancreatic β cells in the case of loss of β cell mass or function, or to attenuate growth and development in the case of β cell hypertrophy or hyperactivity, rely on tools to modulate β cell development and growth. The present invention addresses these issues.

### SUMMARY OF THE INVENTION

Methods and compositions are provided for modulating pancreatic islet β-cell development. Aspects of the methods include promoting β-cell development by providing agents that promote calcineurin/N FAT signaling, and inhibiting β-cell development by providing agents that inhibit calcineurin/NFAT signaling. These methods find a number of uses, including, for example, in the treatment of diabetes and human islet diseases. In addition, reagents, devices and kits thereof that find use in practicing the subject methods are provided.

In some aspects of the invention, methods are provided for modulating β cell development. In these methods, progenitor cells are contacted with an effective amount of an agent that modulates calcineurin/NFAT signaling. In some embodiments, the agent promotes calcineurin/N FAT signaling, and β cell development is promoted. In other embodiments, the agent antagonizes calcineurin/NFAT signaling, and β cell development is inhibited. In some embodiments, the progenitor cell is an embryonic stem (ES) cell, an induced pluripotent stem cell (iPSC), an endocrine progenitor cell (EPC), or a pancreatic duct cell. In some embodiments, the method is performed in vitro. In other embodiments, the method is performed in vivo. In some embodiments, the progenitor cell is from a neonate or a juvenile, or is derived from a cell from a neonate or a jeuvenile. In other embodiments, the progenitor cell is from an adult, or is derived from a cell from an adult. In certain embodiments, the method further comprises assaying for mature β cells or for the development of mature β cells, for example by quantifying the number of mature β cells before and after the contacting, or by quantifying the amount of a factor that is produced by mature β cells, e.g. insulin, C-peptide, amylin, granin, IA2, or the like.

In some aspects of the invention, methods of modulating β cell development are applied to producing an enriched composition of mature β cells in vitro, the method comprising contacting a progenitor cell in vitro with an effective amount of an agent that promotes calcineurin/N FAT signaling. In some embodiments, the method further comprises contacting the mature β cell with an agent that promotes mature β cell expansion. In some embodiments, the method further comprises enriching the composition for mature β cells by affinity separation. In some aspects of the invention, an enriched composition of mature β cells is provided.

In some aspects of the invention, an enriched composition of mature β cells prepared by the subject methods is used in methods of treating an individual in need of functional β cells, the methods comprising transplanting the enriched population of mature β cells into the individual. In some embodiments, the progenitor cell is from the individual. In some embodiments, the progenitor cell is derived from a cell from the individual. In some embodiments, the individual has diabetes.

In some aspects of the invention, methods of modulating β cell development are applied in vivo to modulate the number, or “mass”, of mature β cells in an individual. In some embodiments, method comprises contacting pancreatic tissue in vivo with an effective amount of an agent that modulates calcineurin/NFAT signaling. In some embodiments, the agent promotes calcineurin/N FAT signaling, and β cell development is promoted. In some such embodiments, the individual has diabetes. In other embodiments, the agent inhibits calcineurin/NFAT signaling, and β cell development is suppressed. In some such embodiments, the individual has insulinoma, hypoglycemia, or an acquired state of β cell overgrowth. In certain embodiments, the method further comprises assaying for mature β cells or for the development of mature β cells, for example by quantifying the number of mature β cells before and after the contacting or by quantifying the amount of a factor that is produced by mature β cells, e.g. insulin, C-peptide, amylin, granin, IA2, or the like.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. nCnb1KO Mice Develop Severe Postnatal Diabetes, Hypoinsulinemia, and Early Onset Lethality. (A and B) Representative insulin immunostaining and quantification of total b cell area/Pancreatic area (in percentage) in postnatal day 1 (P1) control (black bar) and nCnb1KO (gray bar) pancreas (n=3 per genotype). (C) Blood glucose levels of postnatal nCnb1KO mice (gray lines) and littermate controls (black lines) during ad libitum feeding (n=4 per genotype minimum per time point). (D) Percent survival of aging mice (n=31, controls, black lines; n=14, nCnb1KO, gray lines). (E) Glucose tolerance test performed on P19, normoglycemic mice (n=5, controls, black; n=4, nCnb1KO, gray). Inset, area under the curve calculated for indicated genotypes. (F and G) Serum insulin (F) and serum glucagon (G) levels from fasted P26 mice. (H) a cell mass in P26 mice. All data are from both female and male mice and represented as means±SEM. *p<0.05, **p<0.025, ***p<0.002. §, not significant (n.s.). See also FIG. 8.

FIG. 2. Decreased Insulin Production and Secretion in nCnb1KO Islets. (A) Whole islet insulin content by insulin EIA in size-matched control (black bars) and nCnb1KO (gray bars) islets assessed on postnatal day 20 (P20). (B) Glucose-stimulated (left) and arginine-stimulated (right) insulin secretion in static culture assays of islets from P20, normoglycemic nCnb1KO and control mice. (C) Quantitative real-time PCR (qRT-PCR) of b cell factors involved in insulin production and secretion, including Insulin 2 (Ins2), pancreatic and duodenal homeobox 1 (Pdx1), glucose transporter 2 (Glut2), and glucokinase (Gck) in P20 nCnb1KO islets as compared to size-matched islets from littermate controls (n=4 per genotype). All data presented as means±SEM. *p<0.05, **p<0.025, ***p<0.002. §, not significant (n.s.). (D) Immunohistochemical detection of b cell factors Insulin and Glut2 in P20 nCnb1KO and control pancreatic islets. Scale bar=10 mM. See also FIG. 9.

FIG. 3. Dense Core Granule Biogenesis and Maturation in Mouse b Cells Requires Cn/NFAT Signaling (A) Transmission electron micrographs of postnatal day 20 (P20) control and nCnb1KO b cells. (B) Representative pictures of the four insulin granule types: (1) mature, (2) immature, (3) crystal-containing, and (4) empty. (C and D) Quantification and morphometric analysis of dense core granules (DCGs) from WT (black bars) and nCnb1KO (gray bars) b cells showing (C) number of granules per unit area and (D) abundance of the different granule subtypes (as a percentage of the total number of granules). (E) qRT-PCR of DCG components in P20 nCnb1KO islets as compared to size-matched islets from littermate controls (n=4 per genotype). Dashed line represents control levels normalized to 1.0. (F) qRT-PCR of β cell factors and DCG components in P20 islets from wild-type (WT), C57BL/6 male mice treated with FK506 (10 mM) or vehicle (EtOH) for 72 hr (n=5). (G-J) Immunohistochemical detection of DCG components ChgA (G), ChgB (H), IAPP (I), and IA2 (J) in P26 nCnb1KO and control islets. Scale bar=10 mM. (K) Chromatin immunoprecipitation (ChIP) of NFATc1 at indicated loci in islets isolated and fixed from P20 WT, C57BL/6 mice. Islets were treated for 24 hr with either vehicle (EtOH) or FK506 (10 mM) (n=4 per condition). ChIP data are presented as fold change of signal relative to IgG background with comparisons to leftmost data bar (black). (L) Relative mRNA levels of indicated genes after in vitro transfection of MIN6 cells with human NFATc1 expression construct (hNFATc1) or empty expression vector (Vector) and treated with either vehicle DMSO or a combination of lonomycin and PMA (I/P). All data presented as means±SEM. *p<0.05, **p<0.025, ***p<0.002. x, not significant (n.s.)

FIG. 4. Cn/NFAT Signaling Regulates Expression of DCG Components in Human Islets. (A) Relative quantification of mRNAs encoding indicated DCG components in isolated human islets (n=3) treated with FK506 (10 μM) or vehicle (EtOH) for 72 hr. Dashed line represents vehicle-treated control levels normalized to 1.0. (B) ChIP of NFATc1 on isolated human islets (sample #1: 5 years old, sample #2: 13 years old). Each human sample was divided and treated for 24 hr with either vehicle (EtOH) or FK506 (10 μM). (C) Additional adjacent NFAT consensus sites (“Site #2”) within the indicated gene promoter regions did not bind NFATc1 (see also Table 2). ChIP data are presented as fold change of NFATc1 signal (white bar) or NFATc1+FK506 (gray bar) relative to IgG (black bar) control signal. All data presented as means±SEM. *p<0.05, **p<0.025, ***p<0.002. †p<0.15. §, not significant (n.s.).

FIG. 5. Mouse Neonatal β Cell Proliferation and Mass Regulated by Cn/NFAT Signaling (A) Representative insulin stains of Control and nCnb1KO pancreatic tissue at postnatal day 26 (P26). (B) Quantification of β cell mass by morphometry in control (black bar) and nCnb1KO (gray bar) mice. (C) Quantification of β cell proliferation by scoring the percentage of Ki67+β cells in control (black bar) and nCnb1KO (gray bar) pancreatic islets. (D) Quantification of mRNAs encoding indicated cell cycle regulators in P20 nCnb1KO islets and size-matched control islets (n=4 per genotype). Dashed line represents control levels normalized to 1.0. (E) mRNA quantification of CcnA2, CcnD2, and FoxM1 in P20 islets from WT C57BL/6J male mice treated with FK506 (10 mM) or vehicle (EtOH) for 72 hr (n=5). Dashed line represents control levels normalized to 1.0. (F and G) Immunohistochemical detection of Cyclin D2 (F), gray, or red in merge), and FoxM1 (G), gray, or red in merge) in P26 nCnb1KO and control pancreatic islets. Insulin (Ins) in green. Scale bar=10 mM. (H and I) qRT-PCR time course of cell cycle regulators in FACS-isolated b cells from MIP-GFP mice at indicated ages (n=4, 2, 3, 2, 2 at each time point, respectively). (J) ChIP of NFATc1 on indicated loci from islets isolated and fixed from P20 wild-type, C57BL/6 mice. Islets were treated for 24 hr with either vehicle (EtOH) or FK506 (10 mM) (n=4 per condition). ChIP data are presented as fold change of NFATc1 (white bar) or NFATc1+FK506 (gray bar) signal relative to IgG (black bar) background signal. (K) Quantification of mRNA levels of indicated genes after in vitro transfection of MIN6 cells with human NFATc1 expression construct (hNFATc1) or empty expression vector (Vector) treated with either lonomycin and PMA (I/P) or vehicle DMSO. All data presented as means±SEM. *p<0.05, **p<0.025, ***p<0.002. §, not significant (n.s.), with comparisons to leftmost data bar (black), unless otherwise noted.

FIG. 6. CCNA2, CCND2, and FOXM1 mRNA Levels Peak during the Neonatal Period in Human Islets, and Cn/NFAT Regulates Their Expression. (A-D) qRT-PCR time course of CCNA2, CCND2, FOXM1, and CDK2 mRNA transcript levels in isolated islets from humans of increasing age (n=2, except time point “39-56 y”, where n=4). (E) ChIP of NFATc1 at indicated loci from isolated human islets (sample #1: 5 years old, sample #2: 13 years old) treated for 24 hr with either vehicle (EtOH) or FK506 (10 mM). Note: Sample #1 was only treated with vehicle because of limited islet yield from donor sample. Additional adjacent NFAT consensus sites (“Site #2”) within the gene promoter region did not bind NFATc1 (see also Table 2). ChIP data are presented as fold change of NFATc1 signal (white bar) or NFATc1+FK506 (gray bar) relative to IgG (black bar) control signal. (F) mRNA quantification of indicated genes in isolated human islets (n=3) treated with FK506 (10 mM) or vehicle (EtOH) for 72 hr. Dashed line represents vehicle treated control levels normalized to 1.0. (G and H) Quantification of BrdU+ insulin+ cells as a percentage of all insulin+ cells in islets isolated from a 4-year-old human donor pancreas. Islets were divided and exposed to vehicle (DMSO) or FK506 (10 mM) (see Experimental Procedures). (H) Representative immunofluorescence staining of insulin+BrdU+ double positive cells (arrowheads) from 4-year-old donor islets. Insulin (green) and BrdU (red). All data presented as means±SEM. *p<0.05, **p<0.025, ***p<0.002. §, not significant (n.s.).

FIG. 7. Glucokinase Activator Induces Transcription of NFATc1 and Its Targets in a Calcineurin-Dependent Manner (A-D) Islets isolated from postnatal (P10) control C57Bl/6 or nCnb1KO mice treated with either vehicle, glucokinase activator (GKA) R0-28-1675 (10 mM) or GKA+FK506 (10 mM each) for 72 hr (n=3 minimum per condition). qRT-PCR of (A) NFATc1, (B) Insulin 2, (C) indicated DCG components, and (D) indicated cell cycle regulators. (E) Schematic summarizing a role for Cn/NFAT signaling in postnatal b cell (1) maturation and (2) proliferation via the direct transcriptional regulation of key b cell genes. Ins2 (insulin 2), Pdx1 (pancreatic duodenal homeobox 1), Glut2 (glucose transporter type 2), Gck (glucokinase), ChgA/B (chromogranins A and B), IAPP (islet amyloid polypeptide), IA2 (ICA512), CcnA2 (cyclinA2), CcnD2 (cyclinD2), and FoxM1 (forkhead homeobox factor M1). All data are from male mice and are represented as means±SEM. *p<0.05, **p<0.025, ***p<0.002. x, not significant (n.s.). See also FIG. 10.

FIG. 8. Additional physiologic and cellular analyses of nCnb1KO mice. A. Mating scheme for generating Ngn3-Cre; Cnb1Δ/f (nCnb1KO) mice. B. Representative immunofluorescent stains demonstrating normal islet cell composition in postnatal day 1 (P1) nCnb1KO islets as compared to littermate controls. DAPI in blue; Ins (Insulin) in green; Gluc (Glucagon), Som (Somatostatin), PP (Pancreatic polypeptide), and Ghr (Ghrelin) in red. C. Quantitative real-time RT-PCR (QPCR) of Calcineurin b1 (Cnb1) and hypoxiainducible factor 1 (Hif1a) transcript levels from islets isolated from postnatal day 20 control and nCnb1KO islets. D. Immunohistology of NFATc1 within P20 control and KO pancreatic islets. DAPI in blue and NFATc1 in red. Insets are of single β-cells illustrating nuclear localization of NFATc1 in controls and cytoplasmic localization in nCnb1KOs. E. Body mass (in g) of littermate mice (n=8 min. per genotype). F. Insulin tolerance test (ITT) in postnatal day 20 mice (mixed sex; Control, n=6; nCnb1KO, n=4) injected intraperitoneally with insulin (1 U/kg). Blood glucose levels were measured at times 0, 15, 30 and 45 min. G. Quantification of the percentage of β-cells positive for activated caspase 3 staining in either control (black bar) or nCnb1KO (grey bar). Scale bar=10 μM. All data presented as means±s.e.m. **, P<0.025. ***, P<0.002. §, not significant (n.s.)

FIG. 9. Other pancreatic endocrine cell types are grossly unaffected in diabetic nCnb1KO mice. Representative immunofluorescent stains of pancreatic islets from diabetic, postnatal day 26 (P26) nCnb1KO mice and littermate controls. DAPI in blue; Ins (Insulin—β-cells) in green; Som (Somatostatin—Delta cells), PP (Pancreatic polypeptide—PP cells), and Ghr (Ghrelin—Epsilon cells) in red. Scale bar=10 μM.

FIG. 10. pCnb1KO mice phenocopy the severe postnatal, diabetic phenotype of nCnb1KO mice. A. Quantification of total β-cell Area/Pancreatic Area (in percentage) in postnatal day 1 (P1) control (black bar) and Pdx1-Cre; Cnb1Δ/f (pCnb1KO) (grey bar) pancreas (n=3 per genotype). B. Random fed blood glucose levels of postnatal nCnb1KO mice (grey bars) and littermate controls (black bars) fed ad libitum (n=3 per genotype min. per timepoint). All data are from both female and male mice and are represented as means±s.e.m. **, P=0.006. §, not significant (n.s.).

FIG. 11. NFATc1 binding of mouse Ins2 and Gck promoter regions in vivo by ChIP. Chromatin immunoprecipitation (ChIP) of NFATc1 on islets isolated and fixed from postnatal day 20 C57BL/6 mice. Islets were treated for 24 hrs with either vehicle (EtOH) or FK506 (10 μM) (n=4 per condition). Putative NFAT consensus sites were assessed in the upstream (within 2 kb from start site) promoter regions of Insulin 2 (Ins2) and Glucokinase (Gck). ChIP data is presented as Fold Change of signal relative to IgG background signal. All data presented as means±s.e.m. *, P<0.05. **, P<0.025. §, not significant (n.s.).

FIG. 12. Decreased Pdx1 and CcnA2 protein expression in nCnb1ko β-cells. Representative immunohistology of insulin or glucagon (green in merge) and Pdx1 (A) or CyclinA2 (B) (red in merge) in pancreatic islets from postnatal day 26 control and nCnb1KO mice. Scale bar is 10 μM.

FIG. 13. NFATc1, NFATc2 and NFATc4 mRNA transcript levels are enriched in postnatal islets. A. Relative mRNA transcript levels of Cn/NFAT signaling components by QPCR in islets isolated from early postnatal day 10 (P10) (grey bars) vs. mature (P28) (white bars) CD1 mice. B. Relative mRNA levels of NFATc1 in FAC-sorted β-cells from MIP-GFP mice of indicated ages (in days) on x-axis. Calcineurin A (CnA), Calcineurin b1 (Cnb1). All data are from male mice and are represented as means±s.e.m. *, P<0.05. **, P<0.025. ***, P<0.002. §, not significant.

FIG. 14. Treatment of ES cells with glucokinase activator (GKA) promotes the expression of genes expressed by mature β cells. Two independent experiments were performed. A. Experiment 1. B. Experiment 2. PDX1: pancreatic duodenal homeobox 1; Ins: insulin; CHRA: chromogranin A.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and compositions are provided for modulating pancreatic islet β-cell development. Aspects of the methods include promoting β-cell development by providing agents that promote calcineurin/N FAT signaling, and inhibiting β-cell development by providing agents that inhibit calcineurin/NFAT signaling. These methods find a number of uses, including, for example, in the treatment of diabetes and human islet diseases. In addition, reagents, devices and kits thereof that find use in practicing the subject methods are provided. These and other objects, advantages, and features of the invention will become apparent to those persons skilled in the art upon reading the details of the compositions and methods as more fully described below.

Methods and compositions are provided for modulating pancreatic islet β cell development. By pancreatic islet β cell development it is meant the development, i.e. differentiation, of a progenitor cell into a mature pancreatic islet β cell.

The pancreas serves two major functions: (i) the production of digestive enzymes, which are secreted by exocrine acinar cells and routed to the intestine by a branched ductal network; and (ii) the regulation of blood sugar, which is achieved by endocrine cells of the islets of Langerhans. Several separate endocrine cell types comprise the islet. Pancreatic β cells, also referred to as β-cells or “beta cells”, are the most prominent (50-80% of the total, depending on species); they produce a number of polypeptides including insulin, a hormone that controls the level of glucose in the blood; C-peptide, a byproduct of insulin production, which helps to prevent neuropathy and other symptoms of diabetes related to vascular deterioration; and amylin, also known as islet amyloid polypeptide (IAP, or IAPP), which functions as part of the endocrine pancreas and contributes to glycemic control. Glucagon-producing α-cells are the next most-common cell type. The remaining islet cells, each comprising a small minority of the total, include δ-cells, which produce somatostatin; PP cells, which produce pancreatic polypeptide; and ε-cells, which produce ghrelin. Without wishing to be bound by theory, it is believed that both exocrine and endocrine cells of the pancreas derive from a common pancreatic progenitor, a Pdx1 and Ptf1a-expressing endodermal cell that becomes specified from foregut endoderm by the expression of genes including Hlxb9, Ptf1a, Tcf2, Hnf6 and Hes1. This progenitor is capable of proliferating to produce more pancreatic progenitors and of differentiating into acini cells, duct cells, and islet cells. In the course of differentiating into islet cells, the pancreatic progenitor begins to express Ngn3; islet subtypes are then believed to be specified from this common endocrine progenitor by the expression of combinations of different transcription factors, including, for β cell specification, Pax6, Pax4, Nx2.2, Nkx6.1, and Hlxb9. For more details, see, e.g., Murtaugh, L (2007) Pancreas and beta-cell development: from the actual to the possible. Development 134, 427-438, the disclosure of which is incorporated herein by reference.

As discussed above, mature β cells store and release a number of factors into the body, including, for example, insulin, C-peptide, islet amyloid polypeptide (amylin), granins, e.g., chromogranin A (ChgA) and chromogranin B (ChgB), and transmembrane proteins such as IA2 (also called ICA152). As such, mature β cells may be readily distinguished from other cells of the pancreas by the presence of dense core secretory granules (DCGs) which contain proteins such as insulin and islet amyloid polypeptide, granins, e.g., chromogranin A (ChgA) and chromogranin B (ChgB), and transmembrane proteins such as IA2 (also called ICA152). In addition, mature β cells may readily be identified based on their expression of genes crucial for the production and secretion of insulin, including insulin 2, pancreatic duodenal homeobox 1 (Pdx1), type 2 glucose transporter (glut2), and glucokinase (Gck). Mature β cells in the expansion phase of islet development also express certain known cell cycle regulators, including Ccnd1, Ccnd2, Cdk4 and FoxM1, which may serve as markers for these cells.

In aspects of the subject methods, β cell development is modulated by modulating the activity of the calcineurin pathway. Calcineurin (CN), also known as “protein phosphatase 3” and “calcium-dependent serine-threonine phosphatase,” is a serine/threonine protein phosphatase. It is a heterodimer of a 61-kD calmodulin-binding catalytic subunit (calcineurin A) and a 19-kD Ca2+-binding regulatory subunit (calcineurin B). There are three isoforms of the calcineurin A catalytic subunit, each encoded by a separate gene (PPP3CA (Genbank Accession No. NM_{—}000944), PPP3CB (Genbank Accession No. NM_{—}021132), and PPP3CC (Genbank Accession No. NM_{—}005605) and two isoforms of the calcineurin B regulatory subunit, each also encoded by separate genes (PPP3R1 (Genbank Accession No. NM_{—}000945), and PPP3R2 (Genbank Accession No. NM_{—}147180)). In the most studied pathway of calcineurin regulation of cell activity, an intracellular increase of calcium ions in a cell activates the protein calmodulin (CaM), which in turn activates calcineurin. Calcineurin in turn activates nuclear factor of activated T cell, cytoplasmic (NFATc), a transcription factor, by dephosphorylating it. The activated NFATc then translocates into the nucleus, where it upregulates the expression of target genes (Nature. 1992 Jun. 25; 357(6380):695-7). There are 4 known cytoplasmic NFAT proteins: NFATc1 (also called NFATc), NFATc2 (also called NFATp or NFAT1), NFATc3 (also called NFATx or (4)), and NFATc4 (also called NFAT3).

In some aspects of the subject methods, β-cell development is promoted, and insulin secretion is increased. In such methods, progenitor cells are contacted with an agent that promotes, i.e. enhances or augments, the activity of the calcineurin/NFATc1 signaling pathway. In other words, the cell is contacted with a calcineurin/NFATc1 agonist. Agents that promote calcineurin/NFATc1 signaling include agents that activate or increase the activity of calcineurin, e.g. by activating calcineurin directly or by promoting the activity of proteins upstream of calcineurin. Non-limiting examples of such agents would include proteins or small molecules that raise intracellular levels of calcium ions, e.g. the ionophore ionomycin or calcimycin (A23187); agents that promote glucokinase activity, e.g. glucokinase polypeptide and activators thereof, e.g. small molecule glucokinase activators such as R0-28-1675, RO4597014, MK-0941, LY2599506, LY2121260, YH-GKA, AMG 151, and AZD1656; and the peptide glucagon-like peptide 1 (GLP1) or variants thereof. Agents that promote calcineurin/NFATc1 signaling also include agents that act on proteins downstream of calcineurin, e.g. agents that activate or increase the activity of NFAT, e.g. phorbol-12 myristate-13 acetate (PMA), NFAT cDNA, or an NFAT polypeptide.

In other aspects of the subject methods, β cell development is inhibited by contacting the progenitor cells with an agent that antagonizes, i.e. suppresses, inhibits, attenuates, or negatively regulates, calcineurin/NFATc1 signaling. Agents that antagonize calcineurin/NFATc1 signaling include agents that suppress the activity of calcineurin, for example by inhibiting calcineurin directly, e.g. tacrolimus (FK506) or pimecrolimus, or by inhibiting the activity of proteins upstream of calcineurin, e.g. cyclosporine A. Agents that antagonize calcineurin/NFATc1 signaling also include agents that inhibit the activation of or activity of proteins downstream of calcineurin, e.g. NFAT peptide inhibitor 11R-VIVIT (MAGPHPVIVITGPHEE), or the small molecule inhibitors INCA-1, INCA-2, and INCA-6 (Roehrl et al. (2004) Selective inhibition of calcineurin-NFAT signaling by blocking protein-protein interaction with small organic molecules. PNAS May 18, 2004 vol. 101 no. 20 7554-7559).

Any agent that modulates, i.e. promotes or antagonizes, the activity of the calcineurin/NFAT signaling pathway may be employed to modulate β cell maturation in the subject methods. For example, small molecule compounds may be used. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992). Small molecule compounds can be provided directly to the medium in which the cells are being cultured, for example as a solution in DMSO or other solvent.

Agents that modulate calcineurin/N FAT signaling that would be suitable for use in the subject methods also include nucleic acids, for example, nucleic acids that encode siRNA, shRNA or antisense molecules, e.g. that are specific for genes in the calcineurin/NFAT pathway, e.g. in instances in which calcineurin/NFAT signaling is to be suppressed; or nucleic acids that encode polypeptides, e.g. calcineurin/N FAT polypeptides, e.g. in instances in which calcineurin/NFAT signaling is to be promoted. Many vectors useful for transferring nucleic acids into target cells are available. The vector may be maintained episomally, e.g. as plasmid, minicircle DNA, virus-derived vector such as cytomegalovirus, adenovirus, etc., or it may be integrated into the target cell genome, through homologous recombination or random integration, e.g. retrovirus derived vectors such as MMLV, HIV-1, ALV, etc. The nucleic acid agent may be provided directly to the progenitor cells. In other words, the progenitor cells are contacted with vectors comprising the nucleic acid of interest such that the vectors are taken up by the cells. Methods for contacting cells with nucleic acid vectors, such as electroporation, calcium chloride transfection, and lipofection, are well known in the art. Alternatively, the nucleic acid agent may be provided to progenitor cells via a virus. In other words, the progenitor cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject progenitor cells are targeted by the packaged viral particles. Methods of introducing the retroviral vectors comprising the nucleic acid calcineurin modulator into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by 5 fold or more, by 10 fold or more, by at least about 100 fold or more, more usually by at least about 1000 fold. In addition, vectors used for providing nucleic acid to the subject cells may include genes that must later be removed, e.g. using a recombinase system such as Cre/Lox, or the cells that express them destroyed, e.g. by including genes that allow selective toxicity such as herpesvirus TK, bcl-xs, etc

Agents suitable for modulating calcineurin/NFAT signaling in the present invention also include polypeptides and peptides. Such polypeptides and peptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the polypeptide or peptide agent is to modulate calcineurin signaling intracellularly, the polypeptide may comprise the polypeptide sequences of interest fused to a polypeptide permeant domain. A number of permeant domains are known in the art and may be used in the polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the polypeptide or peptide agent is to modulate calcineurin signaling by modulating the activity of a transmembrane protein, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

Stable plasma proteins are proteins which typically exhibit in their native environment an extended half-life in the circulation, i.e. greater than about 20 hours. Examples of suitable stable plasma proteins are immunoglobulins, albumin, lipoproteins, apolipoproteins and transferrin. The polypeptide agent typically is fused to the plasma protein, e.g. IgG at the N-terminus of the plasma protein or fragment thereof which is capable of conferring an extended half-life upon the polypeptide. Increases of greater than about 100% on the plasma half-life of the polypeptide are satisfactory. Ordinarily, the polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods. The site at which the fusion is made may be selected in order to optimize the biological activity, secretion or binding characteristics of the polypeptide. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

The polypeptide agent for use in the subject methods may be produced from eukaryotic produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Another example of polypeptide agents suitable for modulating calcineurin signaling are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The modulator of calcineurin/NFAT signaling activity (the “calcineurin/NFAT signaling modulator”, or “calcineurin/N FAT modulator”) is provided to cells in an effective amount, i.e. an amount that is effective to modulate calcineurin signaling and hence, β cell development. Biochemically speaking, an effective amount or effective dose of a calcineurin/N FAT signaling modulator is an amount of modulator necessary to alter calcineurin/NFAT signaling in a cell by 30% or more, 40% or more, 50% or more, 60% or more, 70% or more, 80% or more, 90% or more, 100% or more, 200% or more, or 500% or more. In other words, the activity of the calcineurin/N FAT signaling pathway in a cell contacted with an effective amount or effective dose of a calcineurin/NFAT signaling antagonist will be about 70% or less, about 60% or less, about 50% or less, about 40% or less, about 30% or less, about 20% or less, about 10% or less, about 5% or less, or will be about 0%, i.e. negligible, the activity observed in a cell that has not been contacted with an effective amount/dose of a calcineurin/N FAT signaling antagonist, while the activity of the calcineurin/N FAT signaling pathway in a cell contacted with an effective amount or effective dose of a calcineurin/NFAT signaling agonst will be about 30% or more, 40% or more, 50% or more, 60% or more, 70% or more, 80% or more, 90% or more, 100% or more, 200% or more, or 500% or more. Put another way, calcineurin/NFAT signaling will be altered about 0.5-fold or more, 1-fold or more, 2-fold or more, 5-fold or more, 8-fold or more, or 10-fold or more.

The extent to which a cell's activity is modulated by a calcineurin/NFAT signaling modulator can be readily determined by a number of ways known to one of ordinary skill in the art of molecular biology. For example, changes in the level of expression of genes known to be upregulated in mature β cells, e.g. Ins1, Ins2, ChgA, ChgB, IAPP, IA2, Ccnd2, FoxM1, and CcnA2 made be measured by RT-PCR, Northern Blot, RNAse protection, Western blot, ELISA, and the like. The amount of NFAT associated with the cis-regulatory elements of β cell genes upregulated by NFAT, e.g. ChgA, ChgB, and IA2, may be assessed by, e.g. Chromatin IP (ChIP). The number of dense core granules (DCGs) may be measured, where an increase in the number of DCGs is indicative of β cell maturation. The amount of insulin, C-peptide, or IAPP produced by the cells may be quantified, e.g. by Western blot or ELISA, e.g. before and after contacting with modulator and compared. In this way, the modulatory effect of the agent may be confirmed.

In a clinical sense, an effective dose of a calcineurin/NFAT modulator is the dose that, when administered for a suitable period of time, usually at least about one week, and maybe about two weeks, or more, up to a period of about 4 weeks, 8 weeks, or longer will evidence an alteration the symptoms associated with β cell dysfunction or disorder. For example, an effective dose of a calcineurin/NFAT agonist is the dose that when administered for a suitable period of time, usually at least about one week, and may be about two weeks, or more, up to a period of about 4 weeks, 8 weeks, or longer will promote the maturation of β cells and production of insulin in a patient suffering from diabetes. As another example, an effective dose of a calcineurin/N FAT antagonist is the dose that when administered for a suitable period of time, usually at least about one week, and may be about two weeks, or more, up to a period of about 4 weeks, 8 weeks, or longer will slow, halt or reverse the maturation of β cells and production of insulin in a patient suffering from insulinoma, mixed endocrine tumor, or acquired states of β cell overgrowth. It will be understood by those of skill in the art that an initial dose may be administered for such periods of time, followed by maintenance doses, which, in some cases, will be at a reduced dosage.

Calculating the effective amount or effective dose of calcineurin/NFAT modulator to be administered is within the skill of one of ordinary skill in the art, and will be routine to those persons skilled in the art. Needless to say, the final amount to be administered will be dependent upon a variety of factors, include the route of administration, the nature of the disorder or condition that is to be treated, and factors that will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally or topically administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

The subject methods may be used to promote or suppress β cell development in a variety of progenitor cells, for example, pluripotent stem cells, or certain types of somatic cells. By “pluripotent stem cell” or “pluripotent cell” it is meant a cell that a) can self-renew and b) can differentiate into all types of cells in an organism. Pluripotent cells are capable of forming teratomas and of contributing to ectoderm, mesoderm, or endoderm tissues in a living organism. By “somatic cell” it is meant any cell in an organism that can self-renew, but that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both β cells and pancreatic or endocrine progenitor cells, the latter of which may be able to naturally give rise to all or some cell types of the pancreas but cannot give rise to cells of the ectoderm, mesoderm or endoderm lineages.

Examples of pluripotent stem cells include embryonic stem (ES) cells and induced pluripotent stem (iPS) cells. By “embryonic stem cell” or “ES cell” it is meant a pluripotent stem cell that is derived from the inner cell mass of the blastula of a developing organism. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. No. 7,029,913, U.S. Pat. No. 5,843,780, and U.S. Pat. No. 6,200,806, the disclosures of which are incorporated herein by reference. By “induced pluripotent stem cell” or “iPS cell” it is meant a pluripotent stem cell that is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPS cells may be found in, for example, Application Nos. US20090047263, US20090068742, US20090191159, US20090227032, US20090246875, and US20090304646, the disclosures of which are incorporated herein by reference. In some instances, the pluripotent stem cell is from an individual to be treated by the subject methods. In some instances, the pluripotent stem cell is derived from a cell from the individual to be treated by the subject methods.

Another example of a progenitor cell that finds use in the subject methods is a pancreatic progenitor cell. A pancreatic progenitor cell, as used herein, encompasses any somatic cell that has the potential to give rise to all cells of the pancreas. Pancreatic progenitor cells have the potential to become acini cells, duct cells, and islet cells, and are readily identifiable by their expression of Sox9 and Pdx1.

Another example of a progenitor cell that finds use in the subject methods is an endocrine progenitor cell (EPC). An endocrine progenitor cell, as used herein, encompasses any somatic cell that has the potential to give rise to any endocrine cells of the pancreas, i.e., β cells (insulin+), α cells (glucagon+), PP-cell, δ cell (somatostatin+), ε-cell (ghrelin+). Endocrine progenitor cells are duct-like epithelial cells centrally located in the pancreas, and readily identifiable by their expression of Ngn3 and their lack of expression of hormones such as glucagon, insulin, pancreatic polypeptide, grehlin, somatostatin, i.e. Ngn3-positive, glucagon-negative, insulin-negative, pancreatic polypeptide-negative, grehlin-negative, and somatostatin-negative cells.

Another example of a progenitor cell that may be used in the subject methods is a pancreatic duct cell. Pancreatic duct cells, also known as pancreatic ductal epithelial cells (PDCs), are somatic cells that form the epithelial lining of the branched tubes (ducts) that deliver enzymes produced by pancreatic acinar cells into the duodenum. In addition, these cells secrete bicarbonate that neutralizes stomach acidity. Pancreatic duct cells express one or more of CK19, CA19-9, and PDX1, and may be isolated from dispersed islet-depleted human pancreatic tissue using CA19-9 antibody. Pancreatic duct cells may be cultured by methods known in the art; see, e.g. Schreiber et al. (2004) Successful growth and characterization of mouse pancreatic ductal cells: functional properties of the Ki-RAS(G12V) oncogene. Gastroenterology 127:250-260, the disclosure of which is incorporated herein by reference.

In some instances, the modulator of calcineurin/NFAT signaling is used alone, i.e. in the absence of other growth factors, cytokines, etc, to promote or antagonize β cell development. In some instances, the calcineurin/N FAT signaling modulatory agent is used in combination with other agents, e.g. growth factors, cytokines, intracellular proteins, RNAs, small molecules, known in the art to modulate β cell development, proliferation, or function. For example, an agent that promotes calcineurin/NFAT signaling may be used in combination with another agent known in the art to enhance the rate of β cell maturation, the number of β cells produced, or the production of insulin. Non-limiting examples of agents known in the art to promote β cell maturation, proliferation, and/or function include PDGF (Chen et al. (2011) PDGF signalling controls age-dependent proliferation in pancreatic β-cells. Nature 478(7369):349-55), Wnts (Rulifson et al. (2007) Wnt signaling regulates pancreatic beta cell proliferation. Proc Natl Acad Sci USA. 104(15):6247-52), incretin and agents that promote incretin activity, e.g. Skp2 (Tschen et al. (2011) Skp2 is required for incretin hormone-mediated β-cell proliferation. Mol Endocrinol. 25(12):2134-43), and glucokinase activators and/or exendin-4 (Nakamura et al. (2012) Control of beta cell function and proliferation in mice stimulated by small-molecule glucokinase activator under various conditions. Diabetologia 55(6):1745-54), the full disclosures of which are incorporated herein by reference.

### In Vitro Applications

The subject methods may be used to modulate β cell development from progenitor cells in vitro, for example to produce an enriched population of β cells, i.e. a population of cells that is enriched for mature β cells, for research or for transplantation into an individual.

Cells may be from any mammalian species, e.g. murine, rodent, canine, feline, equine, bovine, ovine, primate, human, etc., or derived from cells of any mammalian species. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

If the cells are primary cells, they may be harvest from an individual by any convenient method. For example, cells, e.g. blood cells, e.g. leukocytes, may be harvested by apheresis, leukocytapheresis, density gradient separation, etc. As another example, cells, e.g. skin, muscle, bone marrow, spleen, liver, pancreas, lung, intestine, stomach, nervous system tissue, etc. may be harvested by biopsy. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The modulator of calcineurin/NFAT signaling is provided to the progenitor cells in culture for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The modulator may be provided to the progenitor cells one or more times, e.g. one time, twice, three times, or more than three times, and the cells allowed to incubate with the agent for some amount of time following each contacting event e.g. 16-24 hours, after which time the media is replaced with fresh media and the cells are cultured further.

Contacting the progenitor cells with the modulator of calcineurin/NFAT signaling may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which β cells or their progenitors are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Exemplary conditions may be found in the working examples provided below.

In some instances, the population of mature β cells may be expanded, e.g. by providing factors to the mature 6 cells that promote 6 cell proliferation. In other words, the number of differentiated β cells in the composition may be increased by promoting their proliferation. As developed in the working examples below, a number of proteins are disclosed herein that act in β cells to promote proliferation, including, for example, cyclin A2, cyclin D2, and FoxM1. As such, agents that promote the activity of these proteins find use in promoting the expansion of the mature β cells in vitro. Other non-limiting examples include PDGF, Wnts, incretin and agents that promote incretin activity, and glucokinase activators and/or exendin-4, as discussed above.

In some instances, the population of cells may be enriched for mature β cells by separating the mature β cells from the remaining population. Separation of β cells typically relies upon the expression of a selectable marker. By a “selectable marker” it is meant an agent that can be used to select cells, e.g. a marker that is ectopically provided, or a marker that is endogenously expressed by and specific for mature β cells, e.g. as described herein. In some instances, the selection may be positive selection; that is, the mature 6 cells are isolated from a population, e.g. to create an enriched population of mature β cells. In other instances, the selection may be negative selection; that is, the population is isolated away from the mature β cells, e.g. to create an enriched population of cells that do not comprise the mature β cells.

Separation may be by any convenient separation technique appropriate for the selectable marker used. For example, if a fluorescent marker has been introduced into the cells, e.g. as progenitor cells, or during the course of differentiation, cells may be separated by fluorescence activated cell sorting. Alternatively, known markers of mature β cells, e.g. as described herein, may be used. Mature β cells may be separated from the heterogeneous population by affinity separation techniques, e.g. magnetic separation, affinity chromatography, “panning” with an affinity reagent attached to a solid matrix, flow cytometry, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the mature β cells.

For example, to separate the mature β cells by affinity separation techniques, cells that are not mature β cells may be depleted from the population by contacting the population with affinity reagents that specifically recognize and selectively bind markers that are not expressed on mature β cells. Additionally or alternatively, positive selection and separation may be performed using by contacting the population with affinity reagents that specifically recognize and selectively bind markers associated with mature β cells. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody. In some embodiments, the affinity reagent may be a specific receptor or ligand for a protein expressed on the cell surface e.g. a peptide ligand and receptor; effector and receptor molecules; a T-cell receptor, and the like. In some embodiments, multiple affinity reagents may be used. Markers and flow cytometry gating strategies that may be used to selectively purify mature β cells from other cells produced from progenitor cells by the subject methods are well known in the art; see, for example Hald J, et al. ((2012) Pancreatic islet and progenitor cell surface markers with cell sorting potential. Diabetologia. 55(1):154-65); Szabat M, et al. ((2011) Kinetics and genomic profiling of adult human and mouse β-cell maturation. Islets. 3(4):175-87); and Köhler M, et al. ((2012) One-step purification of functional human and rat pancreatic alpha cells. Integr Biol (Camb). 4(2):209-19), the disclosures of which are incorporated herein in their entirety by reference.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The population of cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent, i.e. the mature β cells, are selected for by any convenient affinity separation technique, e.g. as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Cell compositions that are highly enriched for mature β cells are achieved in this manner. By “highly enriched”, it is meant that the mature β cells will be 70% or more, 75% or more, 80% or more, 85% or more, 90% or more of the cell composition, for example, about 95% or more, or 98% or more of the cell composition. In other words, the composition may be a substantially pure composition of mature β cells.

Mature β cells produced by the methods described herein may be used immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The mature β cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Compositions of mature β cells that have been prepared by the subject methods find many uses. For example, such compositions may be used in research, e.g. to develop a better understanding of the nature of pancreatic diseases, or to screen candidate agents for those that may be developed to treat pancreatic disease, as described in greater detail below. As another example, such compositions may be transplanted to a subject for purposes such as to treat disease, e.g. diabetes. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals, e.g. murine, lagomorpha, etc. may be used for experimental investigations.

In some cases, the mature β cells may be genetically altered prior to transplanting to the individual, in order to introduce genes useful in the cell, e.g. repair of a genetic defect in an individual, to provide a selectable or traceable marker, etc. The mature β cells may also be genetically modified to enhance survival, control proliferation, and the like. Cells may be genetically altering by transfection or transduction of the progenitor cell or the mature β cell with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the β cells, various techniques may be employed. The genome of the cells may be restricted and used with or without amplification. The polymerase chain reaction; gel electrophoresis; restriction analysis; Southern, Northern, and Western blots; sequencing; or the like, may all be employed. Various tests in vitro and in vivo may be employed to ensure that mature β cell phenotypes have been maintained.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the pancreas, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint or organ; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

The number of administrations of treatment to a subject may vary. Introducing the mature β cells into the subject may be a one-time event; but in certain situations, such treatment may elicit improvement for a limited period of time and require an on-going series of repeated treatments. In other situations, multiple administrations of the mature β cells may be required before an effect is observed. The exact protocols depend upon the disease or condition, the stage of the disease and parameters of the individual subject being treated.

### In Vivo Applications

The subject methods may also be used to modulate β cell development from progenitor cells in vivo, for example to augment the number or function of β cells in an individual, e.g. an individual with diabetes, or, for example, to suppress the expansion of β cells in an individual, e.g. an individual with insulanemia, In these in vivo embodiments, the modulator of calcineurin/N FAT signaling is administered directly to the individual. A calcineurin/NFAT signaling modulator may be administered by any of a number of well-known methods in the art and described below for the administration of peptides, small molecules and nucleic acids to a subject.

As discussed above, the modulator of calcineurin/NFAT signaling is typically administered in an effective amount. The amount administered varies depending upon the goal of the administration, the health and physical condition of the individual to be treated, age, the taxonomic group of individual to be treated (e.g., human, non-human primate, primate, etc.), the degree of resolution desired, the formulation of the calcineurin/NFAT signaling modulator composition, the treating clinician's assessment of the medical situation, and other relevant factors. It is expected that the amount will fall in a relatively broad range that can be determined through routine trials. For example, the amount of modulator of calcineurin/NFAT signaling employed to promote β cell development is not more than about the amount that could otherwise be irreversibly toxic to the subject (i.e., maximum tolerated dose). In other cases the amount is around or even well below the toxic threshold, but still in an immunoeffective concentration range, or even as low as threshold dose.

Individual doses are typically not less than an amount required to produce a measurable effect on the subject, and may be determined based on the pharmacokinetics and pharmacology for absorption, distribution, metabolism, and excretion (“ADME”) of the calcineurin/NFAT signaling modulator or of its by-products, and thus based on the disposition of the composition within the subject. This includes consideration of the route of administration as well as dosage amount, which can be adjusted for topical (applied directly where action is desired for mainly a local effect), enteral (applied via digestive tract for systemic or local effects when retained in part of the digestive tract), or parenteral (applied by routes other than the digestive tract for systemic or local effects) applications. For instance, administration of the modulator of calcineurin/N FAT signaling may be via injection, e.g. intravenous, intramuscular, or intrapancreatic injection, or a combination thereof.

The modulator of calcineurin/N FAT signaling may be administered by infusion or by local injection, e.g. by infusion at a rate of about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m²/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration (e.g, by infusion) can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove β cells, e.g. in the case of β cell hypertrophy. The modulator of calcineurin/NFAT signaling can also be administered as part of a combination therapy, in which at least one of an immunotherapy, a diabetes therapy, a cancer therapy, etc. also is administered to the subject (as described in greater detail below).

Disposition of the modulator of calcineurin/NFAT signaling and its corresponding biological activity within a subject is typically gauged against the fraction of modulator of calcineurin/NFAT signaling present at a target of interest. For example, a modulator of calcineurin/NFAT signaling once administered can accumulate with a glycoconjugate or other biological target that concentrates the material in cancer cells and cancerous tissue. Thus dosing regimens in which the modulator of calcineurin/N FAT signaling is administered so as to accumulate in a target of interest over time can be part of a strategy to allow for lower individual doses. This can also mean that, for example, the dose of calcineurin/NFAT signaling modulator that are cleared more slowly in vivo can be lowered relative to the effective concentration calculated from in vitro assays (e.g., effective amount in vitro approximates mM concentration, versus less than mM concentrations in vivo).

As an example, the effective amount of a dose or dosing regimen can be gauged from the IC₅₀ of a given antagonist of calcineurin/NFAT signaling for inhibiting β cell differentiation. By “IC₅₀” is intended the concentration of a drug required for 50% inhibition in vitro. Alternatively, the effective amount can be gauged from the EC₅₀ of a given calcineurin/NFAT signaling modulator concentration. By “EC₅₀” is intended the plasma concentration required for obtaining 50% of a maximum effect in vivo. In related embodiments, dosage may also be determined based on ED₅₀ (effective dosage).

In general, with respect to the modulator of calcineurin/NFAT signaling of the present disclosure, an effective amount is usually not more than 200× the calculated IC₅₀. Typically, the amount of a modulator of calcineurin/N FAT signaling that is administered is less than about 200×, less than about 150×, less than about 100× and many embodiments less than about 75×, less than about 60×, 50×, 45×, 40×, 35×, 30×, 25×, 20×, 15×, 10× and even less than about 8× or 2× than the calculated IC₅₀. In one embodiment, the effective amount is about 1× to 50× of the calculated IC₅₀, and sometimes about 2× to 40×, about 3× to 30× or about 4× to 20× of the calculated IC₅₀. In other embodiments, the effective amount is the same as the calculated IC₅₀, and in certain embodiments the effective amount is an amount that is more than the calculated IC₅₀.

An effect amount may not be more than 100× the calculated EC₅₀. For instance, the amount of a modulator of calcineurin/N FAT signaling that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC₅₀. The effective amount may be about 1× to 30× of the calculated EC₅₀, and sometimes about 1× to 20×, or about 1× to 10× of the calculated EC₅₀. The effective amount may also be the same as the calculated EC₅₀ or more than the calculated EC₅₀. The EC₅₀ can be calculated by modulating β cell proliferation in vitro. The procedure can be carry out by methods known in the art or as described in the examples below.

Effective amounts of dose and/or dose regimen can readily be determined empirically from assays, from safety and escalation and dose range trials, individual clinician-patient relationships, as well as in vitro and in vivo assays such as those described herein and illustrated in the Experimental section, below. For example, if a concentration used for carrying out the subject method in mice ranges from about 1 mg/kg to about 25 mg/kg based on the body weight of the mice, an example of a concentration of the calcineurin/N FAT signaling modulator that can be employed in human may range about 0.083 mg/kg to about 2.08 mg/kg. Other dosage may be determined from experiments with animal models using methods known in the art (Reagan-Shaw et al. (2007) The FASEB Journal 22:659-661).

The calcineurin/NFAT signaling modulator can be incorporated into a variety of formulations. More particularly, the calcineurin/NFAT signaling modulator may be formulated into pharmaceutical compositions by combination with appropriate pharmaceutically acceptable carriers or diluents.

Pharmaceutical preparations are compositions that include one or more calcineurin/N FAT signaling modulator present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the calcineurin/NFAT signaling modulator can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For inclusion in a medicament, the calcineurin/NFAT signaling modulator may be obtained from a suitable commercial source. As a general proposition, the total pharmaceutically effective amount of the calcineurin/N FAT signaling modulator administered parenterally per dose will be in a range that can be measured by a dose response curve.

Calcineurin/N FAT signaling modulator-based therapies, i.e. preparations of calcineurin/NFAT signaling modulator(s) to be used for therapeutic administration, may be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The calcineurin/N FAT signaling modulator-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection. Alternatively, the calcineurin/NFAT signaling modulator may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The modulator of calcineurin/NFAT signaling may be provided in addition to other agents. For example, in methods of inhibiting β cell development, e.g. to treat insulinoma, mixed endocrine tumor, or acquired states of β cell overgrowth, a calcineurin/NFAT signaling antagonist may be coadministered with other known cancer therapies. As another example, in methods of promoting β cell development, e.g. to treat diabetes, a calcineurin/NFAT signaling agonist may be coadministered with other known diabetes therapies.

### Utility

The subject methods and compositions find many uses. For example, the subject methods may be used to treat diseases associated with defective β cell maturation or β cell dysfunction. The terms “treatment”, “treating” and the like are used herein to generally mean obtaining a desired pharmacologic and/or physiologic effect in an individual. The terms “individual,” “subject,” “host,” and “patient,” are used interchangeably herein and refer to any mammalian subject for whom diagnosis, treatment, or therapy is desired, particularly humans. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

For example, subject methods comprising promoting calcineurin/NFAT signaling may be used to treat disorders associated with a decrease in β cell mass or function, e.g. by promoting β cell development in vitro/ex vivo to generate mature β cells ex vivo for replacement therapy; or to promote β cell development in vivo to regenerate β cell mass. Examples of such diseases and disorders that may be treated using such methods include those that are associated with insulin resistance or with the reduced production of insulin, for example, diabetes.

Diabetes is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”). Type 1 diabetes, also known as insulin dependent diabetes mellitus (IDDM), results from the destruction or dysfunction of β cells by the cells of the immune system. Symptoms include polyuria (frequent urination), polydipsia (increased thirst), polyphagia (increased hunger), and weight loss. T1D is fatal unless treated with insulin and must be continued indefinitely, although many people who develop the disease are otherwise healthy and treatment need not significantly impair normal activities. Exercising regularly, eating healthy foods and monitoring blood sugar may also be recommended. Other medications may be prescribed as well, including one or more of the following: medications to slow the movement of food through the stomach (e.g. pramlintide), high blood pressure medications, cholesterol-lowering drugs. Type 2 diabetes, also known as non-insulin dependent diabetes mellitus (NIDDM), is associated with a gradual decline in β cell function and numbers over time, as the β cells develop resistance to insulin. As a result, in T2D the pancreas does not make enough insulin to keep blood glucose levels normal. Symptoms include hyperglycemia (high blood sugar), diabetic ketoacidosis (increased ketones in urine), and hyperosmolar hyperglycemic nonketotic syndrome. Therapy may include blood sugar monitoring; healthy eating; regular exercise; diabetes medication that lowers glucose production (e.g. metformin, sitagliptin, saxagliptin, repaglinide, nateglinide, exenatide, liraglutide), that stimulates the pancreas to produce and release more insulin (e.g. glipizide, glyburide, glimepiride), and/or that blocks the action of enzymes that break down carbohydrates or make tissues more sensitive to insulin (e.g. pioglitazone); and insulin therapy.

Other disorders associated with insulin resistance that may likewise be treated using the subject methods include, for example, diabetic angiopathy, atherosclerosis, diabetic nephropathy, diabetic neuropathy, and diabetic ocular complications such as retinopathy, cataract formation and glaucoma, as well as glucocorticoid induced insulin resistance, dyslipidemia, polycysitic ovarian syndrome, obesity, hyperglycemia, hyperlipidemia, hypercholesterolemia, hypertriglyceridemia, hyperinsulinemia, and hypertension. Methods of identifying an individual having one of these disorders are well-known in the art, as are methods for detecting the symptoms of these disorders and the relief from the symptoms upon treatment with the calcineurin/NFAT agonist.

As another example, subject methods comprising antagonizing calcinuerin/N FAT signaling may be used to treat disorders associated with increased β-cell mass or β cell hyperactivity, for example to inhibit β cell development and/or function in vivo to prevent further hypertrophy or hyperactivity. Examples of diseases that may be treated in this way include congenital or acquired hyperinsulinism, nesidioblastosis following bariatric surgery, insulinomas and other neuroendocrine cancers.

Hyperinsulinism refers to an above-normal level of insulin in the blood of a person or animal. In normal children and adults, insulin secretion should be minimal when blood glucose levels fall below 70 mg/dL (3.9 mM). Insulin levels above 3 μU/mL are inappropriate when the glucose level is below 50 mg/dL (2.8 mM), and may indicate hyperinsulinism as the cause of the hypoglycemia. There are many forms of hyperinsulinemia caused by various types of insulin excess. For example, congenital hyperinsulinism occurs in infants and young children, and may be the result of genetic abnormalities, the intrauterine environment, errors of morphogenesis, infection, or a chromosomal abnormality. In adults, severe hyperinsulinemia is often due to an insulinoma (an insulin-secreting tumor of the pancreas, discussed further below). Hyperinsulinemia may also be caused by nesidioblastosis, e.g. after bariatric (e.g. gastric bypass) surgery. Treatment of hyperinsulinism depends on the cause and the severity of the hyperinsulinism, and may include surgical removal of the source of insulin, or a drug such as diazoxide or octreotide that reduces insulin secretion.

Insulinoma refers to a rare tumor derived from β cells. Insulin secretion by insulinomas is not properly regulated by glucose. As such, tumors continue to secrete insulin, causing glucose levels to fall further than normal. The diagnosis of an insulinoma is usually made biochemically with low blood glucose, elevated insulin, proinsulin and C-peptide levels and confirmed by localizing the tumor with medical imaging or angiography. The definitive treatment is surgery. Insulinomas are usually benign and not malignant, but may be medically significant and even life-threatening due to recurrent and prolonged attacks of hypoglycemia. Insulinomas and other neuroendocrine cancers would therefore benefit from treatment using the subject methods.

Methods for modulating β cell development by providing a calcineurin/NFAT signaling modulator may also be applied to studying β cell development, proliferation, and/or function in vitro. For example, the methods described above provide a useful system for screening candidate agents for activity modulating β cell development. To that end, it has been shown that calcineurin/NFAT signaling modulates β cell development. Accordingly, screening candidate agents to identify those that promote calcineurin/NFAT activity should identify agents that find use in promoting β cell development, whereas screening candidate agents to identify those that inhibit calcineurin/NFAT activity should identify agents that find use in inhibiting β cell development. In one example of such a screen, progenitor cells are contacted with a candidate agent, and one or more cellular parameters reflective of the activity of the calcineurin/NFAT signaling pathway is measured. The measured cellular parameter(s) are compared to the cellular parameter(s) measured in progenitor cells not contacted with the candidate agent. An increase in calcineurin/N FAT activity indicates that the candidate agent will promote β cell development.

As another example, screening candidate agents to identify those that promote β cell development in calcineurin- or NFAT-knockout cells or from progenitor cells in the presence of an inhibitor of calcineurin/NFAT signaling should identify signaling pathways other than the calcineurin/NFAT signaling pathway that promote β cell development and that can be targeted for drug development, whereas screening candidate agents to identify those that inhibit β cell development from progenitor cells in the presence of an activator of calcineurin/NFAT signaling should identify signaling pathways other than the calcineurin/NFAT signaling pathway that inhibit β cell development and that can be targeted for drug development. In one example of such a screen, calcineurin-deficient progenitor cells, e.g. as described in the working examples below or as known in the art, are contacted with a candidate agent under conditions that normally promote β cell development, and one or more cellular parameters reflective of β cell maturation is measured. The measured cellular parameter(s) are compared to the cellular parameter(s) measured in calcineurin-deficient progenitor cells not contacted with the candidate agent. An increase in mature β cells in the culture indicates that the candidate agent targets a protein that promotes β cell development independent of calcineurin.

Cellular parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. As will be readily apparent to the ordinarily skilled artisan, a number of output cellular parameters may be quantified when screening for agents that modulate the activity of calcineurin/NFAT, or that modulate the development of β cells. For example, the localization of NFAT to the nucleus may be assessed by, e.g., immunohistochemistry; the binding of NFAT to chromatin may be assessed by, e.g. EMSA or Chromatin IP (ChIP). The expression of NFAT target genes may be measured, e.g. by Northern blot, RT-PCR, Western blot, etc. The expression of an ectopically-provided reporter downstream of an NFAT-specific promoter may be measured. Parameters reflective of the extent of β cell maturation in the culture may be measured, e.g. the number of dense core granules (DCGs) per cell, or the number of cells having a density of DCGs comparable to mature β cells, the amount of insulin, C-peptide, or IAPP produced by the cells, etc. Any convenient parameter that reflects the activity of calcineurin/NFAT signaling and/or β cell maturation may be measured. In some instances, multiple parameters are measured.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Nucleic acids may be provided as vectors, viruses, or any other convenient method known in the art or described elsewhere herein.

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

Because the candidate polypeptide agent is being assayed for its ability to inhibit the activity of an intracellular protein, the polypeptide may be myristoylated, or comprise the polypeptide sequences of interest fused to a polypeptide permeant domain.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of biochemical or cell-based reactions, usually in conjunction with biochemical reactions or cells not contacted with the agent. The change in parameters in response to the agent is measured, and the result evaluated by comparison to reference samples, e.g. in the presence and absence of the agent, obtained with other agents, etc.

The agents are conveniently added in solution, or readily soluble form, to the cell-free reaction or medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The subject compositions comprising mature β cells prepared by the subject methods may also be used as a basic research or drug discovery tool, for example to evaluate the phenotype of a genetic disease, e.g. to better understand the etiology of the disease, to identify target proteins for therapeutic treatment, to identify candidate agents with disease-modifying activity, i.e. an activity in modulating the survival or function of β cells in a subject suffering from a pancreatic disease or disorder, e.g. to identify an agent that will be efficacious in treating the subject.

### Reagents and Kits

Also provided are reagents and kits thereof for practicing one or more of the subject methods. The subject reagents and kits thereof may vary greatly. Reagents and devices of interest include those mentioned above with respect to the methods of promoting or inhibiting β cell development, treating disorders such as diabetes or insulinoma that are associated with defects in β-cell function, and screening candidate agents for the ability to treat disorders associated with defects in β cell function by modulating calcineurin/N FAT signaling to modulate β cell development, proliferation, and function. Reagents may include one or more of the following: one or more agents that is an agonist or antagonist of calcineurin/N FAT signaling; buffer or pharmaceutical excipient into which the agent(s) may be dissolved for contacting cells or administering to an individual; and cells, media, and reagents as discussed above or in the working examples below for cell-based screens for candidate agents for modulating β cell development.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

To meet host metabolic demands after birth, organs like pancreatic islets increase their physiological function and mass. Compared to fetal islet development, however, little is known about mechanisms governing neonatal islet maturation and expansion. Here we demonstrate calcineurin/Nuclear Factor of Activated T-cells (Cn/N FAT) signaling regulates both β-cell maturation and proliferation in neonatal mice and humans. Inactivation of the gene encoding the calcineurin phosphatase regulatory subunit, calcineurin b1 (Cnb1), in mouse islets resulted in defective dense core granule biogenesis, impaired insulin secretion, and reduced neonatal β-cell proliferation and mass, culminating in lethal, early-onset diabetes. β-cells lacking Cnb1 failed to express genes required for insulin storage and secretion, as well as neonatal replication. In contrast, exposure of islets to glucokinase activator stimulated Cn-dependent expression of these genes. Tacrolimus, a calcineurin inhibitor and widely used immunosuppressant, reduces human β-cell secretion and promotes diabetes, toxicities without a clear molecular basis. Exposure of mouse and human islets to tacrolimus reduced expression of genes encoding factors essential for insulin dense core granule formation and secretion, and neonatal β-cell proliferation consistent with our genetic studies. Chromatin immunoprecipitation and other molecular studies revealed these genes as novel, direct NFAT targets in neonatal mouse and human islets. Thus, calcineurin/NFAT signaling coordinately regulates factors that govern β-cell maturation and proliferation, revealing unique models for the pathogenesis and therapy of diabetes mellitus and diverse human islet diseases.

Defects in β-cell function and number underlie many human diseases, most notably diabetes mellitus. Emerging strategies to achieve replacement or regeneration of pancreatic β-cells rely on knowledge about β-cell development and growth. β-cells form in the embryonic pancreas and understanding of the molecular and cellular basis for this early stage of development has grown in recent years (Seymour and Sander (2011). Historical perspective: beginnings of the beta-cell: current perspectives in beta-cell development. Diabetes 60, 364-376). After birth of mice, humans and other animals, normal β-cell development continues, leading to achievement of two crucial milestones. First, enhancement of -glucose sensing, insulin production per cell, and increase of insulin-containing dense core secretory granules, result in the maturation of β-cell stimulus-secretion coupling (Bencosme (1955) The histogenesis and cytology of the pancreatic islets in the rabbit. American Journal of Anatomy 96, 103-151; Bruin et al. (2008) Fetal and neonatal nicotine exposure in Wistar rats causes progressive pancreatic mitochondrial damage and beta cell dysfunction. PLoS ONE 3, e3371; Kim et al. (2006) Dense-core secretory granule biogenesis. Physiology (Bethesda) 21, 124-133; Rozzo et al. (2009) Exocytosis of insulin: in vivo maturation of mouse endocrine pancreas. Ann. N.Y. Acad. Sci 1152, 53-62). Second, proliferation in neonatal mice and human islets leads to expansion and establishment of appropriate β-cell mass (Georgia and Bhushan (2004) Beta cell replication is the primary mechanism for maintaining postnatal beta cell mass. J. Clin. Invest 114, 963-968; Meier et al. (2008) Beta-cell replication is the primary mechanism subserving the postnatal expansion of beta-cell mass in humans. Diabetes 57, 1584-1594; Teta et al. (2005) Very slow turnover of beta-cells in aged adult mice. Diabetes 54, 2557-2567). Defective β-cell maturation or growth promotes pathogenesis of diabetes and other diseases (Kapoor et al. (2009) Advances in the diagnosis and management of hyperinsulinemic hypoglycemia. Nat Clin Pract Endocrinol Metab 5, 101-112; McKnight et al. (2010) Deconstructing pancreas development to reconstruct human islets from pluripotent stem cells. Cell Stem Cell 6, 300-308). Despite the importance of β-cell functional maturation and expansion to human health, little is known about the mechanisms controlling and coordinating these crucial steps of β-cell development.

To achieve effective glucose sensing and insulin secretion, β-cells enhance expression of genes encoding hallmark factors, including Preproinsulin, glucose transporters (like Glut2 and Glucokinase), and the transcription factor Pdx1 during the first three postnatal weeks in mice (Aguayo-Mazzucato et al. (2011) Mafa expression enhances glucose-responsive insulin secretion in neonatal rat beta cells. Diabetologia 54, 583-593; Jermendy et al. (2011) Rat neonatal beta cells lack the specialised metabolic phenotype of mature beta cells. Diabetologia 54, 594-604). In β-cells, processed insulin is stored in secretory vesicles that have an electron-dense core in ultrastructural analysis (Kim et al. (2006), supra) These intracellular dense core granules (DCGs) harbor several principal protein components including the hormones insulin and islet amyloid polypeptide (IAPP), granins encoded by chromogranin A (ChgA) and chromogranin B (ChgB), and transmembrane proteins like IA2 (also called ICA152) (Kim et al. (2006) supra; Suckale and Solimena (2010) The insulin secretory granule as a signaling hub. Trends Endocrinol. Metab 21, 599-609). Prior studies suggest IA2 is an important regulator of DCG formation and insulin secretion, via linked transcriptional and post-transcriptional mechanisms (Harashima et al. (2005) The dense core transmembrane vesicle protein IA-2 is a regulator of vesicle number and insulin secretion. Proc. Natl. Acad. Sci. U.S.A 102, 8704-8709; Mziaut et al. (2006) Synergy of glucose and growth hormone signalling in islet cells through ICA512 and STATS. Nat. Cell Biol 8, 435-445; Saeki et al. (2002) Targeted disruption of the protein tyrosine phosphatase-like molecule IA-2 results in alterations in glucose tolerance tests and insulin secretion. Diabetes 51, 1842-1850). For example, studies of immortalized β-cell lines suggests that depolarization stimulates Ca²⁺-dependent cleavage of IA2 to induce transcription of genes encoding Insulin, Prohormone convertase 1/3, and IA2 itself (Mziaut et al. (2006) supra; Trajkovski et al. (2004) Nuclear translocation of an ICA512 cytosolic fragment couples granule exocytosis and insulin expression in {beta}-cells. J. Cell Biol 167, 1063-1074). Other studies suggest that IA2 is necessary and sufficient for regulating DCG number in the mouse MIN6 β-cell line (Harashima et al. (2005) supra). These in vitro studies suggest how activity-dependent regulation maintains DCGs in adult β-cells, but it remains unclear how Ca²⁺-dependent β-cell pathways might regulate transcription of hallmark DCG components like granins and IAPP, or how β-cell DCG formation is regulated in vivo within mice, as well as humans.

In concert with their maturation, β-cells replicate, and this β-cell expansion is postulated to modulate diabetes susceptibility (Butler et al. (2007). The replication of beta cells in normal physiology, in disease and for therapy. Nat Clin Pract Endocrinol Metab 3, 758-768). Studies have identified regulators required for neonatal β-cell replication and establishment of β-cell mass, including cyclin dependent kinase 4 (Cdk4) and D type cyclins (Georgia and Bhushan (2004), supra; Kushner et al. (2005) Cyclins D2 and D1 are essential for postnatal pancreatic beta-cell growth. Mol. Cell. Biol 25, 3752-3762; Rane et al. (1999) Loss of Cdk4 expression causes insulin-deficient diabetes and Cdk4 activation results in beta-islet cell hyperplasia. Nat. Genet. 22, 44-52), the transcription factor FoxM1 (Zhang et al. (2006) The FoxM1 transcription factor is required to maintain pancreatic beta-cell mass. Mol. Endocrinol 20, 1853-1866), and other factors. Islet CyclinD2 (CcnD2) and FoxM1 protein levels are highest in neonatal mice, then decline in adults, indicating that transcription of CcnD2 and FoxM1 may regulate and limit β-cell proliferation, but this possibility has not been previously explored. Moreover, it is unknown if these or other factors regulate physiological neonatal β-cell expansion in humans (Davis et al. (2010) FoxM1 is up-regulated by obesity and stimulates beta-cell proliferation. Mol Endocrinol 24, 1822-34; Heit et al. (2006b) Intrinsic regulators of pancreatic beta-cell proliferation. Annu. Rev. Cell Dev. Biol 22, 311-338; Heit (2007) Calcineurin/N FAT signaling in the beta-cell: From diabetes to new therapeutics. Bioessays 29, 1011-1021).

Glucose signaling is a physiological regulator of β-cell functional maturation and proliferation. Glucokinase is a crucial regulator of β-cell glucose metabolism and prior studies demonstrate that glucokinase activation stimulates Ca²⁺ transients and depolarization, which in turn enhance β-cell production of insulin (Lawrence et al. (2001) Regulation of insulin gene transcription by a Ca(2+)-responsive pathway involving calcineurin and nuclear factor of activated T cells. Mol. Endocrinol 15, 1758-1767), insulin secretion (Grimsby et al. (2003) Allosteric activators of glucokinase: potential role in diabetes therapy. Science 301, 370-3) and proliferation (Pechhold et al. (2009) Blood glucose levels regulate pancreatic beta-cell proliferation during experimentally-induced and spontaneous autoimmune diabetes in mice. PLoS ONE 4, e4827; Porat et al. (2011) Control of pancreatic β cell regeneration by glucose metabolism. Cell Metab. 13, 440-449; Salpeter et al. (2011) Glucose regulates cyclin D2 expression in quiescent and replicating pancreatic β-cells through glycolysis and calcium channels. Endocrinology 152, 2589-2598). Glucokinase mRNA and activity increase during the period of postnatal β-cell growth and maturation (Aguayo-Mazzucato et al. (2011) supra; Rozzo et al. (2009) Exocytosis of insulin: in vivo maturation of mouse endocrine pancreas. Ann. N.Y. Acad. Sci 1152, 53-62; Taniguchi et al. (2000) Immaturity of glucose-induced insulin secretion in fetal rat islets is due to low glucokinase activity. Horm Metab Res 32, 97-102); thus glucokinase regulated depolarization and Ca²⁺ signaling may be physiological regulators of pathways governing β-cell proliferation and functional specialization.

The calcineurin/Nuclear Factor of Activated T-cells (Cn/N FAT) pathway regulates gene transcription to coordinate proliferation, survival and differentiation of diverse cell types, including lymphocytes and neurons (Wu et al. (2007) NFAT signaling and the invention of vertebrates. Trends Cell Biol 17, 251-260). Calcineurin is a Ca²⁺-activated serine/threonine phosphatase required for activation of the NFATc family of transcription factors (NFATc1-c4). With sustained rises in intracellular Ca²⁺, calcineurin activation leads to dephosphorylation of NFATc proteins and other substrates (Crabtree and Olson (2002) NFAT signaling: choreographing the social lives of cells. Cell 109 Suppl, S67-79), a step permitting NFATc nuclear translocation and regulation of gene transcription. A role for Cn/NFAT in human β-cell function has been indirectly inferred from the striking observation that 10-30% of patients requiring immunosuppression with calcineurin inhibitors like tacrolimus (FK506) develop diabetes mellitus (Montori et al. (2002) Posttransplantation diabetes: a systematic review of the literature. Diabetes Care 25, 583-592; Oetjen et al. (2003) Inhibition of human insulin gene transcription by the immunosuppressive drugs cyclosporin A and tacrolimus in primary, mature islets of transgenic mice. Mol. Pharmacol 63, 1289-1295). A role for Cn/NFAT signaling in adult mouse pancreatic β-cell proliferation was previously reported (Heit et al. (2006a) Calcineurin/NFAT signalling regulates pancreatic beta-cell growth and function. Nature 443, 345-349). Conditional genetic abrogation of Cn/NFAT in that study, however, resulted in an adult, non-lethal phenotype where β-cell development could not be investigated. β-cell proliferation and mass from birth through 8 weeks of age in ‘pCnb1KO’ mice was indistinguishable from littermate controls and by 10 weeks, these mice developed mild hyperglycemia accompanied by a reduced β-cell mass. However, a role for Cn/NFAT in insulin secretion was not investigated, nor any possible functions for this pathway in human islet development and disease.

Here we used conditional genetics to inactivate Cnb1 in neonatal islets, revealing a requirement for Cn/NFAT signaling in neonatal β-cell development including DCG biogenesis, functional maturation and mass establishment. Additionally, unprecedented studies of islets from young human subjects show that Cn/NFAT-regulated mechanisms governing DCG formation and β-cell replication are conserved in humans. The changes in human β-cell gene expression and impaired proliferation in human islets exposed to FK506 described here, also unveil new molecular and cellular rationales for the long-standing clinical observation that calcineurin inhibitors promote diabetes mellitus.

### Materials and Methods

Animals. Mice harboring the Cnb1^{f} or Cnb1^{Δ} alleles, have exons 3 to 5 flanked by loxP sites or are excised, respectively (Winslow et al. (2006) The calcineurin phosphatase complex modulates immunogenic B cell responses. Immunity 24, 141-152). Transgenic Ngn3-Cre and Pdx1-Cre mice were provided by the Leiter and Melton laboratories, respectively and previously described (Gu et al. (2002) Direct evidence for the pancreatic lineage: NGN3+ cells are islet progenitors and are distinct from duct progenitors. Development 129, 2447-57; Schonhoff et al. (2004) Neurogenin 3-expressing progenitor cells in the gastrointestinal tract differentiate into both endocrine and non-endocrine cell types. Dev. Biol 270, 443-454). These strains were crossed to generate Ngn3-Cre; Cnb1^{Δ/f} (nCnb1KO) mice or Pdx1-Cre; Cnb1^{Δ/f} (pCnb1KO) and their littermate controls on a mixed 129/Sv and C57/BL6 genetic background. Mice were genotyped routinely by PCR from tail genomic DNA for the deleted, floxed and WT alleles of Cnb1 as well as the Cre transgenes as previously described. Inbred C57/BL6 mice for ChIP analyses were purchased from Charles River. MIP-EGFP mice were obtained from M. Hara and G. Bell (University of Chicago, Chicago, Ill.). All animals were maintained on a 12-hour light/dark cycle with ad libitum access to water and chow. All handling, experimentation and methods were in accordance with the Institutional Animal Care and Use Committee (IACUC) of Stanford University.

Quantitative Real-time RT-PCR. Mouse islets were isolated by standard collagenase pancreatic perfusion as previously described (Heit et al. (2006a), supra). Total islet mRNA was then isolated using the RNeasy Microkit (QIAGEN) according to the manufacturer's instructions and RNA amount and purity were assessed by Nanodrop spectrophotometry. Any remaining contaminant DNA was removed by treating samples with 1 unit of RNAse-free DNAse (Fermentas). Next, cDNA was prepared from 750 ng of total islet RNA using the RETROscript kit (Ambion) and analyzed by quantitative real-time PCR (QPCR) using TaqMan Universal PCR Master Mix (ABI) and the ABI Prism 7500 detection system.

For culture studies, islets harvested from mice of the indicated genotype were placed in standard media (RPMI 1640 with 4.5 mM glucose containing 10% FBS and 1% pen/strep) at 37° C. and 5% CO2. For some experiments islets were maintained in media containing vehicle (DMSO), glucokinase activator (GKA) R0-28-1675 (10 μM; Axon Ligands) and/or FK506 (10 μM; LC Laboratories) for 72 hours, with media changes every 24 hours. Additionally, β-cells from MIP-EGFP islets were isolated using fluorescence-activated cell sorting as previously described (Sugiyama, T., et al. (2007). Conserved markers of fetal pancreatic epithelium permit prospective isolation of islet progenitor cells by FACS. Proc. Natl. Acad. Sci. U.S.A 104, 175-180) and processed as above for analysis by QPCR. Table 1 below lists the TaqMan probes (ABI) used. Each quantitative analysis was performed in triplicate and islets from 3 to 6 mice of each genotype were independently tested. Data are normalized to β-actin and results are expressed as the mean±S.E.M.

TABLE 1
QPCR TaqMan probes used for mouse and human islet mRNA
expression assays (purchased from Applied Biosystems).
MOUSE

| | |
|---|---|
| Insulin 1 | Mm01259683_g1 |
| Insulin 2 | Mm00731595_gH |
| Glucokinase | Mm00439129_m1 |
| Glut2 | Mm00446224_m1 |
| Hnf4a | Mm00433964_m1 |
| Chromogranin A | Mm00514341_m1 |
| Chromogranin B | Mm00483287_m1 |
| IAPP | Mm00439403_m1 |
| IA2 | Mm00436138_m1 |
| Cdk4 | Mm00726334_s1 |
| CyclinA2 | Mm00438064_m1 |
| CyclinD1 | Mm00432360_m1 |
| CyclinD2 | Mm00438072_m1 |
| FoxM1 | Mm00514924_m1 |

HUMAN

| | |
|---|---|
| INSULIN | Hs00355773_m1 |
| HNF4a | Hs00230853_m1 |
| CHROMOGRANIN A | Hs00900373_m1 |
| CHROMOGRANIN B | Hs001084631_m1 |
| IAPP | Hs00169095_m1 |
| IA2 | Hs00160947_m1 |
| CYCLINA2 | Hs00996788_m1 |
| CYCLIND2 | Hs00153380_m1 |
| FOXM1 | Hs01073586_m1 |

Physiological studies. Random fed glucose levels were measured in ad libitum fed mice from tail vein blood using the Ascensia Contour glucometer. Glucose tolerance tests were performed following a 16-hour fast and blood glucose levels measured immediately before (0) and 15, 30, 45, 60, and 75 min after intraperitoneal (IP) injection of D-glucose (1 g/Kg body weight). For insulin sensitivity studies, random-fed mice were IP injected with insulin (Sigma) at 1 U/kg body weight. Blood glucose levels were taken at indicated times and expressed as a percentage of the initial blood glucose concentration. Both serum insulin and glucagon levels were assessed in P26 mice that were fasted for 4 hours. Following euthanasia, blood was collected by cardiac puncture and serum was isolated after centrifugation. Serum insulin and glucagon levels were measured using the Mouse Insulin Ultrasensitive EIA kit (Alpco) and Glucagon ELISA kit (Alpco), respectively. (Heit et al. (2006a), supra).

Histology, Immunofluorescence, and Immunohistochemistry.

Pancreata were isolated, fixed in 4% paraformaldehyde at 4° C. for 2 hours and washed 3 times in PBS. For paraffin sections, samples were then serially dehydrated in increasing concentrations of ethanol (25, 50, 75, 90, 100%) for 2 minutes at each concentration and then placed in xylenes for 2 hours. The samples were then embedded in paraffin wax blocks and sectioned at a thickness of 6 μm. For cryo sections, samples were cryoprotected in 30% sucrose overnight, embedded in O.C.T. (TissueTek) and 8 μm sections were obtained by cryosection (Leica). All immunohistochemistry was performed on paraffin sections while β-cell mass morphometry was done on cryosections.

The following primary antibodies and dilutions were used: chicken anti-insulin (1:200; Abcam, ab14042), guinea pig (GP) anti-glucagon (1:200; Linco, 4031-01), goat anti-glut2 (1:200; Santa Cruz, sc-7580), rabbit anti-pdx1 (1:100; Chemicon, AB3503), rabbit anti-cyclin A2 (1:100; Thermo Scientific, RB-1548P0), rabbit anti-cyclin D2 (1:100; Santa Cruz, sc-593), rabbit anti-FoxM1 (1:100; Santa Cruz, sc-500), rabbit anti-Cdk4 (1:100; Santa Cruz, sc-260), rabbit anti-chromogranin A (1:100; Immunostar, 20085), rabbit anti-chromogranin B (1:100; Abcam, ab12242), mouse anti-IAPP (1:50; Serotec, CA1126T), mouse anti-IA2 (1:50; Santa Cruz, sc-130570), rabbit anti-Ki67 (1:100; NovoCastra, NCL-Ki67p), rabbit polyclonal anti-cleaved caspase-3 (Asp175) (1:500; Cell Signaling, 9661), mouse monoclonal anti-BRDU (1:100; Sigma). Antigen retrieval was performed for certain markers using either antigen unmasking solution (Vector Laboratories, H-3300) [anti-CcnA2, anti-CcnD2, anti-Cdk4, anti-IAPP, anti-Glut2] or Retrievit-8 target retrieval solution (Biogenex) [anti-activated caspase 3, anti-ChgB, anti-FoxM1, and anti-IA2]. We detected immune complexes with secondary antibodies conjugated with either Alexa 488, Alexa 555 (Molecular Probes) or horseradish peroxidase (Vector Laboratories). All images were collected using the AxioCam microscope equipped with a CCD digital camera (Carl Zeiss) and representative of over 50 islets of a minimum of 3 different mice per genotype.

For measurement of β-cell mass, a minimum of 30 pancreas sections (spanning the entire pancreas) were assessed for at least 3 different mice per genotype. Cross-sectional area of insulin+ cells were measured and normalized to total pancreatic area using Image-Pro Plus analysis software (Media Cybernetics). β-cell mass is expressed in mg, normalized to total pancreas mass.

β-cell proliferation and apoptosis levels were assessed by scoring the number of Ki67⁺ or activated caspase-3⁺ β-cells and expressed as a percentage of the total number of β-cells counted. For each experiment, a minimum of 30 islets/mouse for at least 3 mice/genotype were scored.

Transmission Electron Microscopy (TEM).

For each experiment, roughly 50 size-matched islets were isolated by collagenase perfusion from 3 pre-diabetic P20 nCnb1KO mice and 3 littermate controls as described above. Islets were fixed in Karnovsky's fixative (2% glutaraldehyde [EMS] and 4% paraformaldehyde [EMS] in 0.1 M sodium cacodylate [EMS] pH 7.4) for 1 hour at room temperature (RT). The samples were then cut, postfixed in 1% osmium tetroxide (EMS) for 1 hour at RT, washed 3× with ultrafiltered water and en bloc stained for 2 hrs at RT or left at 4° C. overnight. The samples were then dehydrated in a series of ethanol washes (50%, 70%, 95%) for 15 minutes each at 4° C., where the samples were then allowed to rise to RT. They were then moved to 100% ethanol 2×, followed by Acetonitrile for 15 min.

Samples were infiltrated with EMbed-812 resin (EMS) mixed 1:1 with Acetonitrile for 2 hrs followed by 2 parts EMbed-812 to 1 part Acetonitrile for 2 hours. Finally, they were placed in EMbed-812 for 2 hours, moved into molds and resin filled gelatin capsules and placed into a 65° C. oven overnight. Sections were taken between 75 and 90 nm, picked up on formvar/carbon coated slot grids (EMS) or 100 mesh Cu grids (EMS). Grids were contrast stained for 15 minutes in 1:1 saturated UrAcetate (˜7.7%) to 100% ethanol followed by staining in 0.2% Lead Citrate for 3 to 4 minutes. Samples were observed in the JEOL 1230 TEM at 80 kV and final images were taken using a Gatan Orius digital camera. A total of three experiments were performed, with a minimum of 30 β-cells scored (blinded to genotype) per genotype per experiment.

Islet Insulin Secretion and Islet Insulin Content Measurement.

Islets were isolated and cultured overnight in islet medium (as above) and passed three times through 10 cm petri dishes containing 3 mM glucose islet media and allowed to equilibrate at 37° C. for 1 hour. Five islets were then transferred into each well of an untreated, 24-well plate containing 1 ml of media for each condition (3 mM or 20 mM glucose; 3 mM or 20 mM arginine) and incubated at 37° C. for one hour. Each condition was performed in quadruplicate using islets from at least 3 mice per genotype. Islet DNA content was assessed by nanodrop for each replicate of all conditions and genotypes and media for each condition was removed and levels of secreted insulin were determined using the Mouse Insulin Ultrasensitive EIA kit (Alpco). Values of insulin were then normalized to islet DNA content. For whole islet insulin content, isolated islets were sonicated in 150 μl sonication buffer (150 ml 10 mmol/l Tris HCl, 1 mmol/I EDTA, and 1 mg/ml radioimmunoassay grade BSA (pH 7.0)) for 30 s. Fifty μl were used to extract islet insulin with 100 μl acid ethanol (75 ethanol:2 concentrated HCl:23 H₂O, vol:vol:vol) at 4° C., overnight. The remainder of the sonicate (100 μl) was digested with an equal volume of lysis buffer at 55° C. for 2 hours and used for islet DNA quantification for normalization.

Pancreatic Islet Chromatin Immunoprecipitation (ChIP).

Islets from P20 C57/BL6 mice were isolated (as above) and fixed with 1% formaldehyde for 10 minutes at room temperature. Cross-linking was quenched by the addition of 0.125 M glycine and islets were washed in DPBS. ChIPs were then performed using the EZ-Magna ChIP™ G Chromatin Immunoprecipitation Kit (Millipore), following the manufacturer's protocol. Briefly, following cell and nuclear lysis, islets were sonicated to shear chromatin using a Bioruptor Sonicator (Diagenode) at maximum power; set for 15 seconds ON followed by 45 seconds OFF for a total time of 10 minutes. Precleared chromatin from 200-300 islets was used for each ChIP sample with incubation of 1 to 10 ug of the appropriate antibodies overnight at 4° C. Before IP, 1/10 of the extract was saved for use as the input. Antibodies used for IP were mouse monoclonal anti-RNA Polymerase (1 ug, Millipore, 05-623B), rabbit anti-IgG (1 ug, Santa Cruz, sc-2027), and rabbit anti-NFATc1 (5 ug, Imgenex, #IMG-5101A). Resulting chromatin was then amplified using the GenomePlex WGA4 Whole Genome Amplification Kit (Sigma) and quantified using nanodrop. Equal amounts of chromatin DNA were then analyzed by quantitative PCR in the ABI Prism 7500 detection system (Applied Biosystems) using Power SYBR Green PCR Master Mix (Applied Biosystems). All mouse and human ChIP primer sequences used are listed in Table 2 below, and flank putative NFAT consensus binding sites (T/AGGAAAA/N) within the first 2 kb upstream of the transcriptional start site of each gene.

TABLE 2
PCR Primers used for ChIP analysis on mouse and human gene promoters.

| | | | Targeted NFAT site |
|---|---|---|---|
| Gene Promoter | Forward | Reverse | -BP upstream of start |
| MOUSE | | | |
| CyclinA2 | 5′-GCC TTG CAC TCA AGA GAT CC | 5′-TGA AGT TCC ACT GAC CCA AA | -979 GGAAAA -971 |
| CyclinD2 | 5′-AGA GGG CCT CGG AGA AGT AG | 5′-CAA GCT GGA AGG GCA GTT AG | -65 AGGAAA -58 |
| FoxM1 | 5′-TCA AAG CAG CTC TCC CTT CT | 5′-CGC AGC CTC CTG TGA TAA CT | -793 GGAAA -787 |
| Chromogranin A | 5′-AGT TTC AGC TGT GCC ACC TT | 5′-CAA TGC TAT GCC GGC TTT TA | -311 AGGAAAAC -302 |
| Chromogranin B | 5′-GAG AAA GAG GGG GAG AGG AA | 5′-AAA TCA AAC AGG CCA AAG GA | -329 AGGAAA -322 |
| IA2 | 5′-TCC AAG ACA TCC AGG GCT AC | 5′-TGA CAT TTG GGG TGT GTT TG | -1589 TGGAAATA -1580 |
| Insulin | 5′-AAC TGG TTC ATC AGG CCA TC | 5′-ACT GGG TCC CCA CTA CCT TT | -318 TGGAAAA -310 |
| Glucokinase | 5′-GAA GGA GAA GGG GAA GGA GA | 5′-ATG TTC AGG GCT TGT TCA GG | -1731 GGAAA -1725 |
| HUMAN | | | |
| CYCLINA2 | 5′-AAT TTT TGG CAA GTG GCT GT | 5′-TTT GAA GCC TAT AAA GCG GTC T | -1636 TGGAAAAT -1627 |
| CYCLIND2 | 5′-TTG GCG TGC TAC ACC TAC AG | 5′-CCC CTC CTC CTT TCA ATC TC | -113 GGAAA -107 |
| FOXM1 | 5′-AGG GGC AAA AGA CAG GTT TC | 5′-TCA AAG CTC GGC TTT AGT TGA | -394 AGGAAATC -385 |
| CHROMOGRANIN A | 5′-GTC AGG TGG CAA AGA GCT TC | 5′-CCT TGC AAC ACC TAC CCA TT | -902 AGGAAACT -893 |
| CHROMOGRANIN B | 5′-TGA CTG AAA GAG GAA TTG AGG A | 5′-AAG TGC AGC CGG AGA ATA TG | -523 TGGAAATA -514 |
| IAPP | 5′-GGC GGT TTT GCA GTC ATA TT | 5′-CTA AAA CAG GGC CAA TGG AA | -1701 TGGAAA -1695 |
| IA2 | 5′-TCA TTA TGC ATT TCT GTC CTT TTT | 5′GCT CTT TCA CCA CGA CCA CT | -1212 TGGAAAGC -1203 |
| Negative Sites | | | |
| (Site #2) | | | |
| CYCLINA2 #2 | 5′-GGA GCT ATT CAG CGT GCT TC | 5′-TTC GTG AGT CTG CCC TTC TT | -977 TGGAAAAT -968 |
| CYCLIND2 #2 | 5′-TCA AGC ATG CGT TAG AGC AC | 5′-GGC GAG TGA GGG ATT AGG TC | -187 GGAAA -181 |
| FOXM1 #2 | 5′-TCG TGA CCT CAA GTG ATC CA | 5′-CGC TAG GCC CTG AAG ATA CA | -696 AGGAAAGA -687 |
| CHROMOGRANIN A | 5′-TCT GCC CAA ACT CTG TAC CC | 5′-CTT GAA CCC AAG AGG TGG AG | -1801 TCGAAACC -1792 |
| #2 | | | |
| CHROMOGRANIN B | 5′-GAT TAC AGG CGT GAG CTT CC | 5′-AAG ACC ACA GCC ACA GAA CA | -1328 AGGAAATC -1319 |
| #2 | | | |
| IA2 #2 | 5′-GGA GGG GAG AGA GGA TAT GG | 5′-TCT CGA TCT CCT GAC CTC GT | -1829 GGAAACA -1221 |

Human islet studies. Human islet samples were obtained from healthy, non-diabetic organ donors deceased due to acute traumatic or anoxic death and offered by NDR1 (National Diseases Resource Interchange). Islets were isolated by Bottino, R. at the University of Pittsburg, or by Bryant, S, and Thompson A. at the University of Alabama, Birmingham. Seven independent human islet batches from juvenile donors at the ages of 13 mo (months old), 19 mo, 23 mo, 4 yo (years old), 5 yo, 19 yo and 20 yo, as well as five adult batches from donors of 28, 29, 49, 55, and 56 years of age were used in this study. After isolation, islets were shipped directly to our laboratory and were transferred to fresh islet culture medium (RPMI 1640 with 4.5 mM glucose containing 10% FBS and 1% pen/strep). As with mouse islets, islet samples were split and treated with either vehicle (DMSO) or FK506 (10 mM, LC laboratories) for 72 hours, with medium changes every 24 hours. Islets were then handpicked with dithizone staining and aliquots of 1000 IEQ were spun down and snap frozen for mRNA isolation or crosslinked as described above for ChIP studies. BrdU analysis was performed on the 4 yo donor batch by treating islets with 50 uM BrdU and chasing for 24 hrs. Afterwards, islets were placed in 2% agarose and processed within paraffin blocks as described above. Islets were immunostained for insulin and BrdU and β-cell proliferation rate was determined by quantifying the percentage of insulin+ and BrdU⁺ cells. A minimum of 50 islets and over 2000 β-cells were scored per condition.

In Vitro MIN6 Cell Culture Experiments.

For cell culture experiments, MIN6 murine insulinoma cells (passage 26) were transfected using lipofectamine 2000 (Invitrogen) with 2 ug of either the expression vector alone (pcDNA) or containing human NFATc1 cDNA (courtesy of Dr. Gerald Crabtree) as previously described (Beals et al., 1997). Following transfection, cells were grown for 48 hours and then treated with vehicle (DMSO) or a combination of ionomycin (1 uM) and phorbol 12-myristate 13-acetate (PMA) (25 uM) in order to activate calcineurin/NFAT. Eight hours after induction, cells were harvested for mRNA isolation.

Statistical Analysis.

Results were expressed as the mean±S.E.M. Statistical analysis was performed using the two-tailed or one-tailed, unpaired Student's t-test. Differences were considered to be significant at P<0.05.

### Results

Lethal Postnatal Diabetes from Loss of Pancreatic Islet Cn/NFAT Signaling.

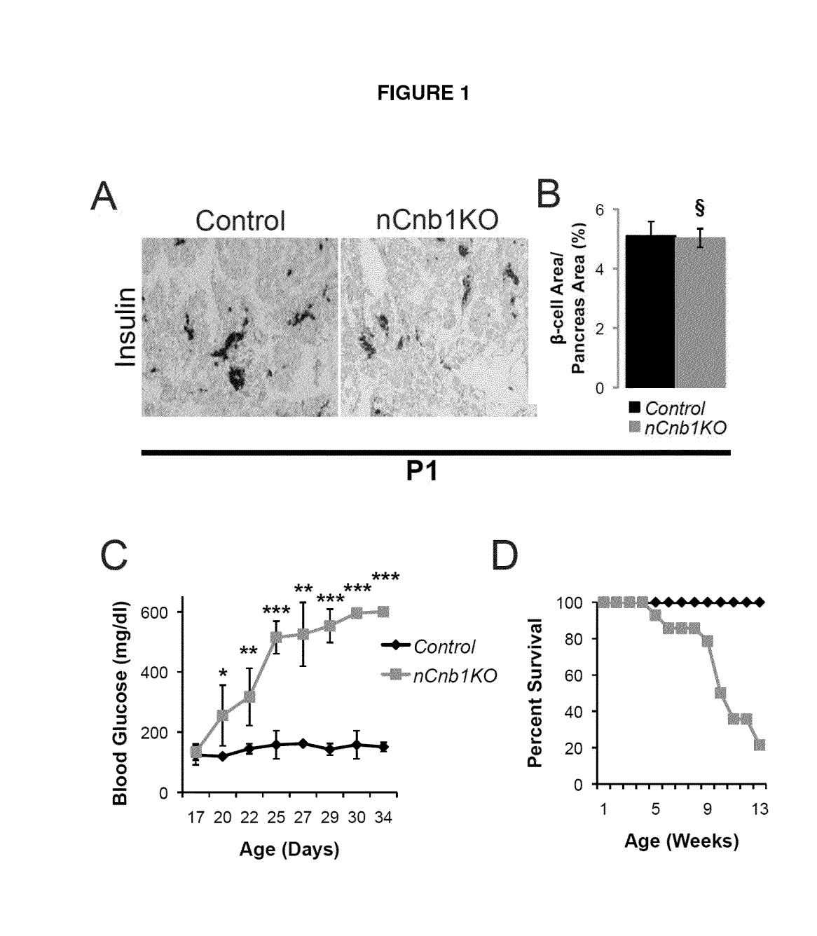

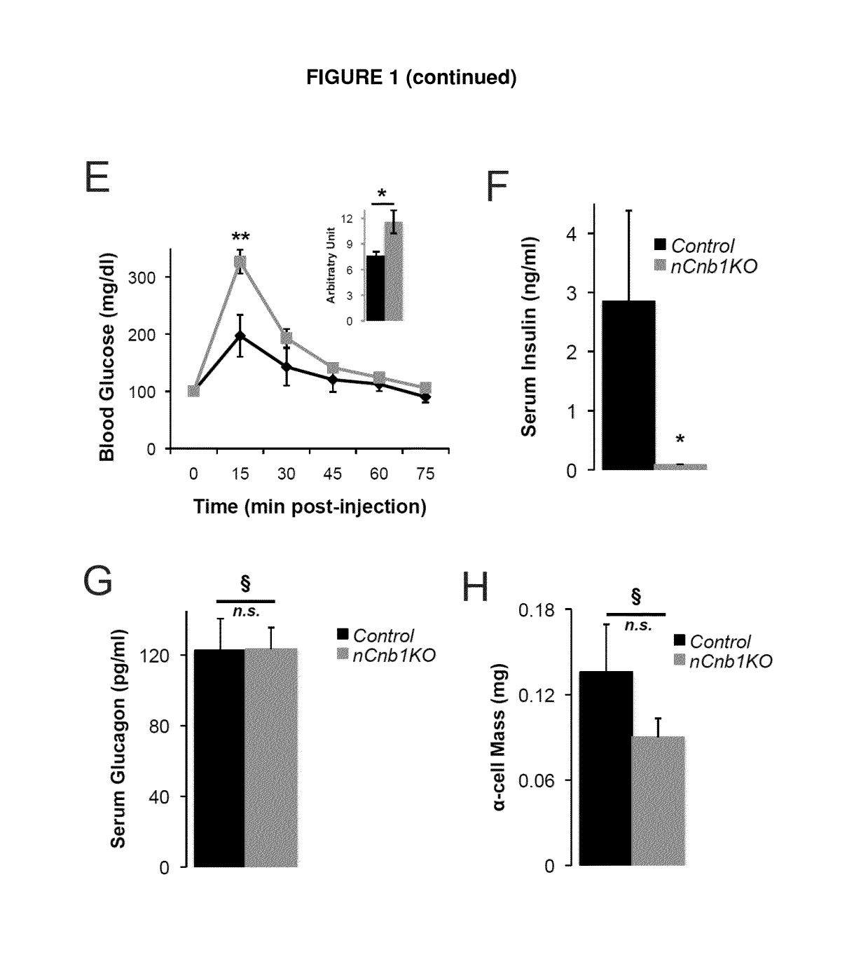

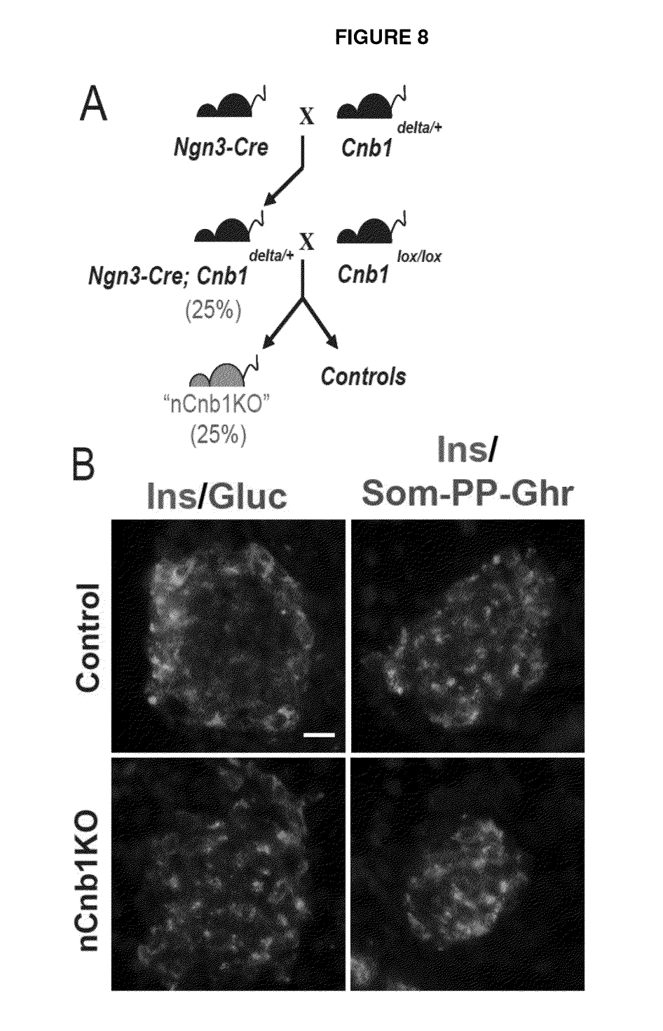

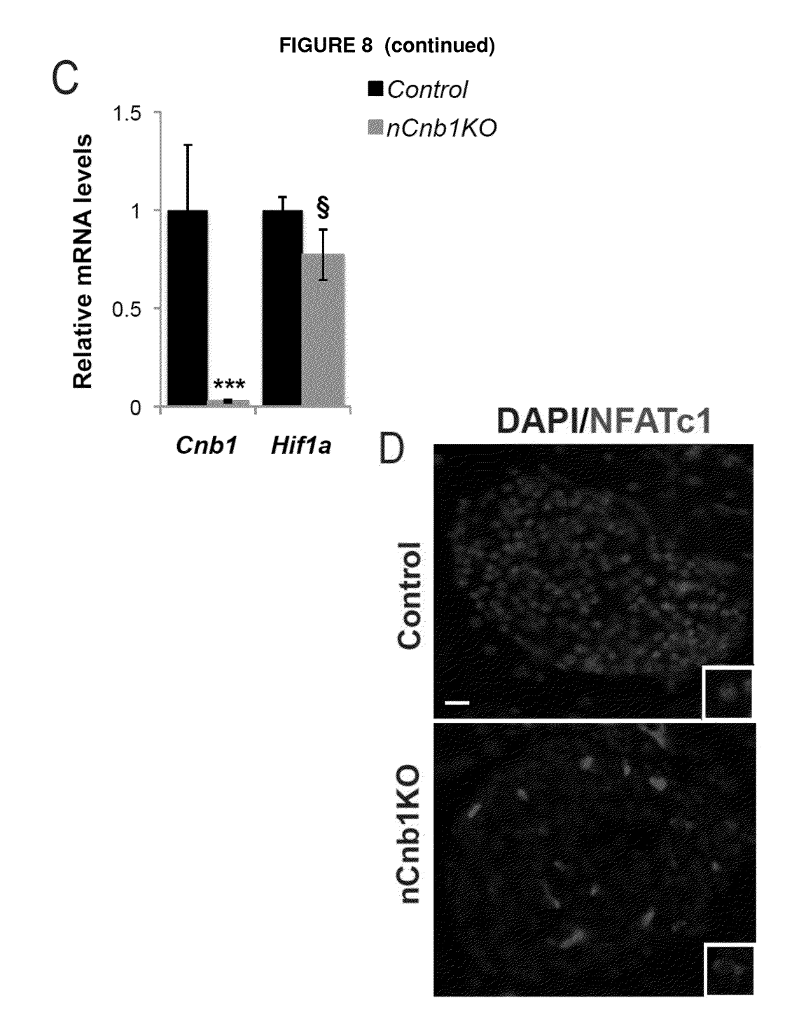

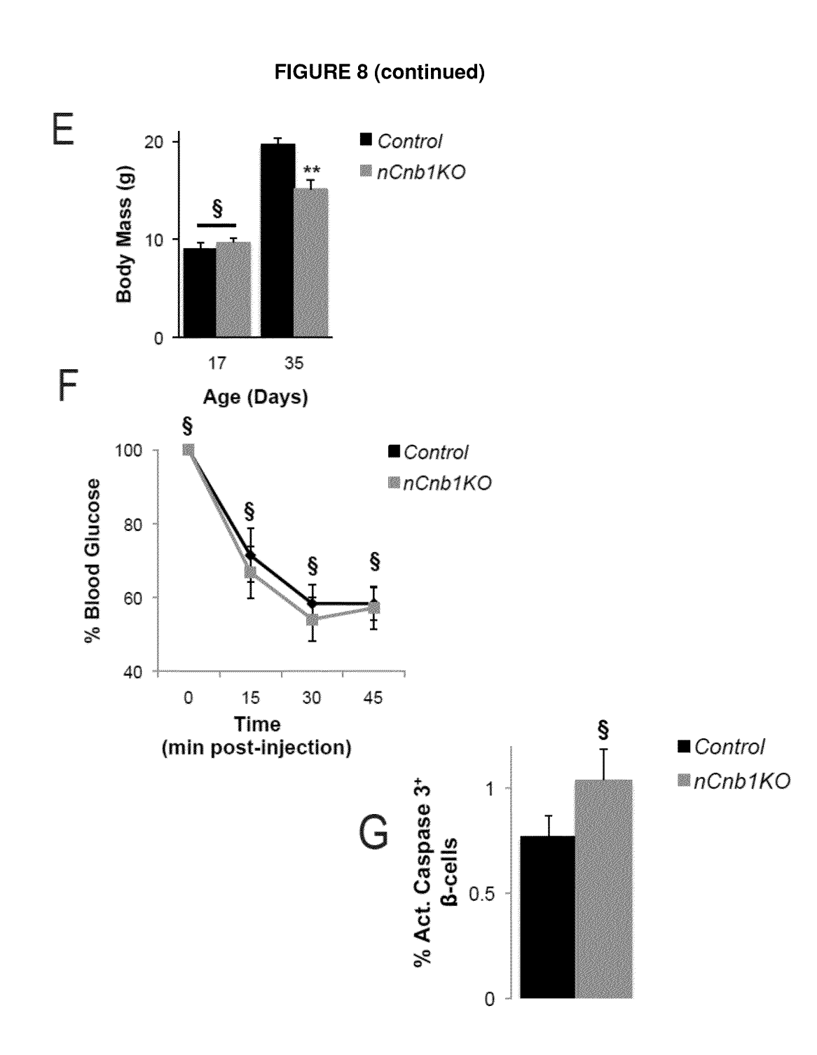

To investigate Cn/NFAT regulation of postnatal β-cell development, we intercrossed mice (FIG. 8A) to produce progeny harboring a Cnb1 null allele)(Cnb1^{Δ}), a Cre recombinase-sensitive conditional allele (Cnb1^{lox}) (Winslow, M. M., et al. (2006). The calcineurin phosphatase complex modulates immunogenic B cell responses. Immunity 24, 141-152) and Ngn3-Cre, which produces Cre in pancreatic endocrine progenitors (Schonhoff, S. E., et al. (2004). Neurogenin 3-expressing progenitor cells in the gastrointestinal tract differentiate into both endocrine and non-endocrine cell types. Dev. Biol 270, 443-454). On postnatal day 1 (P1), Ngn3-Cre; Cnb1^{Δ/lox} mice (hereafter nCnb1KO) had normal β-cell mass (FIG. 1A, B), were born at expected Mendelian frequency (λ²=1.452, P=0.2282) and had normal hormone⁺ islet cell composition (FIG. 8B). Consistent with these findings, serum glucose levels in nCnb1KO mice were indistinguishable from littermate controls before P20 (FIG. 1C). Thus, islet development was not detectably disrupted in embryonic and newborn nCnb1KO mice.

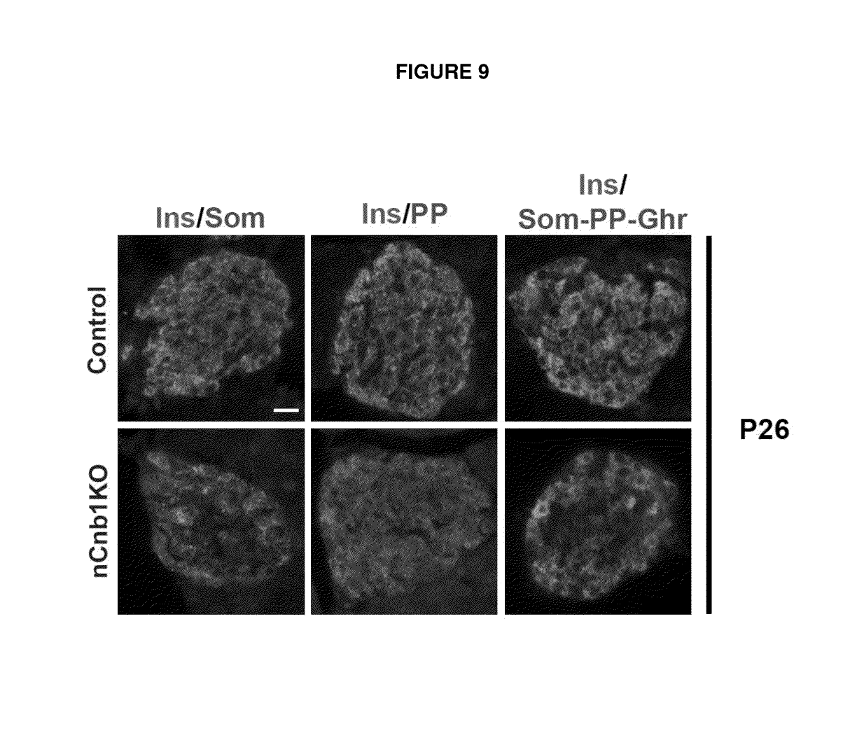

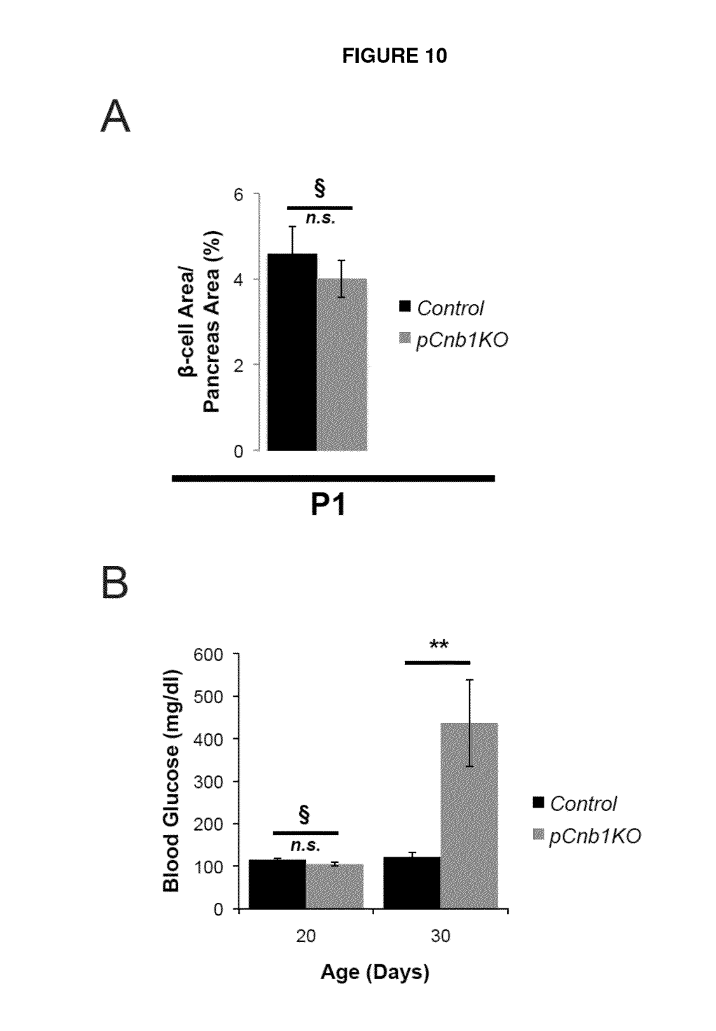

Cnb1 mRNA levels were significantly reduced in postnatal nCnb1KO islets (FIG. 8C) accompanied by reduced nuclear localization and levels of NFATc1 in nCnb1KO β-cells (FIG. 8D), consistent with an established role for NFATc1 in transcriptional auto-regulation of Nfatc1 (Serfling, E., et al. (2006). NFATc1 autoregulation: a crucial step for cell-fate determination. Trends Immunol 27, 461-469). By weaning at P20-21, nCnb1KO mice were overtly diabetic, with severe hyperglycemia (FIG. 1C) and significant weight loss (FIG. 8E). Compared to littermate controls, survival of nCnb1KO mice was compromised, with only 20% viability by 12 weeks of age (FIG. 1D). Thus, Cnb1 inactivation in islet progenitors resulted in a unique model of lethal diabetes in young mice. Insulin challenge studies suggested systemic insulin sensitivity was unaffected in nCnb1KO mice (FIG. 8F). However, intraperitoneal glucose challenge revealed severely impaired glucose tolerance in nCnb1KO mice (FIG. 1E). Serum insulin levels during ad libitum feeding were reduced over 30-fold in nCnb1KO mice by postnatal day 26 (FIG. 1F), suggesting severe β-cell defects. By contrast, neither serum glucagon levels nor α-cell mass were significantly affected in nCnb1KO mice compared to controls (FIG. 1G, H). We did not detect behavioral defects previously described in mice with brain-specific calcineurin inactivation or inhibition (Malleret, G., et al. (2001). Inducible and reversible enhancement of learning, memory, and long-term potentiation by genetic inhibition of calcineurin. Cell 104, 675-686; Miyakawa, T., et al. (2003). Conditional calcineurin knockout mice exhibit multiple abnormal behaviors related to schizophrenia. Proc. Natl. Acad. Sci. U.S.A. 100, 8987-8992; Zeng, H. et al. (2001). Forebrain-specific calcineurin knockout selectively impairs bidirectional synaptic plasticity and working/episodic-like memory. Cell 107, 617-629). Development of other pancreatic islet cells, including delta (somatostatin), pancreatic polypeptide (PP) and epsilon (ghrelin) cells also appeared unchanged in nCnb1KO mice compared to controls (FIG. 8B and FIG. 9). Additionally, Pdx1-Cre; Cnb1^{Δ/lox} (pCnb1KO) mice, in which Cre is expressed from cis-regulatory elements of the pancreatic duodenal homeobox 1 (Pdx1) promoter (Gu, G., et al. (2002). Direct evidence for the pancreatic lineage: NGN3+ cells are islet progenitors and are distinct from duct progenitors. Development 129, 2447-57), phenocopied nCnb1KO mice. pCnb1KO mice had no reduction in β-cell mass at birth but developed lethal diabetes in the early postnatal period (FIG. 10). Together, these findings suggested that disrupted neonatal β-cell development contributed to the lethal diabetes in nCnb1KO mice, and we therefore focused on investigating nCnb1KO β-cell function and growth.

Neonatal β-Cell Development Requires Cnb1.

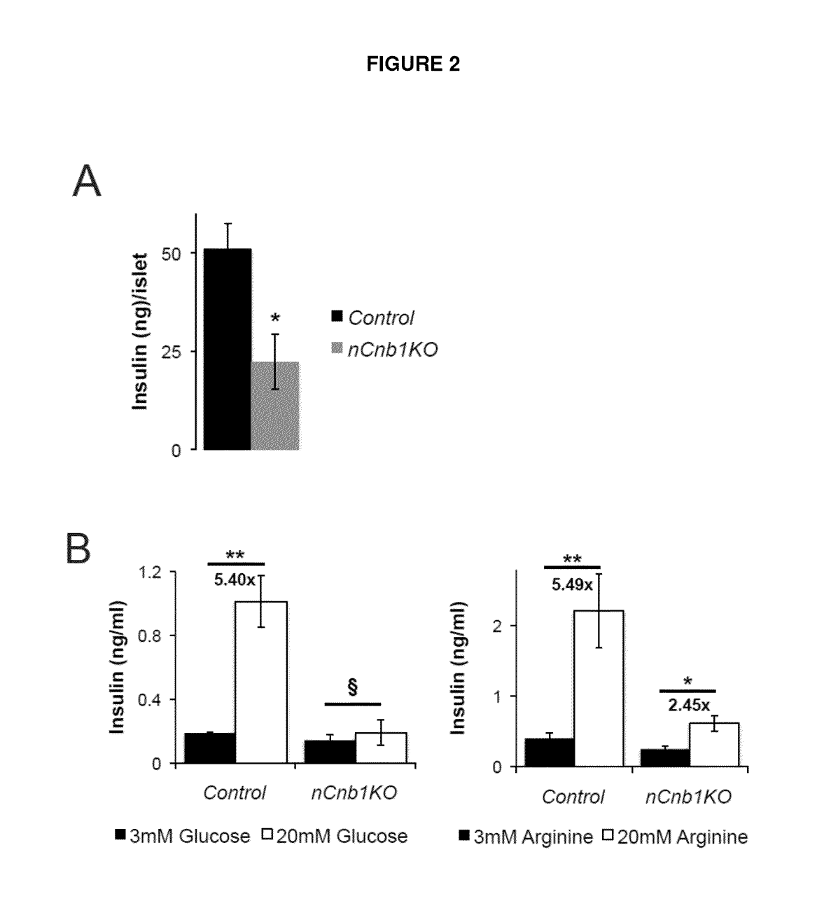

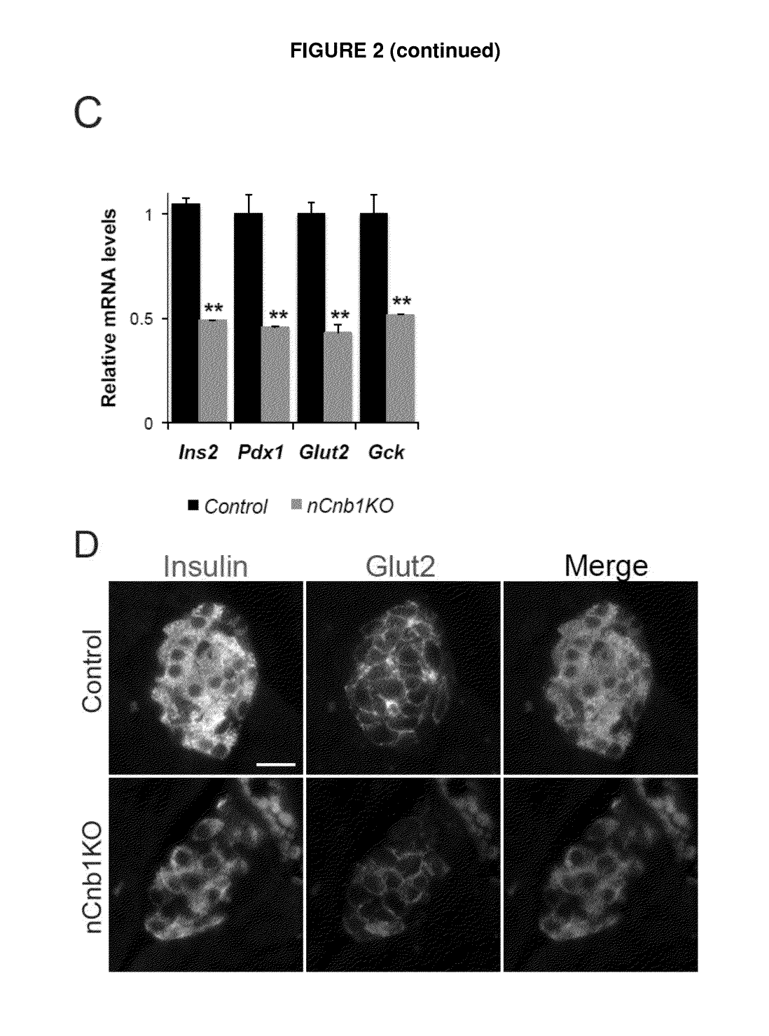

We postulated that β-cell Cnb1 deficiency might compromise β-cell function or growth. Prior to diabetes onset, insulin content was reduced by 50% in nCnb1KO islets compared to size-matched control islets (FIG. 2A). In addition, nCnb1KO β-cells had severely impaired insulin secretion. Cultured islets from prediabetic nCnb1KO P20 mice showed no increase in insulin secretion on glucose challenge (FIG. 2B). Likewise, insulin secretion stimulated by arginine was significantly blunted in nCnb1KO islets (FIG. 2B). Thus, insulin content and secretion were reduced in nCnb1KO islets.

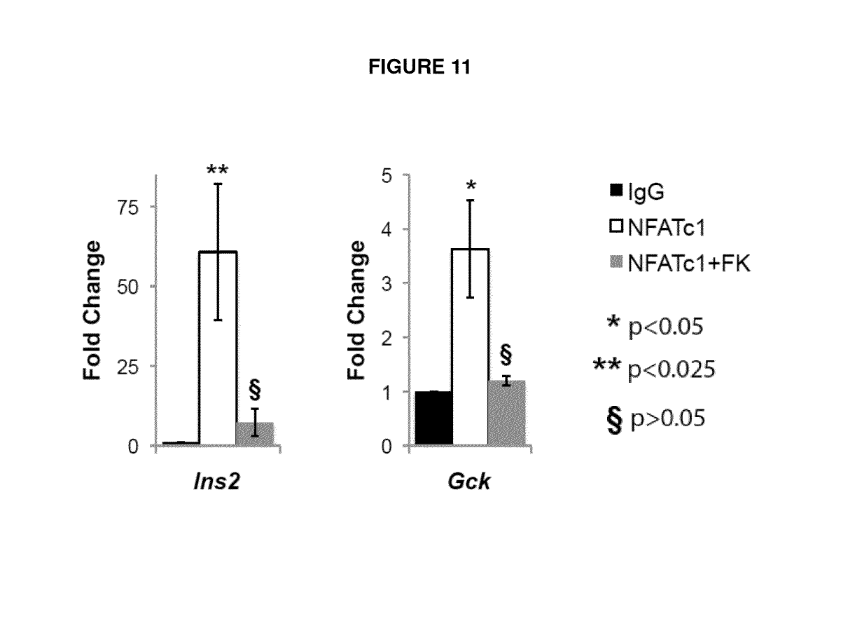

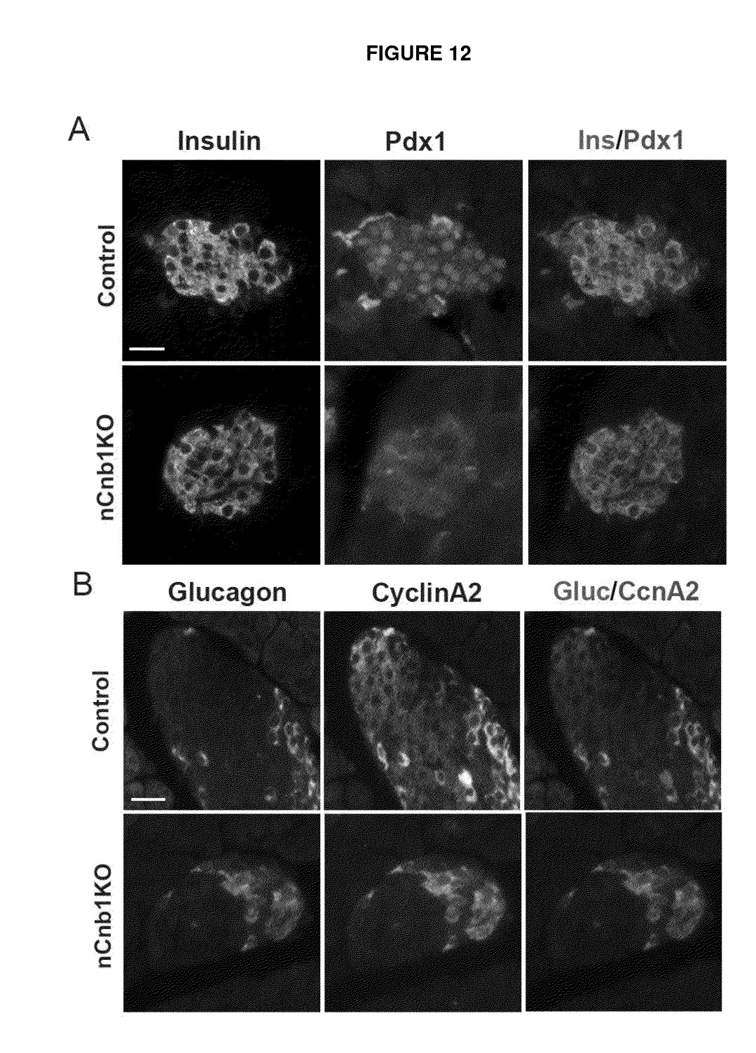

During neonatal β-cell maturation, expression of genes crucial for production and secretion of insulin, including insulin 2 (Ins2), pancreatic duodenal homeobox 1 (Pdx1), type 2 glucose transporter (Glut2), and glucokinase (Gck), increases approximately 10-fold (Aguayo-Mazzucato, C., et al. (2011), supra; Jermendy, A., et al. (2011), supra). Levels of mRNAs encoding all of these factors were reduced in nCnb1KO islets from prediabetic P20 mice (FIG. 2C). Chromatin immunoprecipitation (ChIP) studies of wild type (WT), P20 islets revealed association of NFATc1 at cis-regulatory elements in the Ins2 and Gck gene promoters (FIG. 11), suggesting that NFATc1 directly regulates neonatal islet Ins2 and Gck expression. Consistent with these findings, immunohistology revealed reductions of Insulin, Glut2 and Pdx1 protein in P20 nCnb1KO islets (FIG. 2D and FIG. 12). By contrast, expression of hypoxia inducible factor 1 alpha (Hif1a), another β-cell metabolic regulator (Cheng, K. et al. (2010). Hypoxia-inducible factor-1 alpha regulates beta cell function in mouse and human islets. J. Clin. Invest 120, 2171-2183), was unaltered in nCnb1KO islets (FIG. 8C). Thus, calcineurin signaling is required during β-cell maturation for expression of Insulin, Pdx1, Glut2 and Gck.

Cn/NFAT Signaling Regulates β-Cell Dense Core Granule Formation.

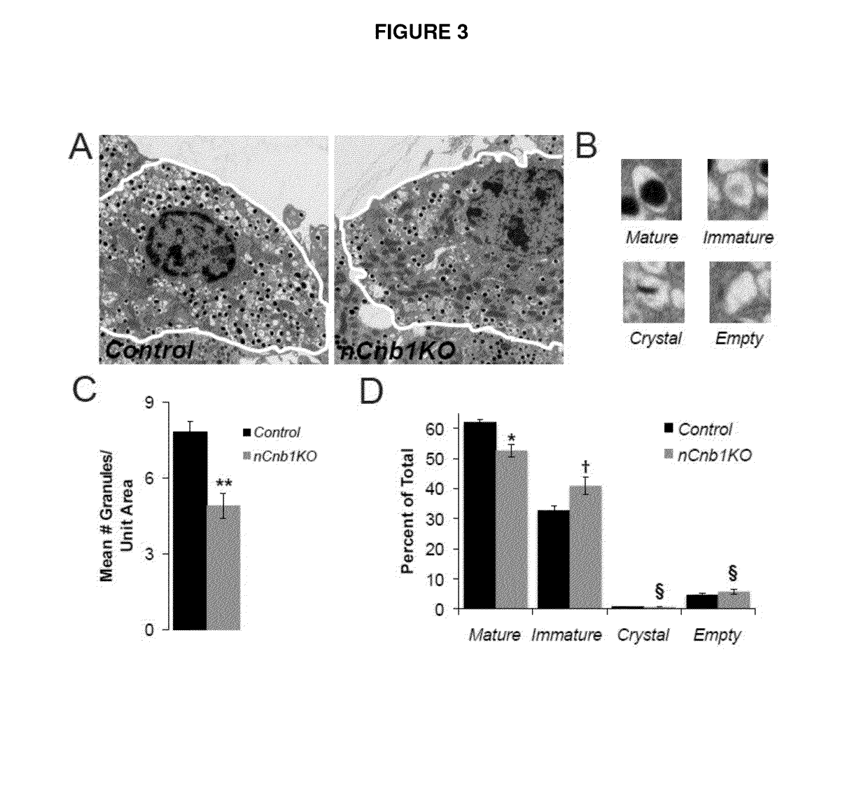

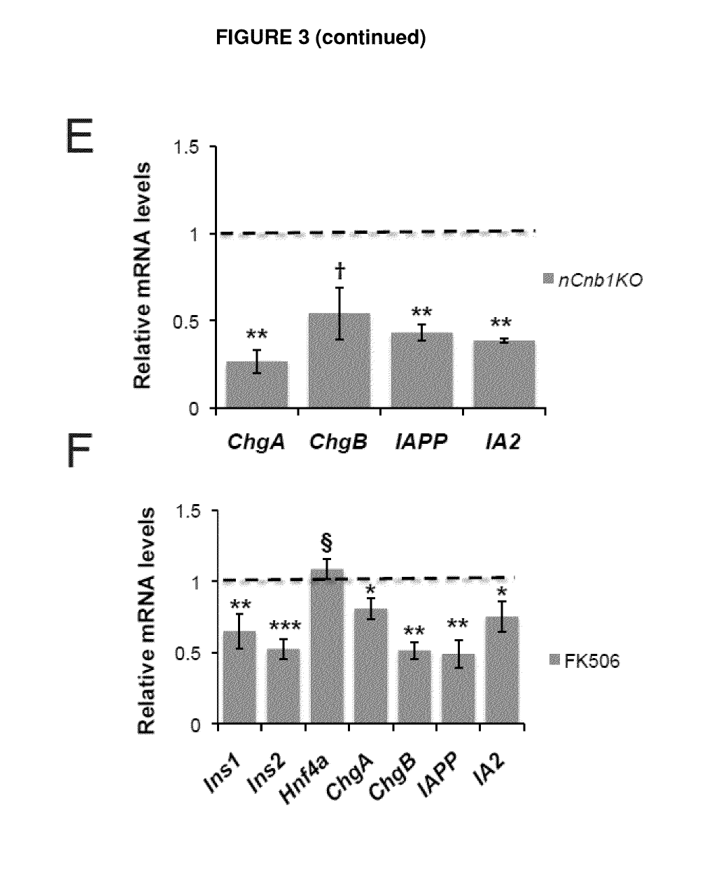

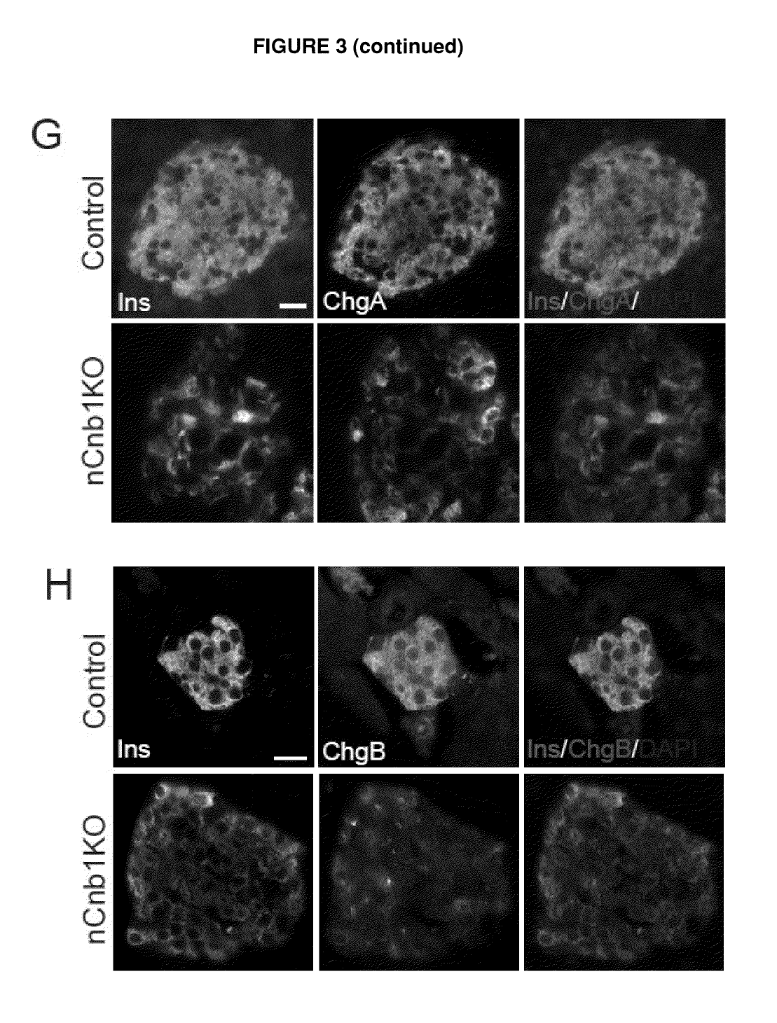

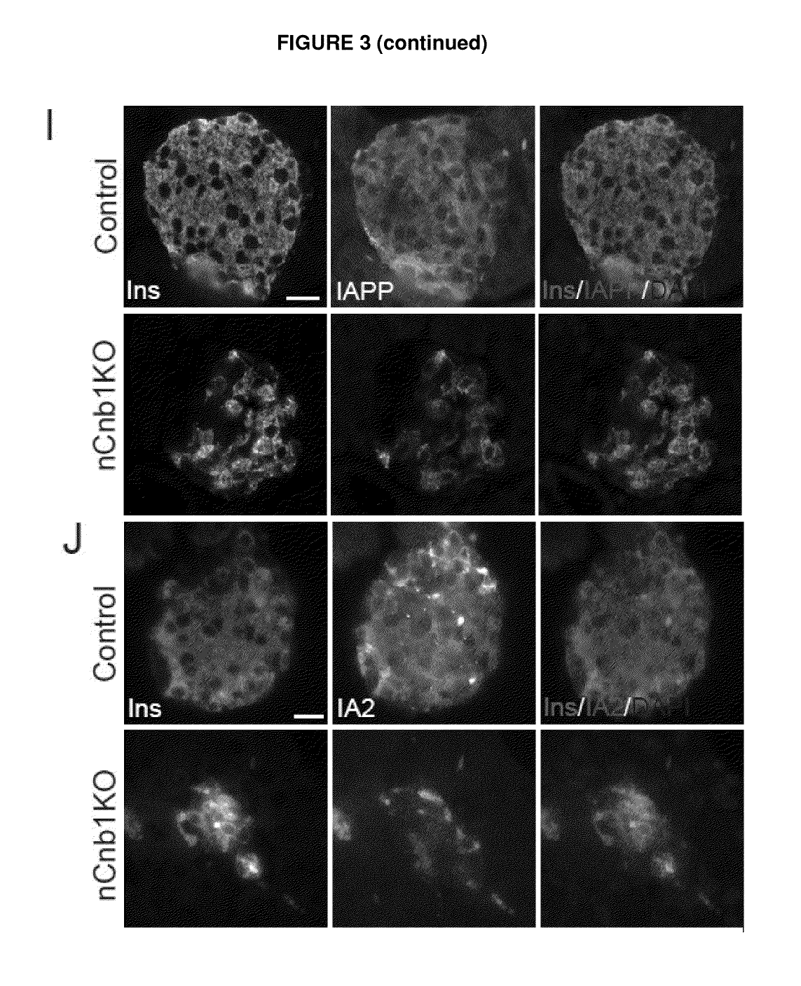

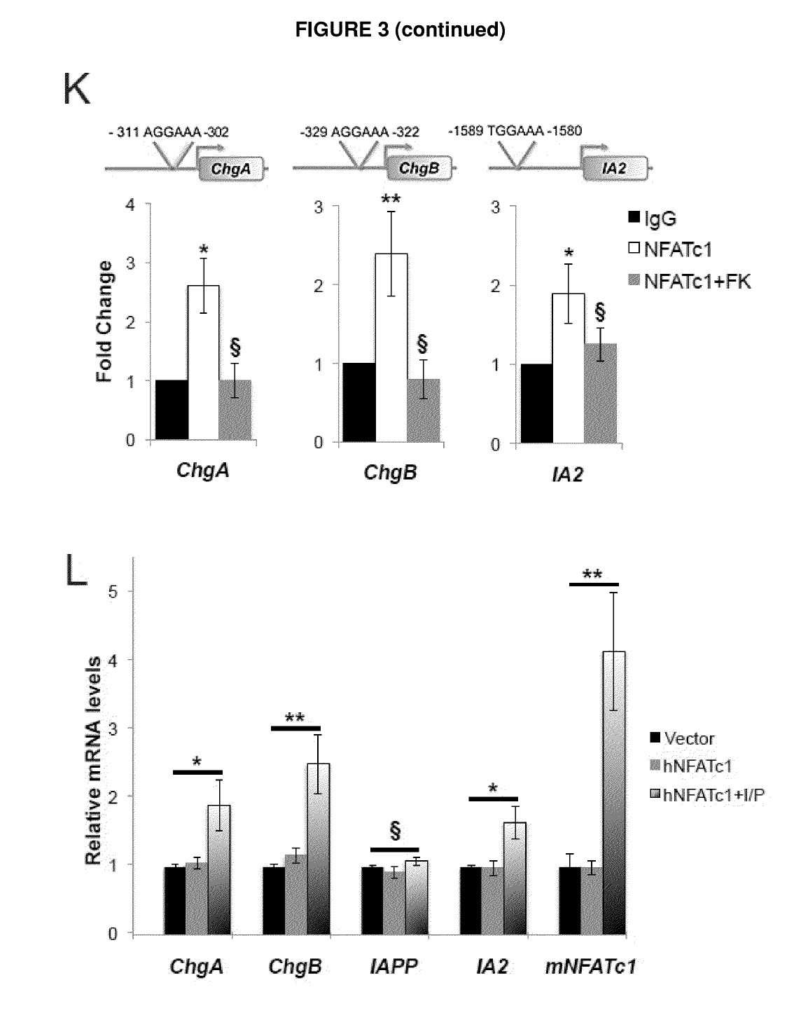

In regulated β-cell secretion, insulin and other proteins are processed and stored in subcellular organelles called dense core granules (DCGs). To determine if Cn/NFAT signaling regulates DCG formation, we investigated DCGs in nCnb1KO mutant and control islets. Based on established morphological criteria (Pictet, R. L., et al. (1972). An ultrastructural analysis of the developing embryonic pancreas. Dev. Biol 29, 436-467), we quantified DCG subsets using transmission electron microscopy, including mature, immature, crystal-containing, and empty DCGs (FIG. 3A, B). nCnb1KO islets from pre-diabetic P20 mice had a 40% decrease in the average number of DCGs in β-cells compared to controls (FIG. 3C). Levels of mature DCGs were also significantly decreased in nCnb1KO β-cells, matched by an increase of immature DCGs (FIG. 3D). The levels of crystal-containing and empty DCGs were not detectably changed in nCnb1KO β-cells (FIG. 3D). These results suggest that Cn/NFAT is required in vivo for the formation and maturation of DCGs.

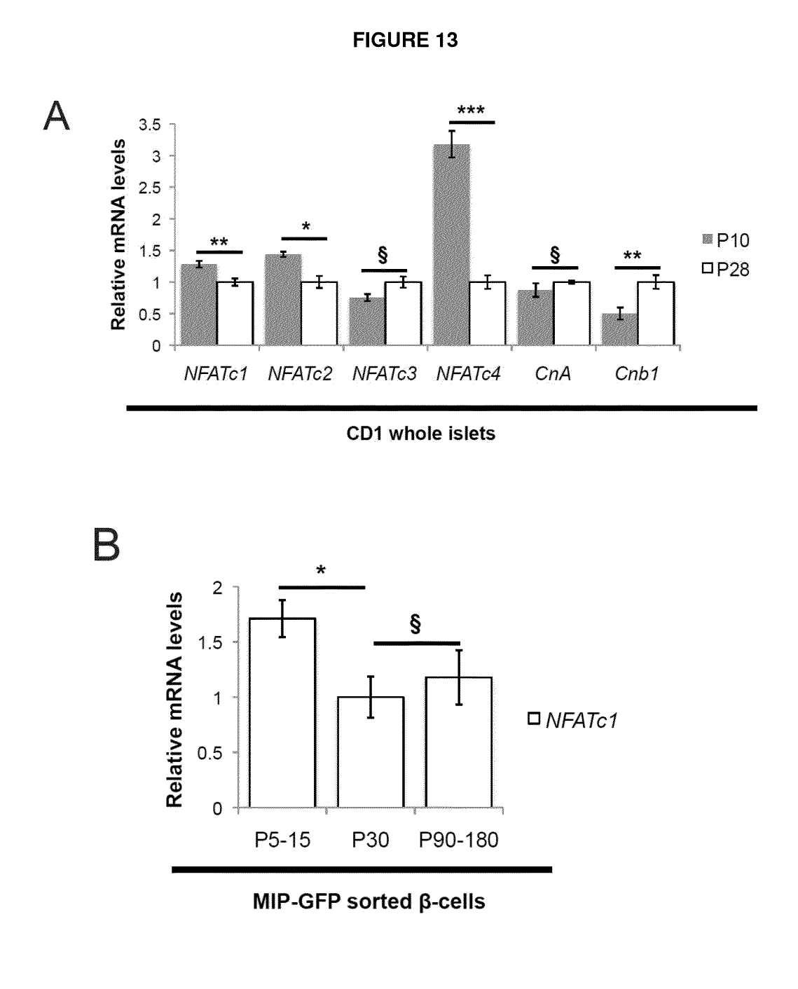

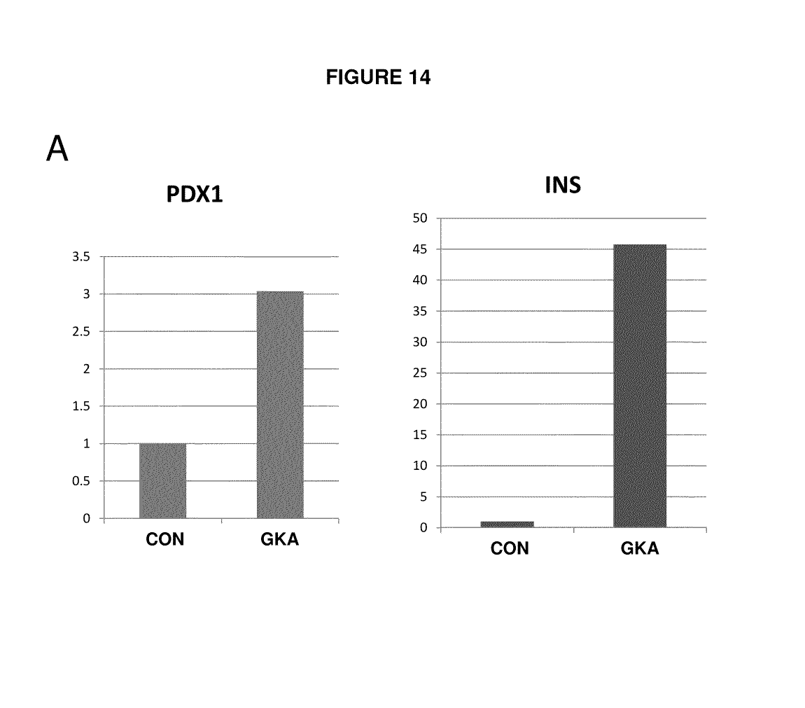

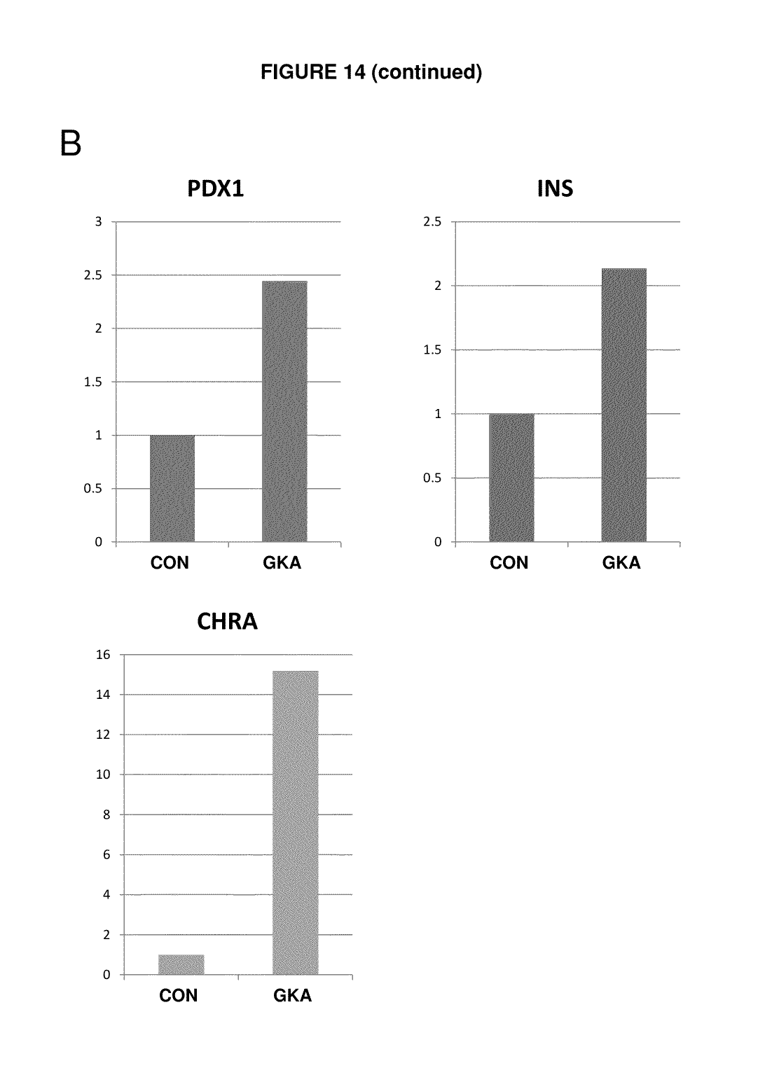

We postulated that impaired expression of genes encoding DCG components might underlie defective DCG formation in nCnb1KO β-cells. Genes encoding DCGs proteins (Hou, J. C., et al. (2009). Insulin granule biogenesis, trafficking and exocytosis. Vitam. Horm 80, 473-506; Kim, T et al. (2006), supra; Suckale and Solimena (2010), supra) include Insulin, chromogranin A (ChgA), chromogranin B (ChgB), islet amyloid polypeptide (IAPP) and the protein tyrosine phosphatase receptor IA2. mRNA and protein levels of ChgA, ChgB, IAPP and IA2 were all decreased in nCnb1KO islets (FIG. 3E, 3G-J). To validate these findings, we exposed islets isolated from WT, P20 mice to the calcineurin inhibitor FK506 (Crabtree and Olson (2002), supra; Lawrence et al. (2001), supra). Compared to control islets exposed to vehicle, islets exposed to FK506 had significantly reduced levels of Ins1, Ins2, ChgA, ChgB, IAPP and IA2 mRNA. By contrast, levels of Hnf4a mRNA, which encodes a regulator of insulin secretion, remained unchanged (FIG. 3F). Thus, genetic and pharmacological loss-of-function studies provide unique evidence that Cnb1 is required for expression of multiple hallmark components of β-cell DCGs. To investigate links between Cnb1 and NFAT in transcriptional regulation of genes encoding DCG components, we used ChIP to test if NFAT associated with cis-regulatory elements in these genes. Using bio-informatic tools, we found consensus NFAT-binding sites in the promoter regions of ChgA, ChgB and IA2, but not IAPP (FIG. 3K; see Materials and Methods). ChIP studies of neonatal, WT islets revealed significant association of NFATc1 at a subset of sites in ChgA, ChgB and IA2 compared to IgG controls (FIG. 3K). In WT islets exposed to FK506, NFATc1 binding at ChgA, ChgB and IA2 promoters was reduced to levels comparable to IgG controls (FIG. 3K). These results suggest that NFATc1 directly regulates Cn-dependent expression of genes encoding hallmark β-cell DCG components. Consistent with a role for NFATc1 in postnatal gene transcription, we discovered that NFATc1 mRNA transcript levels are enriched in β-cells during the postnatal period (FIG. 13).

To determine whether NFATc1 was sufficient to induce levels of DCG components, we expressed human NFATc1 (hNFATc1) (Beals, C. R., et al. (1997). Nuclear localization of NF-ATc by a calcineurin-dependent, cyclosporin-sensitive intramolecular interaction. Genes Dev 11, 824-834) in the murine β-cell line MIN6. Transfected or control cells were then exposed to ionomycin and phorbol 12-myristate 13-acetate (PMA), factors that stimulate NFAT nuclear localization and activity (Beals, C. R., et al. (1997). Nuclear localization of NF-ATc by a calcineurin-dependent, cyclosporin-sensitive intramolecular interaction. Genes Dev 11, 824-834), or to vehicle. Consistent with prior work revealing NFATc1 transcriptional auto-regulation (Serfling, E., et al. (2006). NFATc1 autoregulation: a crucial step for cell-fate determination. Trends Immunol 27, 461-469), we observed that levels of murine NFATc1 (mNFATc1) mRNA increased in MIN6 cells transfected with hNFAT and induced with ionomycin/PMA (FIG. 3L). By contrast mNFATc1 levels were not elevated in MIN6 cells expressing hNFATc1 exposed to vehicle, as anticipated (FIG. 3L). Levels of mRNAs encoding the β-cell NFAT targets ChgA, ChgB and IA2 were also increased in hNFATc-transfected MIN6 cells exposed to ionomycin/PMA (FIG. 3L). By contrast, IAPP mRNA levels were not increased, consistent with our ChIP results (FIG. 3K). Thus, Cn/NFAT signaling is sufficient to stimulate expression of genes encoding mouse β-cell DCG components.

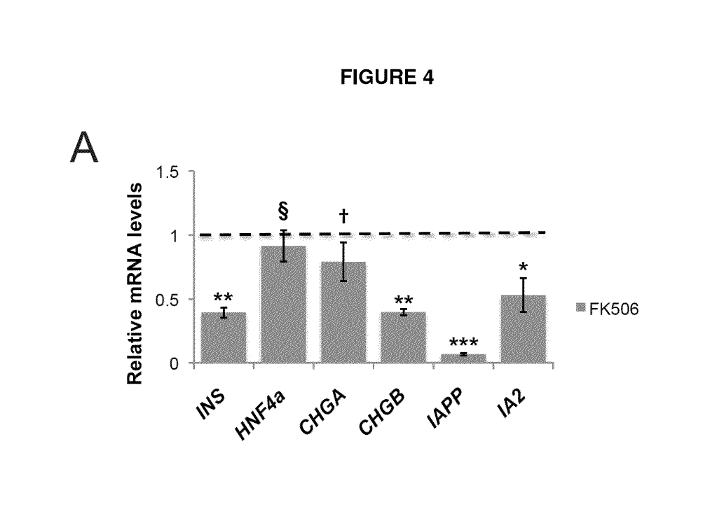

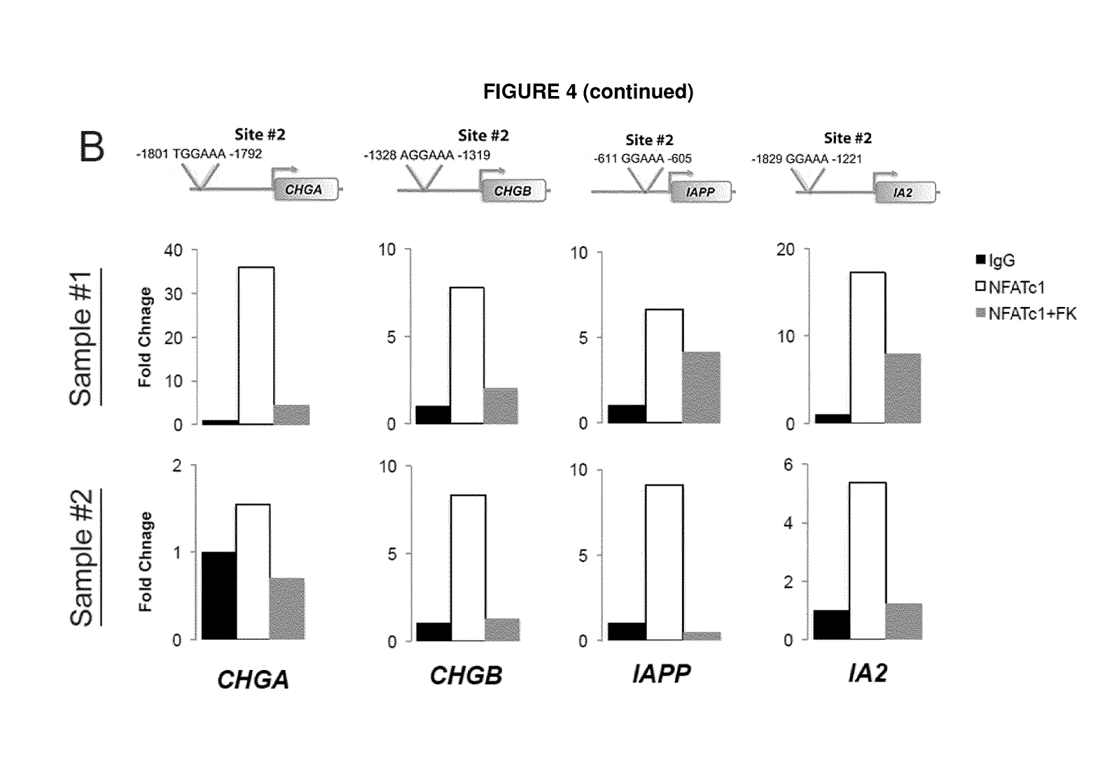

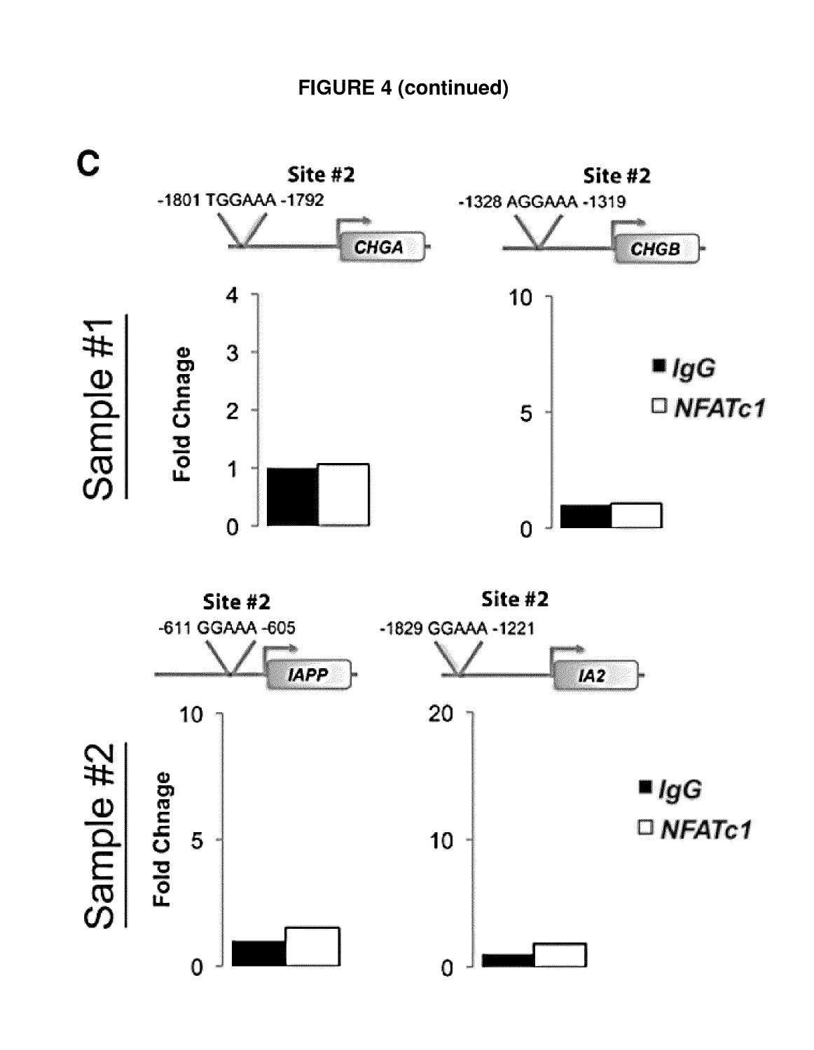

To investigate if Cn/NFAT regulation of DCG components was conserved, we studied cultured human islets exposed to FK506 or vehicle control. Compared to control islets, we found reduction of mRNAs encoding INS, CHGA, CHGB, IAPP, and IA2 in islets exposed to FK506 (FIG. 4A). By contrast, islet levels of mRNA encoding HNF4a were unaffected by FK506 (FIG. 4A). Thus, similar to findings from mouse islets (FIGS. 3E and 3F), expression of genes encoding the principal DCG components in human islets was reduced by calcineurin inhibition. We next used ChIP to investigate if NFAT associated with cis-regulatory elements in CHGA, CHGB, IAPP, and IA2. Based on discovery of candidate NFAT-binding sites within the promoter regions of CHGA, CHGB, IAPP, and IA2 (FIG. 4B; see Experimental Procedures), ChIP revealed significant association of NFATc1 at a subset of sites in all these loci, compared to IgG controls (FIGS. 4B and 4C). In human islets exposed to FK506, NFATc1 binding at these targets was consistently reduced (FIG. 4B). Thus, Cn/NFAT signaling regulates expression of hallmark β cell DCG components in human islets.

Proliferation to Establish Adequate Neonatal β-Cell Mass Requires Cn/NFAT Signaling.

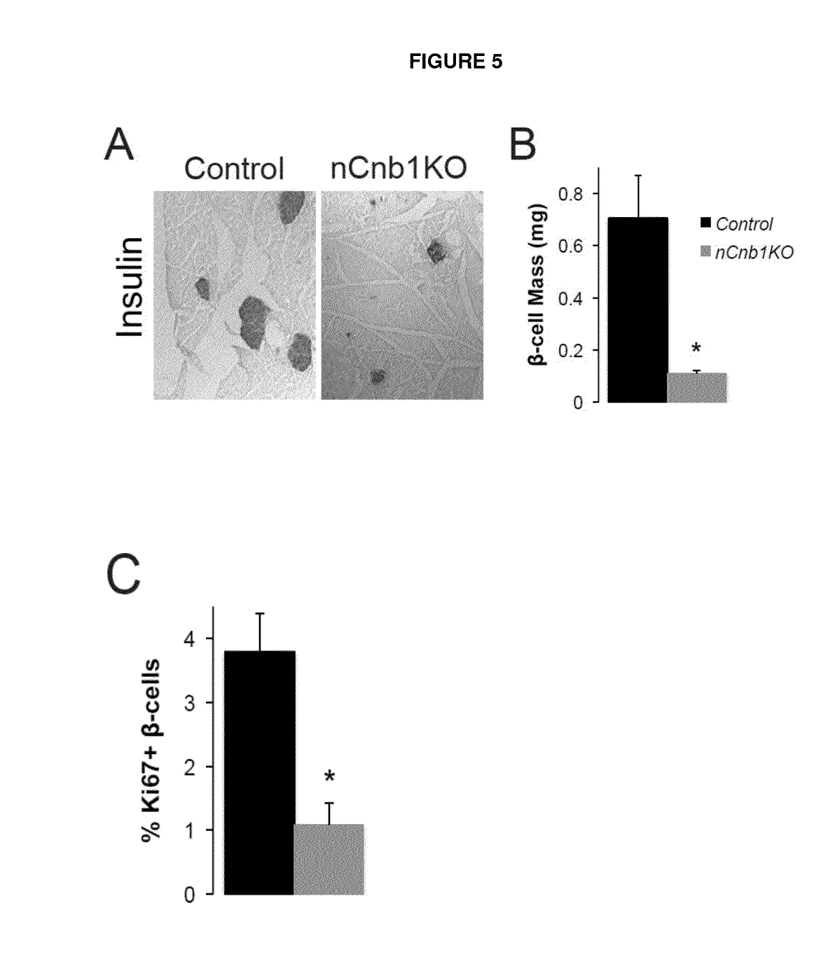

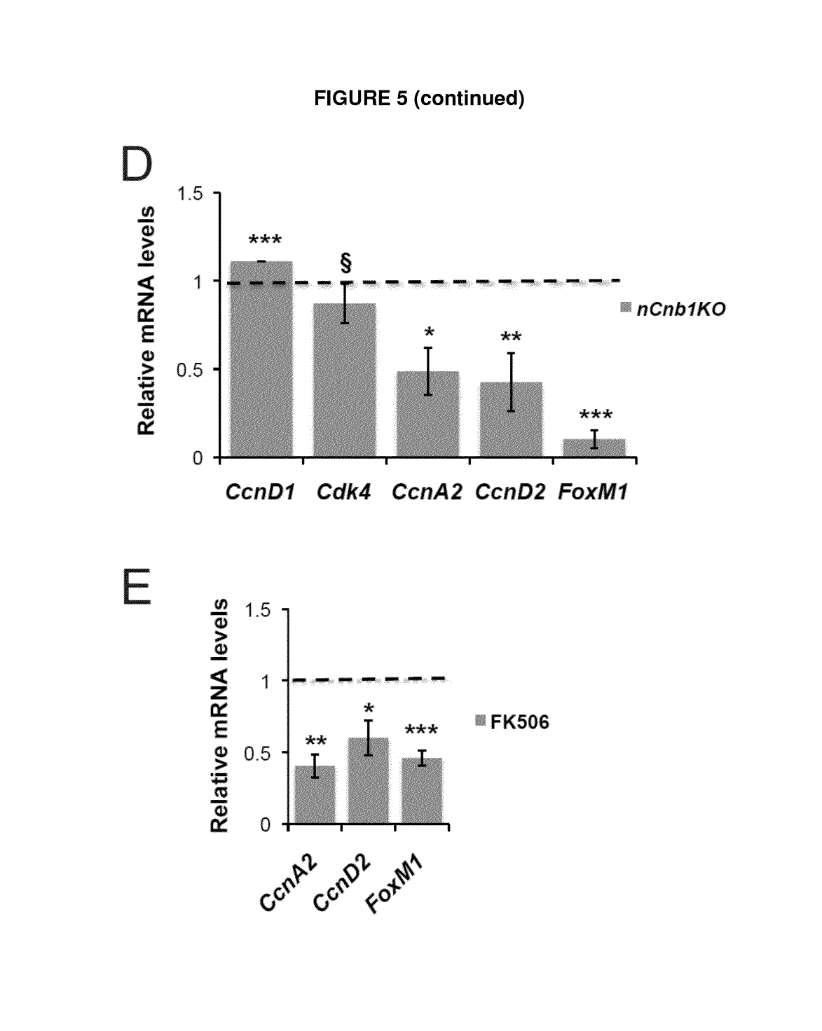

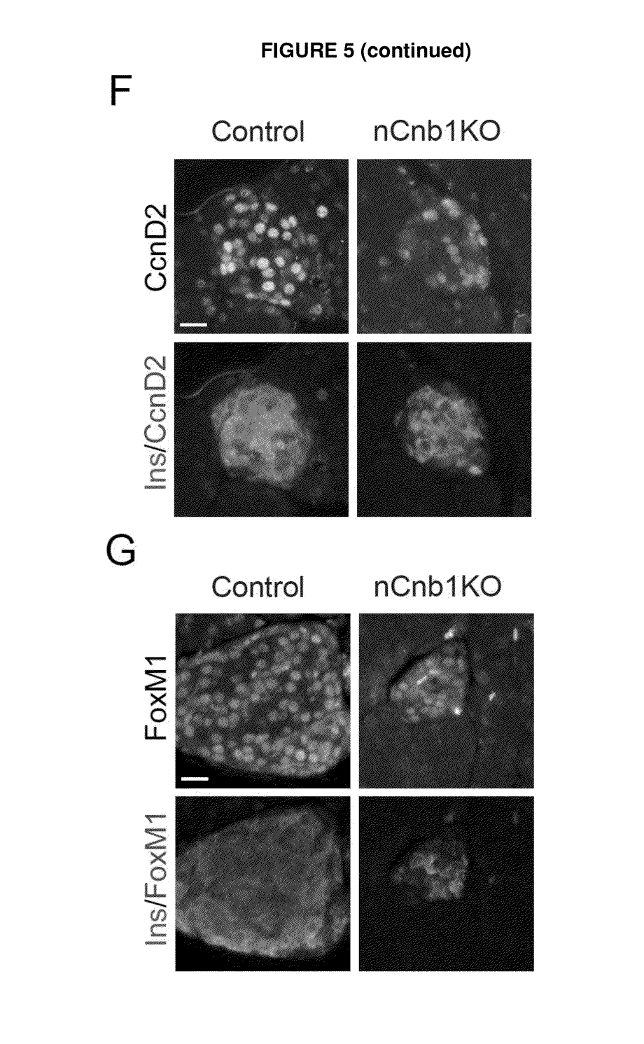

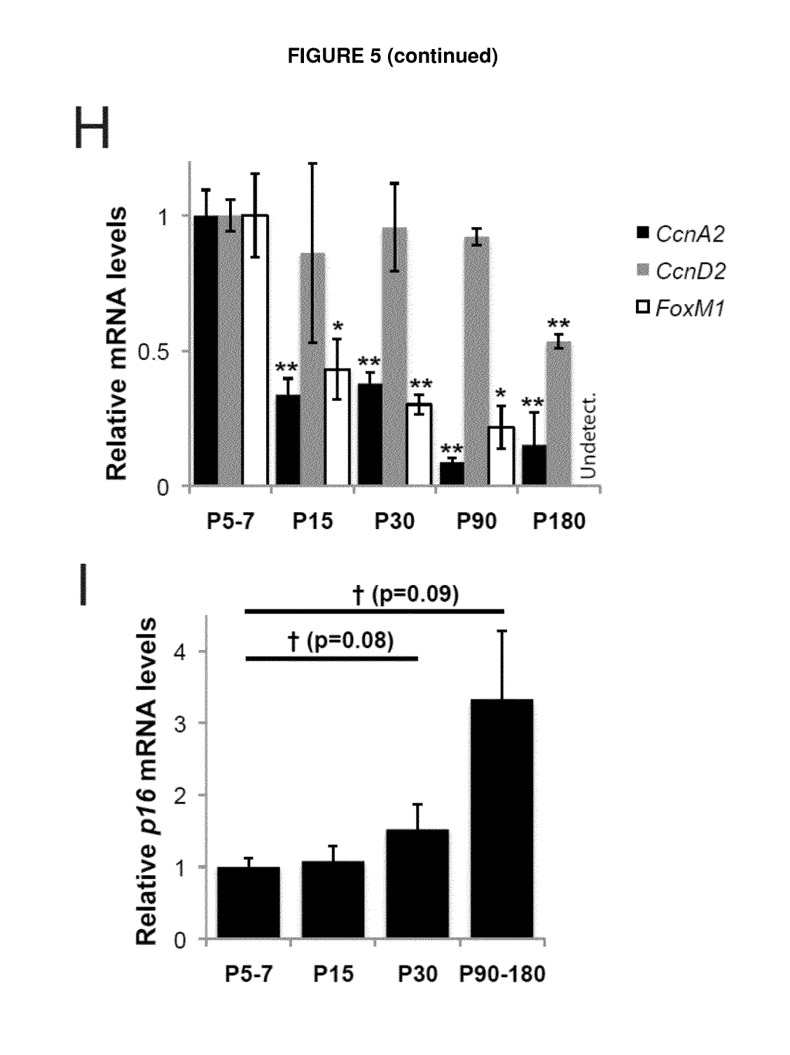

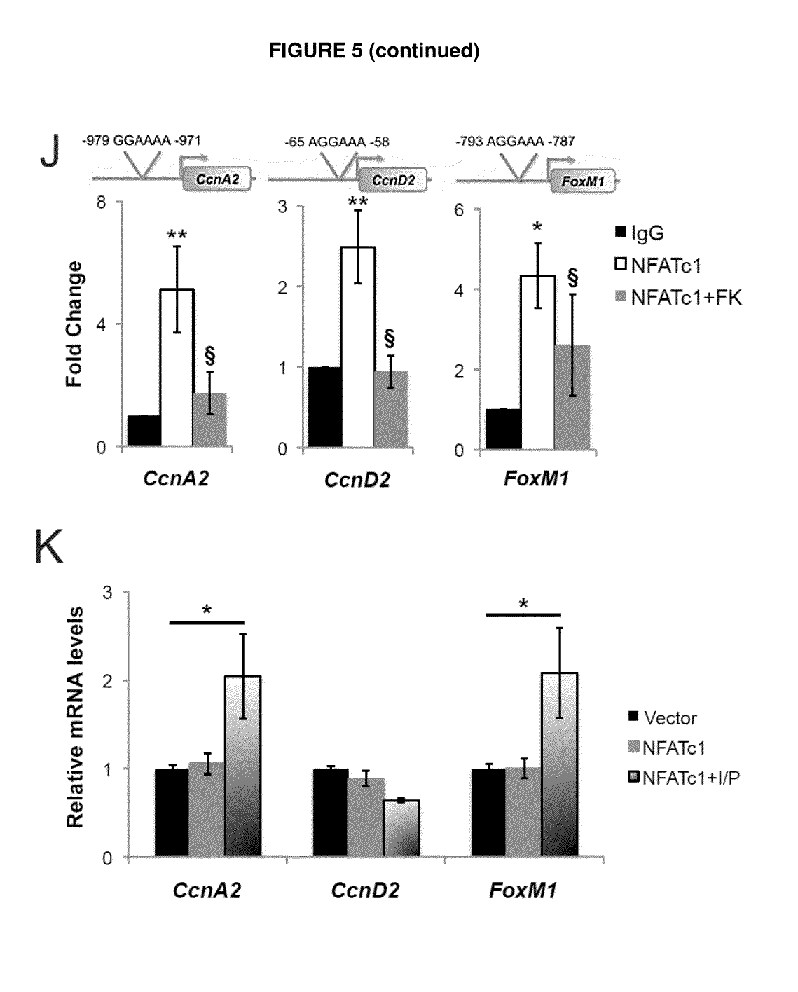

To investigate whether Cn/NFAT signaling regulates postnatal β-cell proliferation, we examined nCnb1KO pancreata. Compared to littermate controls, nCnb1KO mice at P26 exhibited a 7-fold decrease in β-cell mass (FIG. 5A, B). In nCnb1KO mice at P26 we observed a 3-fold reduction in β-cells expressing the proliferation marker Ki67, indicating impaired β-cell proliferation (FIG. 5C). By contrast, the percentage of β-cells immunostained for activated caspase 3, a marker of apoptosis, was not significantly increased (FIG. 8G). Thus, Cnb1 is required for neonatal β-cell proliferation and expansion. To identify the basis for impaired expansion of juvenile β-cells lacking Cnb1, we measured expression of known regulators of neonatal β-cell proliferation, including cyclin D1 (Ccnd1), cyclin D2 (Ccnd2), cyclin-dependent kinase 4 (Cdk4), and the Forkhead box (Fox) factor FoxM1 (Kushner et al. (2005), supra; Rane et al. (1999) supra; Zhang et al. (2006), supra). In islets from pre-diabetic P20 nCnb1KO mice, CcnD2 and FoxM1 mRNAs were significantly reduced compared to levels in size- and age-matched control islets. Likewise, nCnb1KO islet mRNA levels of Cyclin A2 (CcnA2), another regulator of the G1-to-S phase transition, were also reduced (FIG. 5D). By contrast, CcnD1 and Cdk4 mRNAs were not detectably reduced in nCnb1KO islets (FIG. 5D). To validate these findings, we exposed neonatal, WT islets to FK506. Like in nCnb1KO islets, levels of CcnA2, CcnD2 and FoxM1 mRNAs were significantly decreased (FIG. 5E). Immunohistology revealed accompanying reductions of CcnD2 and FoxM1 protein in nCnb1KO β-cell nuclei (FIG. 5F-G). These results reveal a requirement for Cn/NFAT signaling during a crucial, distinct neonatal stage of β-cell proliferation and expansion.

CcnA2, CcnD2 and FoxM1 Regulation in Mouse and Human Islets by NFATc1.

To investigate CcnD2, FoxM1 and CcnA2 regulation in islets, we measured mRNA levels encoding these factors in β-cells purified by FACS from juvenile and adult MIP-GFP mouse islets (Sugiyama, T., et al. (2007). Conserved markers of fetal pancreatic epithelium permit prospective isolation of islet progenitor cells by FACS. Proc. Natl. Acad. Sci. U.S.A 104, 175-180). Levels of mRNAs encoding CcnD2, FoxM1 (Ackermann Misfeldt A, et al. (2008). Beta-cell proliferation, but not neogenesis, following 60% partial pancreatectomy is impaired in the absence of FoxM1. Diabetes 57, 3069-77) and CcnA2 were enriched in juvenile β-cells, then declined in adults (FIG. 5H). By contrast, mRNA levels encoding the cyclin-dependent kinase inhibitor p16^{Ink4a} increased during this interval (FIG. 51), confirming prior results (Chen, H., et al. (2009). Polycomb protein Ezh2 regulates pancreatic beta-cell Ink4a/Arf expression and regeneration in diabetes mellitus. Genes Dev 23, 975-985). Age-dependent β-cell expression of CcnD2 and FoxM1 revealed here agrees with prior reports that CyclinD2 and FoxM1 protein abundance is greatest in neonatal mouse β-cells (Georgia and Bhushan (2004), supra; Zhang et al. (2006), supra). Consistent with the view that Cn/NFAT regulates islet CcnD2, FoxM1 and CcnA2 expression, we identified promoterproximal consensus NFAT-binding sites in these loci (FIG. 5J). NFATc1 protein association at these sites in WT, neonatal islets was revealed by ChIP and exposure of islets to FK506 consistently reduced NFATc1 binding to background levels (FIG. 5J). Thus, NFATc1 directs in vivo expression of established regulators of neonatal β-cell proliferation.

To test whether NFATc1 was sufficient to induce the expression of these cell cycle regulators, we expressed human NFATc1 (hNFATc1) (Beals, C. R., et al. (1997). Nuclear localization of NF-ATc by a calcineurin-dependent, cyclosporin-sensitive intramolecular interaction. Genes Dev 11, 824-834) in the MIN6 β-cell line. hNFATc1-transfected cells were then exposed to ionomycin/PMA or to vehicle. hNFATc1 transfection followed by ionomycin/PMA treatment increased expression of CcnA2 and FoxM1, compared to controls (FIG. 5K). Baseline levels of CcnD2 in MIN6 cells are known to be elevated (Cozar-Castellano, 1., et al. (2006). Molecular control of cell cycle progression in the pancreatic beta-cell. Endocr. Rev 27, 356-370); thus, as expected, hNFATc1 transfection with ionomycin/PMA treatment did not further increase CcnD2 mRNA in MIN6 cells (FIG. 5K). Collectively, these data demonstrate a novel role for Cn/NFAT in regulating CcnA2, CcnD2 and FoxM1 expression and β-cell proliferation in postnatal mouse islets.

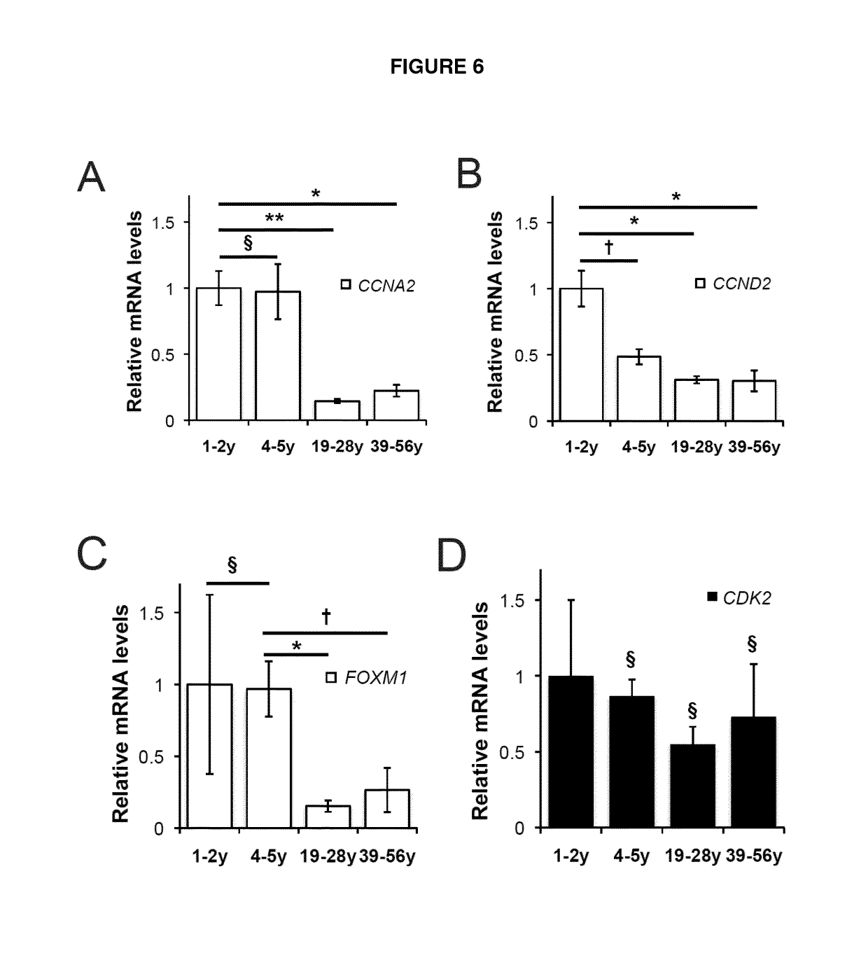

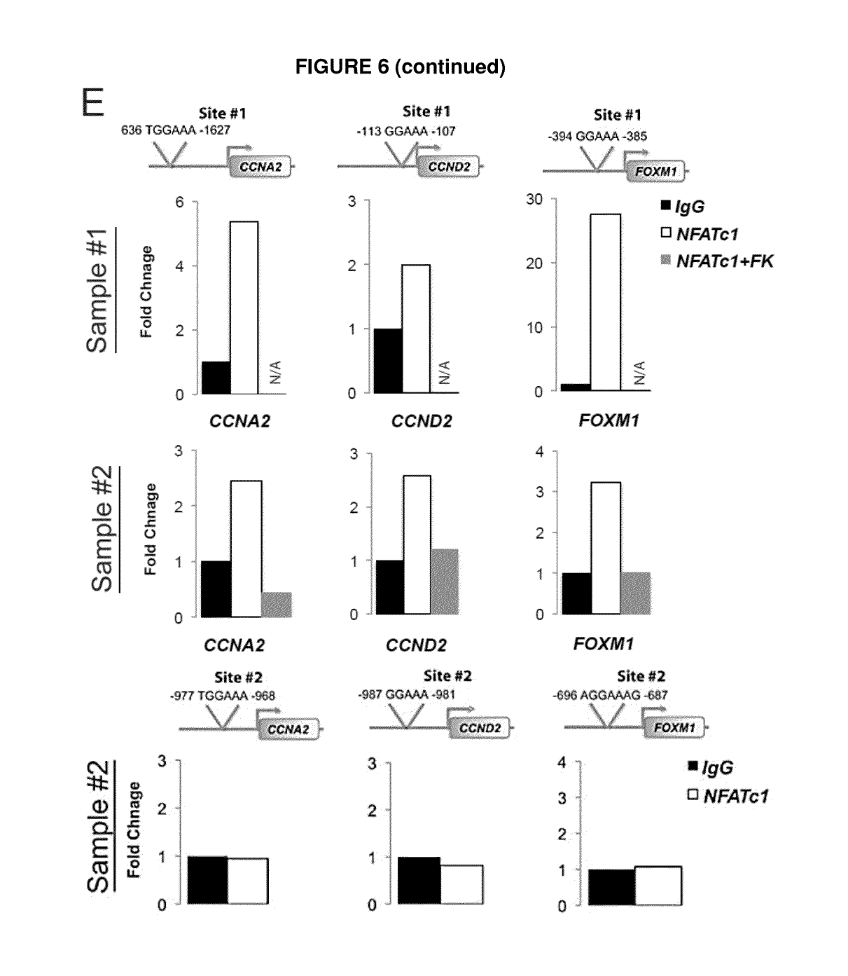

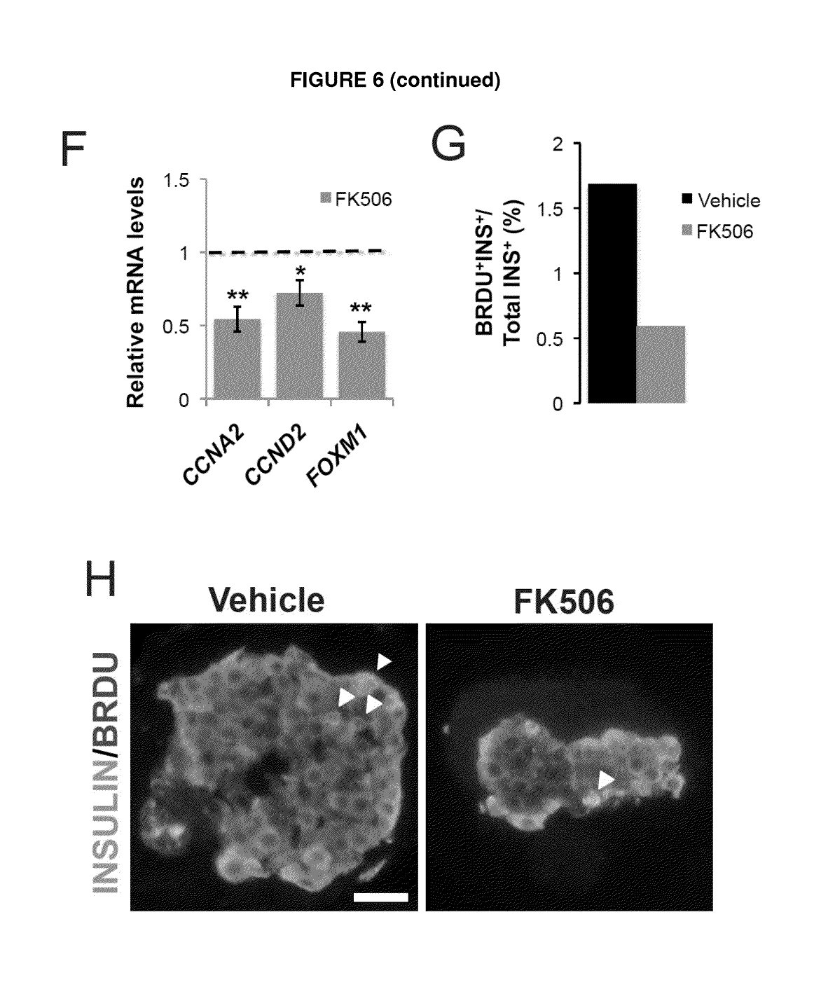

Human β-cell proliferation, assessed by Ki67 staining, is highest in children less than 5-10 years of age (Meier, J. J et al. (2008). Beta-cell replication is the primary mechanism subserving the postnatal expansion of beta-cell mass in humans. Diabetes 57, 1584-1594). To test the relevance of our findings to human β-cell proliferation, we measured mRNA levels of CCNA2, CCND2 and FOXM1 in islets isolated from donors aged 1-5 years and control adult donors. mRNAs encoding CCNA2, CCND2 and FOXM1 were higher in islets purified from young donors than in islets from adults (FIG. 6A-C). By contrast, levels of mRNA encoding cyclin-dependent kinase 2 (CDK2) did not change significantly with human islet age (FIG. 6D). Thus, our findings reveal elevated islet expression of CCNA2, CCND2 and FOXM1 during a period of established, physiologic human β-cell expansion. Based on similarities to our findings with age-dependent expression of these factors in mice, we tested the possibility that Cn/NFAT signaling governs human islet CCNA2, CCND2 and FOXM1 expression. We identified consensus NFAT-binding sites in the promoter regions of human CCNA2, CCND2 and FOXM1 (FIG. 6E), and ChIP revealed FK506-sensitive NFATc1 association at these sites in neonatal human islets (FIG. 6E). Next, we exposed human islets to FK506 or vehicle control, and QPCR revealed that CCNA2, CCND2 and FOXM1 mRNA levels were significantly decreased in FK506-exposed islets compared to control islets (FIG. 6F). Consistent with these findings, exposure of cultured neonatal human islets to FK506 reduced β-cell BrdU incorporation by nearly 3-fold, compared to islets exposed to vehicle control (FIG. 6G-H). Collectively, these findings provide unique evidence that Cn/NFAT signaling regulates CCNA2, CCND2 and FOXM1 expression and β-cell proliferation in human islets.

Glucokinase Activator Induces NFAT Target Genes Governing β-Cell Growth and Maturation.

Our findings suggested that Cn/NFAT signaling is developmentally regulated in postnatal islets, with elevated signaling in neonatal islets followed by reduction in adult islets. If so, we predicted that expression of mRNAs encoding NFATc1 and other components of this pathway would be elevated in neonatal islets compared to those in adult islets, since NFATc1 activates expression of itself and other pathway components (Arron, J. R., et al. (2006). NFAT dysregulation by increased dosage of DSCR1 and DYRK1a on chromosome 21. Nature 441, 595-600; Serfling, E., et al. (2006). NFATc1 autoregulation: a crucial step for cell-fate determination. Trends Immunol 27, 461-469). Consistent with this possibility, we observed that levels of mRNAs encoding NFATc1, NFATc2 and NFATc4 were higher in islets from P10 mice compared to those in P28 islets (FIG. 13A). FACS purification confirmed that NFATc1 mRNA levels were approximately 50% greater in β-cells from P5-15 islets than those in β-cells from adult mice (FIG. 13B). Thus, expression and activity of Cn/NFAT signaling appears to be enhanced in neonatal islets, when expression of NFAT targets governing β-cell proliferation and maturation peaks.

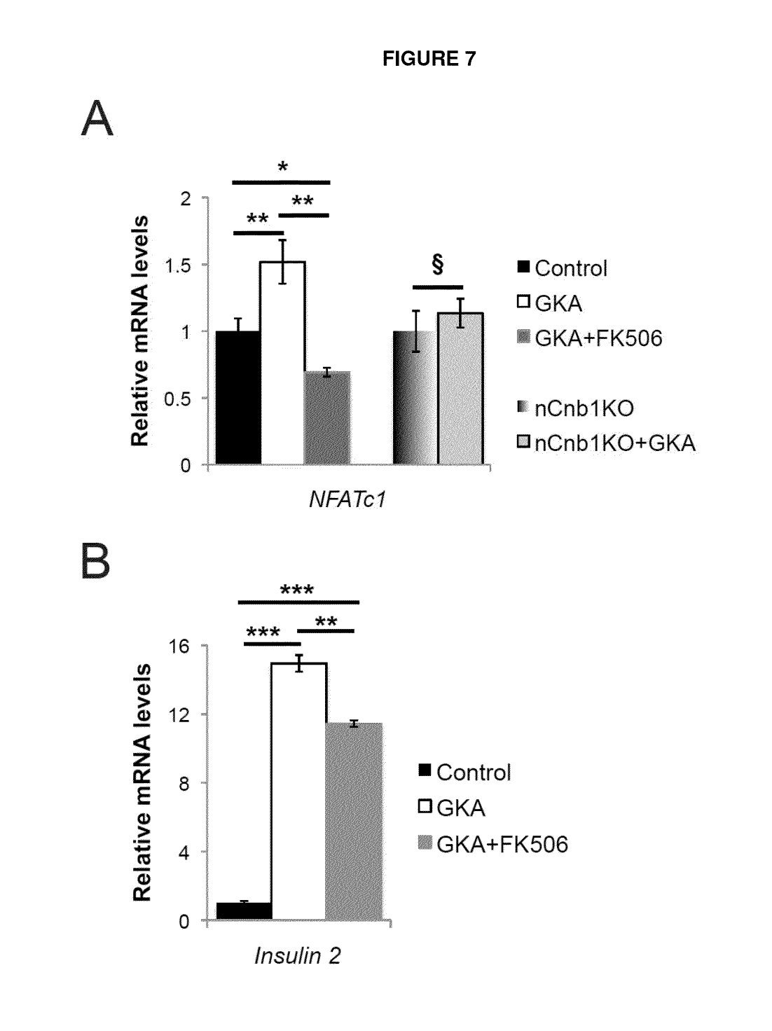

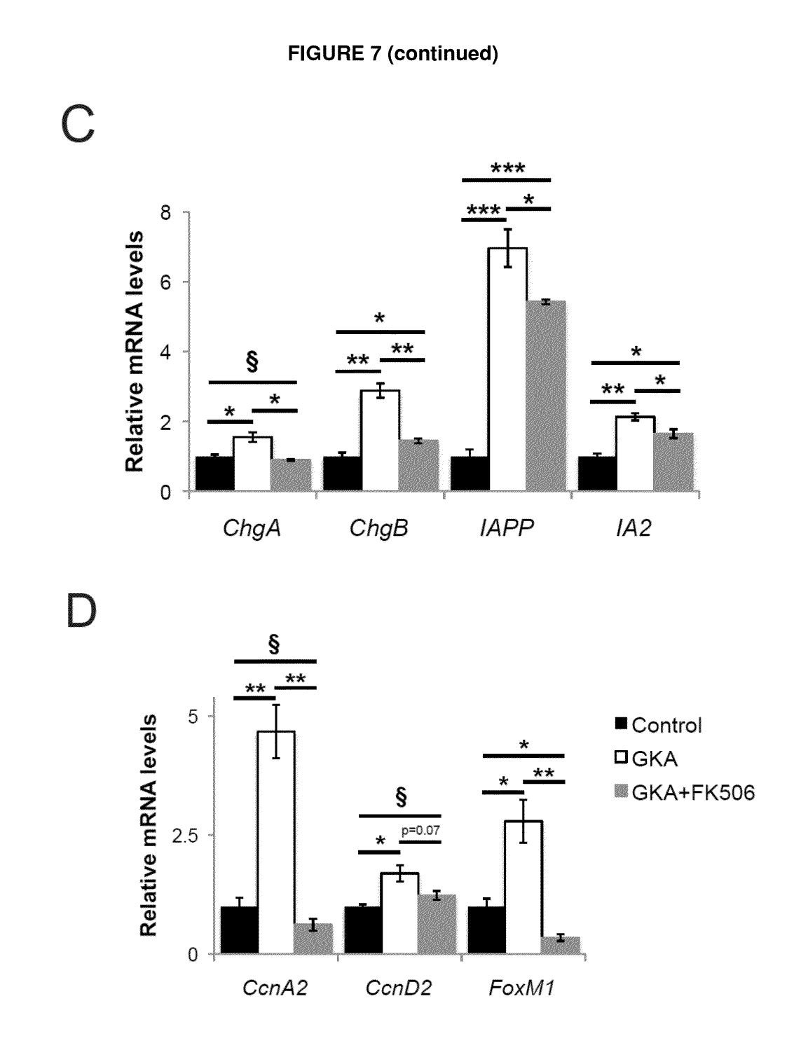

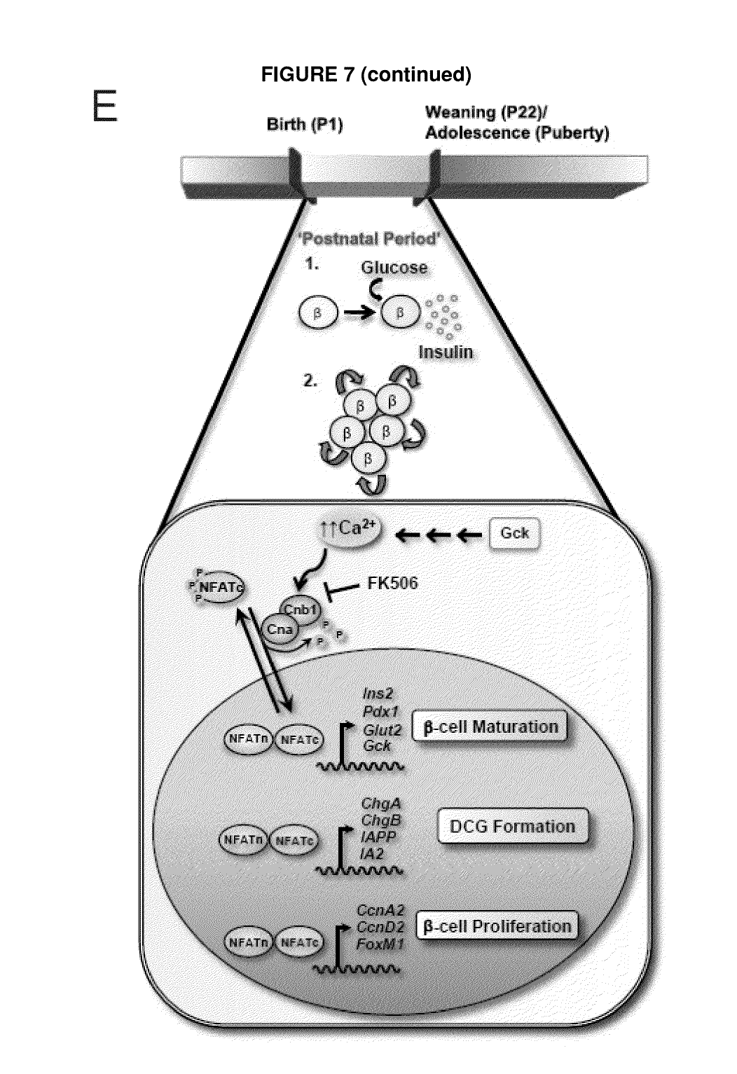

What pathways might regulate Cn/NFAT signaling during postnatal β-cell development? Prior work suggests that glucokinase activation is a physiological mechanism for stimulating depolarization and Ca²⁺-dependent β-cell proliferation and maturation (Grimsby J, et al. (2003). Allosteric activators of glucokinase: potential role in diabetes therapy. Science 301, 370-3; Porat, S., et al. (2011), supra; Salpeter, S. J., et al. (2011), supra). Thus we posultated that β-cell Cn/NFAT signaling might be induced by glucokinase activators. Consistent with this possibility, we found that the glucokinase activator (GKA) R0-28-1675 (Matschinsky, F. M. (2009). Assessing the potential of glucokinase activators in diabetes therapy. Nat Rev Drug Discov. 2009 May; 8(5):399-416) increased NFATc1 mRNA by 50% in cultured P10 islets (FIG. 7A), compared to islets exposed to vehicle control. This induction was blocked by simultaneous exposure of WT islets to FK506 and GKA (FIG. 7A), and consistently, GKA-induced expression of NFATc1 was blocked in nCnb1KO islets (FIG. 7A). WT islets exposed to GKA had significantly elevated Insulin 2 (Ins2) levels, an effect blunted by FK506 (FIG. 7B), consistent with findings here and by others (Lawrence et al. (2001), supra; Lawrence et al. (2009), supra). In addition to Insulin, mRNAs encoding other components of β-cell dense core vesicles, including ChgA, ChgB, IAPP, and IA2 were significantly increased in neonatal P10 islets exposed to GKA, an effect eliminated or significantly reduced by FK506 (FIG. 7C). Similar induction of mRNAs encoding β-cell cycle regulators CcnA2, CcnD2 and FoxM1 were observed in P10 islets exposed to R0-28-1675; again, these effects were attenuated or blocked by simultaneous exposure to FK506 (FIG. 7D). Together, these findings suggest that glucokinase activation of Cn/N FAT signaling in postnatal islets may regulate crucial regulators of β-cell function and proliferation.

### Discussion

Progress in creating replacement islets from renewable sources, like embryonic stem cells, has been limited by a lack of knowledge about physiological mechanisms promoting development of functional insulin-secreting β-cells (McKnight et al. (2010), supra). Likewise, the promise of advances in human islet transplantation has been constrained by a demand for replacement β-cells that exceeds supply (Meier et al. (2008) Beta-cell replication is the primary mechanism subserving the postnatal expansion of beta-cell mass in humans. Diabetes 57, 1584-1594). Accordingly, there is intense interest in understanding the mechanisms regulating the in vivo maturation and proliferation of pancreatic islet β-cells. Here we report that the Cn/N FAT pathway regulates both the maturation and expansion of functional β-cells in both mouse and human islets.

During physiological growth in neonatal mice and humans, islet β-cells adapt by enhancing their hallmark functions, including glucose sensing, insulin production, dense core vesicle development, and stimulus-secretion coupling (Bruin et al. (2008), supra; Kim et al. (2006), supra; Suckale and Solimena (2010), supra). This functional maturation likely reflects the shift from intrauterine energy sources to the postnatal diet (Fowden and Hill (2001) Intra-uterine programming of the endocrine pancreas. Br. Med. Bull 60, 123-142). Expression of genes encoding the effectors of these functional adaptations, including Insulin2, Glut2, Glucokinase, Pdx1, ChromograninA, and IA2, increases in neonatal development (Aguayo-Mazzucato et al. (2011), supra; Jermendy et al. (2011), supra). Prior studies (Ahlgren, U., et al. (1998) beta-cell-specific inactivation of the mouse Ipf1/Pdx1 gene results in loss of the beta-cell phenotype and maturity onset diabetes. Genes Dev 12, 1763-1768; Gu et al. (2010) Pancreatic beta cells require NeuroD to achieve and maintain functional maturity. Cell Metab 11, 298-310; Mziaut et al. (2008) ICA512 signaling enhances pancreatic beta-cell proliferation by regulating cyclins D through STATs. Proc. Natl. Acad. Sci. U.S.A 105, 674-679; Zhang et al. (2005) MafA is a key regulator of glucose-stimulated insulin secretion. Mol. Cell. Biol 25, 4969-4976) suggest that basal expression of a subset of these β-cell factors in adult islets or cell lines is governed by transcriptional regulators including MafA, Pdx1, NeuroD1, IA2 and Stat5. Studies by our group and others have previously demonstrated regulation of Insulin by NFATc1 in the β-cell line MIN6 and in adult islets (Heit et al. (2006a), supra; Lawrence et al. (2001) supra). However, the basis for dynamic changes in neonatal expression of these β-cell factors in mouse or human islets has not been established. Thus, studies here demonstrating in vivo roles for Cn/N FAT in mouse islet maturation, and for sustained expression of these hallmark β-cell developmental regulators in human islets is unprecedented. Evidence is also presented here that NFATc1 is sufficient to activate expression of target genes encoding β-cell cycle regulators and dense core vesicle components. However, our findings do not rule out roles for other calcineurin-regulated factors in neonatal β-cell development. In addition to NFATs, calcineurin has other important targets including TORC2 and Erk1/2 (Arnette et al. (2003) Regulation of ERK1 and ERK2 by glucose and peptide hormones in pancreatic beta cells. J. Biol. Chem. 278, 32517-32525; Lawrence et al. (2005) ERK1/2-dependent activation of transcription factors required for acute and chronic effects of glucose on the insulin gene promoter. J. Biol. Chem 280, 26751-26759; Le Lay et al. (2009) CRTC2 (TORC2) contributes to the transcriptional response to fasting in the liver but is not required for the maintenance of glucose homeostasis. Cell Metab. 10, 55-62). However, glucose regulation was not detectably perturbed in mice lacking TORC2 (Le Lay et al. (2009), supra) and a prior study did not observe impaired insulin secretion following Erk1/2 blockade in cultured cells (Khoo and Cobb (1997) Activation of mitogen-activating protein kinase by glucose is not required for insulin secretion. Proc Natl Acad Sci USA 94, 5599-604).

How are β-cell growth and maturation coordinated in neonatal islets? Prior reports suggested that glucose metabolism by glucokinase may link β-cell depolarization and Ca²⁺ influx to β-cell proliferation (Pechhold et al. (2009), supra; Porat et al. (2011), supra; Salpeter et al. (2011), supra), and function (Salpeter et al. (2011), supra; Terauchi et al. (1995) Pancreatic beta-cell-specific targeted disruption of glucokinase gene. Diabetes mellitus due to defective insulin secretion to glucose. J Biol Chem 270, 30253-6; Terauchi et al. (2007) Glucokinase and IRS-2 are required for compensatory beta cell hyperplasia in response to high-fat diet-induced insulin resistance. J Clin Invest 117, 246-57; Vionnet et al. (1992) Nonsense mutation in the glucokinase gene causes early-onset non-insulin-dependent diabetes mellitus. Nature 356, 721-2). Moreover, neonatal β-cell growth and maturation in rodents is accompanied by enhanced Ca²⁺ flux (Navarro-Tableros et al. (2007) Physiological development of insulin secretion, calcium channels, and GLUT2 expression of pancreatic rat beta-cells. Am. J. Physiol. Endocrinol. Metab. 292, E1018-1029), requiring the voltage-gated calcium channel subunit α1D (Namkung et al. (2001) Requirement for the L-type Ca (2+) channel alpha(1D) subunit in postnatal pancreatic beta cell generation. J. Clin. Invest. 108, 1015-1022). However, it remained unclear how Ca²⁺ signals were connected to genetic programs controlling β-cell growth and maturation. Cn/NFAT signaling is regulated by Ca²⁺ transients (reviewed in Crabtree and Olson (2002) supra), and Ca²⁺-regulation of Cn/NFAT activity in β-cells has been firmly established (Lawrence et al. (2002) NFAT regulates insulin gene promoter activity in response to synergistic pathways induced by glucose and glucagon-like peptide-1. Diabetes 51, 691-698; Lawrence et al. (2009), supra). Our findings link glucokinase activation to Cn/NFAT signaling induction in mouse islet β-cells. Thus, findings here and by others suggest that Cn/NFAT signaling is a crucial pathway that links enhanced glucose metabolism and Ca2+ dynamics to transcriptional regulation that drives β-cell proliferation and maturation in neonatal islets (FIG. 7E). We speculate that, like in development and maturation of lymphocytes, further studies may reveal if Cn/NFAT signaling converts and integrates activity dependent β-cell Ca2+ transients into gene expression changes that orchestrate developmental growth and functional maturation. Further studies are also required to establish if glucokinase activators can stimulate expression of β-cell cycle regulators and hallmark dense core vesicle components in human islets in culture and in vivo. If so, discovery of Cn/NFAT activators might be used to stimulate proliferation and expansion of functional human β-cells produced from expandable sources, including stem cell lines.

Based on the high incidence of diabetes mellitus observed in patients administered the calcineurin inhibitors FK506 or cyclosporine A (Heit et al. (2007) Calcineurin/NFAT signaling in the beta-cell: From diabetes to new therapeutics. Bioessays 29, 1011-1021; Montori et al. (2002) Posttransplantation diabetes: a systematic review of the literature. Diabetes Care 25, 583-592; Oetjen et al. (2003) Inhibition of human insulin gene transcription by the immunosuppressive drugs cyclosporin A and tacrolimus in primary, mature islets of transgenic mice. Mol. Pharmacol 63, 1289-1295), we and others postulated that disrupted Cn/NFAT signaling might impair β-cell function (Heit et al. (2006a), supra; Redmon et al. (1996) Effects of tacrolimus (FK506) on human insulin gene expression, insulin mRNA levels, and insulin secretion in HIT-T15 cells. J. Clin. Invest 98, 2786-2793). Supporting this view, cultured human islets exposed to FK506 have impaired insulin secretion (Johnson et al. (2009) Different effects of FK506, rapamycin, and mycophenolate mofetil on glucose-stimulated insulin release and apoptosis in human islets. Cell Transplant 18, 833-845) and reduced DCG numbers (Bugliani et al. (2009) The direct effects of tacrolimus and cyclosporin A on isolated human islets: A functional, survival and gene expression study. Islets 1, 106-110; Drachenberg et al. (1999) Islet cell damage associated with tacrolimus and cyclosporine: morphological features in pancreas allograft biopsies and clinical correlation. Transplantation 68, 396-402), but the molecular basis for these findings remained unclear. Our results argue that impaired islet insulin secretion from exposure to calcineurin inhibitors reflects both disrupted expression of β-cell secretion regulators like Glut2 and Glucokinase and impaired biogenesis of β-cell dense core vesicles. Our findings from genetic, pharmacological, ultrastructural and molecular studies, including ChIP, show that NFAT regulates expression of genes encoding the principal components of mouse and human β-cell DCGs, including Insulin, Chromogranins A and B, IA2 and IAPP. Our findings also complement prior in vitro studies of cultured rodent insulinoma cell lines and islets demonstrating that β-cell depolarization may induce Ca²⁺-dependent processing of IA2 by the calpain protease, leading to Stat5 activation and increased expression of IA2 itself and other DCG components (Mziaut et al. (2006), supra; Trajkovski et al. (2004) supra). Here we show that Cn/NFAT signaling regulates islet expression of IA2 and Nfatc1. Recent studies have shown that calpain may also activate calcineurin activity in response to Ca²⁺ signaling (Chang et al. (2004) Role of calcium in pancreatic islet cell death by IFN-gamma/TNF-alpha. J. Immunol 172, 7008-7014). Thus, Ca²⁺-dependent signaling in β-cells may lead both to auto-activation and cross-activation of transcriptional regulators that control expression and assembly of key DCG components, as well as essential glucose sensing factors like glucose transporters and glucokinase. Studies of cultured cell lines and rodent islets provide evidence that signaling pathways regulated by GLP1, ERK/MAPK and glucose may activate the Cn/NFAT pathway in β-cells (Lawrence et al. (2001), supra; Lawrence et al. (2005), supra; Lawrence et al. (2009), supra). Additional studies are needed to test if these or other factors that modulate β-cell Cn/NFAT activation may prove fruitful for attempts to direct the functional maturation of replacement islet cells from multipotent stem cell sources.

Prior studies suggest that during the lifetime of mice and humans, β-cell proliferation and expansion is maximal in neonates, a period of rapid host growth (Butler et al. (2007) The replication of beta cells in normal physiology, in disease and for therapy. Nat Clin Pract Endocrinol Metab 3, 758-768; Meier et al. (2008) Beta-cell replication is the primary mechanism subserving the postnatal expansion of beta-cell mass in humans. Diabetes 57, 1584-1594; Teta et al. (2005) Very slow turnover of beta-cells in aged adult mice. Diabetes 54, 2557-2567). CyclinD1, CyclinD2, Cdk4 and FoxM1 are required for β-cell proliferation in this period of physiological juvenile growth (Georgia and Bhushan (2004), supra; Kushner et al. (2005), supra; Rane et al. (1999), supra; Zhang et al. (2006), supra). Levels of CcnD2 and FoxM1 mRNA and protein peak in juvenile islets (Georgia and Bhushan (2004), supra; Zhang et al. (2006) supra) suggesting transcriptional mechanisms govern expression of these β-cell growth regulators. Work here reveals that Cn/NFAT signaling regulates in vivo β-cell expression of CcnD2 and FoxM1, as well as CcnA2, a known regulator of proliferation in smooth muscle and fibroblasts (Karpurapu et al. (2008) NFATc1 targets cyclin A in the regulation of vascular smooth muscle cell multiplication during restenosis. J. Biol. Chem 283, 26577-26590; Tomono et al. (1998) Inhibitors of calcineurin block expression of cyclins A and E induced by fibroblast growth factor in Swiss 3T3 fibroblasts. Arch. Biochem. Biophys 353, 374-378). Studies of neonatal mouse islets exposed to FK506 demonstrate Cn-dependent association of NFATc1 with these loci, supporting the view that NFATc1 regulates expression of these genes. Genetic studies allowing simultaneous inactivation of the eight alleles encoding NFATc1-c4 in β-cells should test the requirement for these transcriptional regulators in neonatal β-cell proliferation. Collectively, the results presented here indicate that Cn/NFAT activity governs the expression of multiple cell cycle regulators essential for establishing β-cell mass in neonatal mice.

In contrast to mice, virtually nothing is known about the mechanisms regulating β-cell proliferation in human juvenile islets (McKnight et al. (2010), supra). Here we show levels of mRNA encoding CCND2, FOXM1 and CCNA2 are highest in islets from young human donors, then decline in adult islets, similar to their age-dependent reduction in mice. Moreover, molecular analysis reveals that Cn/NFAT signaling is required to sustain expression of these factors, and β-cell proliferation. Additional studies should also reveal whether age-dependent decline of human β-cell proliferation is linked to attenuation of Cn/NFAT activity. The work presented here unveils evolutionarily-conserved mechanisms governing human β-cell proliferation, and suggests that impaired β-cell replication may underlie iatrogenic diabetes in patients exposed to calcineurin inhibitors, like FK506. Development of novel Cn/NFAT pathway inhibitors with improved therapeutic index (Bernard et al. (2010) Hypoglycaemia following upper gastrointestinal surgery: case report and review of the literature. BMC Gastroenterol 10, 77) might prove useful to develop strategies for diseases reflecting increased β-cell mass or function, including congenital or acquired hyperinsulinism, nesidioblastosis following bariatric surgery, insulinomas, and other neuroendocrine cancers.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of modulating β cell development, the method comprising:
contacting a progenitor cell with an effective amount of an agent that modulates calcineurin/NFAT signaling, and
assaying for the development of a mature β cell.

2. The method according to claim 1, wherein the agent promotes calcineurin/NFAT signaling, wherein β cell development is promoted.

3. The method according to claim 2, wherein the agent is selected from the group consisting of ionomycin, calcimycin, a glucokinase activator, glucagon-like peptide 1 (GLP1), and phorbol-12 myristate-13 acetate (PMA).

4. The method according to claim 1, wherein the agent antagonizes calcineurin/NFAT signaling, wherein β cell development is inhibited.

5. The method according to claim 4, wherein the agent is selected from the group consisting of tacrolimus (FK506), pimecrolimus, cyclosporine A, 11R-VIVIT, INCA-1, INCA-2, and INCA-6.

6. The method according to claim 1, wherein the progenitor cell is an embryonic stem (ES) cell, an induced pluripotent stem cell (iPSC), an endocrine progenitor cell (EPC), or a pancreatic duct cell.

7. The method according to claim 1, wherein the method occurs in vitro.

8. The method according to claim 1, wherein the method occurs in vivo.

9. A method of producing an enriched composition of mature β cells in vitro, the method comprising:
contacting a progenitor cell with an effective amount of an agent that promotes calcineurin/NFAT signaling under conditions that promote β cell development in vitro,
wherein an enriched population of mature β cells is produced.

10. The method according to claim 9, wherein the agent that promotes calcineurin/N FAT signaling comprise glucokinase activator (GKA).

11. The method according to claim 9, wherein the method further comprises contacting the mature β cell with an agent that promotes mature β cell expansion.

12. The method according to claim 9, wherein the method further comprises enriching the composition for mature β cells by affinity separation.

13. The method according to claim 9, wherein the method further comprises assaying the population for mature β cells.

14. An enriched composition of β cells prepared by the method of claim 9.

15. A method of treating an individual in need of functional β cells, comprising:
transplanting an enriched composition of β cells prepared by the method of claim 9 into the individual.

16. The method according to claim 15, wherein the progenitor cell is from the individual.

17. The method according to claim 16, wherein the individual has diabetes.

18. A method of suppressing the development of mature β cells in an individual, comprising:
contacting pancreatic tissue in vivo with an effective amount of an agent that inhibits calcineurin/NFAT signaling, wherein β cell development is suppressed

19. The method according to claim 18, wherein the agent is selected from the group consisting of tacrolimus (FK506), pimecrolimus, cyclosporine A, 11R-VIVIT, INCA-1, INCA-2, and INCA-6.

20. The method according to claim 22, wherein the individual has insulinoma, hypoglycemia, or an acquired state of β cell overgrowth.


SEQUENCE LISTING (42 sequences)

SEQ ID NO: 1 - DNA, 20 bp, Artificial Sequence
  Synthetic oligonucleotide
gcctggcactcaagagatcc

SEQ ID NO: 2 - DNA, 20 bp, Artificial Sequence
  Synthetic oligonucleotide
tgaagttccactgacccaaa

SEQ ID NO: 3 - DNA, 20 bp, Artificial Sequence
  Synthetic oligonucleotide
agagggcctcggagaagtag

SEQ ID NO: 4 - DNA, 20 bp, Artificial Sequence
  Synthetic oligonucleotide
caagctggaagggcagttag

SEQ ID NO: 5 - DNA, 20 bp, Artificial Sequence
  Synthetic oligonucleotide
tcaaagcagctctcccttct

SEQ ID NO: 6 - DNA, 20 bp, Artificial Sequence
  Synthetic oligonucleotide
cgcagcctcctgtgataact

SEQ ID NO: 7 - DNA, 20 bp, Artificial Sequence
  Synthetic oligonucleotide
agtttcagctgtgccacctt

SEQ ID NO: 8 - DNA, 20 bp, Artificial Sequence
  Synthetic oligonucleotide
caatgctatgccggctttta

SEQ ID NO: 9 - DNA, 20 bp, Artificial Sequence
  Synthetic oligonucleotide
gagaaagagggggagaggaa

SEQ ID NO: 10 - DNA, 20 bp, Artificial Sequence
  Synthetic oligonucleotide
aaatcaaacaggccaaagga

SEQ ID NO: 11 - DNA, 20 bp, Artificial Sequence
  Synthetic oligonucleotide
tccaagacatccagggctac

SEQ ID NO: 12 - DNA, 20 bp, Artificial Sequence
  Synthetic oligonucleotide
tgacatttggggtgtgtttg

SEQ ID NO: 13 - DNA, 20 bp, Artificial Sequence
  Synthetic oligonucleotide
aactggttcatcaggccatc

SEQ ID NO: 14 - DNA, 20 bp, Artificial Sequence
  Synthetic oligonucleotide
actgggtccccactaccttt

SEQ ID NO: 15 - DNA, 20 bp, Artificial Sequence
  Synthetic oligonucleotide
gaaggagaaggggaaggaga

SEQ ID NO: 16 - DNA, 20 bp, Artificial Sequence
  Synthetic oligonucleotide
atgttcagggcttgttcagg

SEQ ID NO: 17 - DNA, 20 bp, Artificial Sequence
  Synthetic oligonucleotide
aatttttggcaagtggctgt

SEQ ID NO: 18 - DNA, 22 bp, Artificial Sequence
  Synthetic oligonucleotide
tttgaagcctataaagcggtct

SEQ ID NO: 19 - DNA, 20 bp, Artificial Sequence
  Synthetic oligonucleotide
ttggcgtgctacacctacag

SEQ ID NO: 20 - DNA, 20 bp, Artificial Sequence
  Synthetic oligonucleotide
cccctcctcctttcaatctc

SEQ ID NO: 21 - DNA, 20 bp, Artificial Sequence
  Synthetic oligonucleotide
aggggcaaaagacaggtttc

SEQ ID NO: 22 - DNA, 21 bp, Artificial Sequence
  Synthetic oligonucleotide
tcaaagctcggctttagttga

SEQ ID NO: 23 - DNA, 20 bp, Artificial Sequence
  Synthetic oligonucleotide
gtcaggtggcaaagagcttc

SEQ ID NO: 24 - DNA, 20 bp, Artificial Sequence
  Synthetic oligonucleotide
ccttgcaacacctacccatt

SEQ ID NO: 25 - DNA, 22 bp, Artificial Sequence
  Synthetic oligonucleotide
tgactgaaagaggaattgagga

SEQ ID NO: 26 - DNA, 20 bp, Artificial Sequence
  Synthetic oligonucleotide
aagtgcagccggagaatatg

SEQ ID NO: 27 - DNA, 20 bp, Artificial Sequence
  Synthetic oligonucleotide
ggcggttttgcagtcatatt

SEQ ID NO: 28 - DNA, 20 bp, Artificial Sequence
  Synthetic oligonucleotide
ctaaaacagggccaatggaa

SEQ ID NO: 29 - DNA, 24 bp, Artificial Sequence
  Synthetic oligonucleotide
tcattatgcatttctgtccttttt

SEQ ID NO: 30 - DNA, 20 bp, Artificial Sequence
  Synthetic oligonucleotide
gctctttcaccacgaccact

SEQ ID NO: 31 - DNA, 20 bp, Artificial Sequence
  Synthetic oligonucleotide
ggagctattcagcgtgcttc

SEQ ID NO: 32 - DNA, 20 bp, Artificial Sequence
  Synthetic oligonucleotide
ttcgtgagtctgcccttctt

SEQ ID NO: 33 - DNA, 20 bp, Artificial Sequence
  Synthetic oligonucleotide
tcaagcatgcgttagagcac

SEQ ID NO: 34 - DNA, 20 bp, Artificial Sequence
  Synthetic oligonucleotide
ggcgagtgagggattaggtc

SEQ ID NO: 35 - DNA, 20 bp, Artificial Sequence
  Synthetic oligonucleotide
tcgtgacctcaagtgatcca

SEQ ID NO: 36 - DNA, 20 bp, Artificial Sequence
  Synthetic oligonucleotide
cgctaggccctgaagataca

SEQ ID NO: 37 - DNA, 20 bp, Artificial Sequence
  Synthetic oligonucleotide
tctgcccaaactctgtaccc

SEQ ID NO: 38 - DNA, 20 bp, Artificial Sequence
  Synthetic oligonucleotide
cttgaacccaagaggtggag

SEQ ID NO: 39 - DNA, 20 bp, Artificial Sequence
  Synthetic oligonucleotide
gattacaggcgtgagcttcc

SEQ ID NO: 40 - DNA, 20 bp, Artificial Sequence
  Synthetic oligonucleotide
aagaccacagccacagaaca

SEQ ID NO: 41 - DNA, 20 bp, Artificial Sequence
  Synthetic oligonucleotide
ggaggggagagaggatatgg

SEQ ID NO: 42 - DNA, 20 bp, Artificial Sequence
  Synthetic oligonucleotide
tctcgatctcctgacctcgt